(19) United States
(12) Patent Application Publication
(10) Pub. No.: US 2004/0092446 A1
(43) Pub. Date: May 13, 2004

(54) HA-1 epitopes and uses thereof

(76) Inventors: Elsa A.J.M. Goulmy, Oegstgeest (NL); Donald F. Hunt, Charlottesville, VA (US); Victor H. Engelhard, Charlottesville, VA (US)

Correspondence Address: TRASK BRITT, P.O. BOX 2550, SALT LAKE CITY, UT, 84110, US

(21) Appl. No.: 10/623,176

(22) Filed: Jul. 18, 2003

Related U.S. Application Data
(63) Continuation-in-part of application No. 09/489,760, filed on Jan. 21, 2000.

(30) Foreign Application Priority Data
Jul. 23, 1997 (EP) 97202303.0
Jul. 23, 1998 (WO) PCT/NL98/00424

(51) Int. Cl. A61K 38/17; C07K 14/74

(52) U.S. Cl. 514/12; 530/350

(57) ABSTRACT

Peptide sequences constituting T-cell epitopes of minor Histocompatibility antigen, HA-1. HA-1 is associated with Graft versus Host Disease. The peptides and their derivatives find many uses, for instance in bone marrow transplantation, organ transplantation and in treatment of leukemia and non-hematopoietic tumors. The peptide and/or its derivatives can be incorporated in vaccines, in pharmaceutical formulations and they can be used in diagnostic test kits. HA-1 is expressed by non-hematopoietic tumor cells. While absent in normal epithelial cells, tumor cells and tumor cell lines, particularly from epithelial origin, express HA-1 and are recognized by HA-1 cytotoxic T cells. The invention provides means and methods for HA-1 specific immunotherapy for HA-1 positive patients with non-hematopoietic tumor cells.

### CROSS-REFERENCE TO RELATED APPLICATION

This application is a continuation in part of co-pending application U.S. Ser. No. 09/489,760, filed on Jan. 21, 2000, the contents of the entirety of which is incorporated by this reference.

### TECHNICAL FIELD

The invention relates generally to biotechnology, and more particularly to the field of cellular immunology.

### BACKGROUND

Bone marrow transplantation (BMT), one of the areas the invention is concerned with and the area from which the present invention originates, finds its application in the treatment of for instance severe aplastic anemia, leukemia and immune deficiency diseases.

In the early days of this technique many transplants failed through rejection of the graft by the host. Transplants that did succeed, however often led to an immune response by lymphocytes present in the graft against various tissues of the host (GvHD). It is now known that the GvHD response is mainly due to the presence of major histocompatibility (H) antigens which present a transplantation barrier. Therefore, it is now routine practice to graft only HLA-matched materials (either from siblings or unrelated individuals) resulting in a much improved rate of success in bone marrow transplantation. However, despite this improvement, as well as improvements in pre-transplantation chemotherapy or radiotherapy and the availability of potent immuno-suppressive drugs, about 20-70% of the treated patients still suffer from GvHD (the percentage is age and bone marrow donor dependent). To avoid GvHD it has been suggested to remove the cells (mature T cells) causing the reaction from the graft. This however often leads to graft failure or to recurrence of the original disease. The cells responsible for GvHD are also the cells which often react against the original aberrant cells in for instance leukemia (Graft versus Leukemia response).

Since BMT is nowadays mainly carried out with HLA matched grafts, the GvHD which still occurs must be caused by another group of antigens. It is very likely that the group of so called minor H antigens (mHag), which are non-MHC encoded histocompatibility antigens (unlike the major H antigens) are at least partially responsible for the remaining incidence of GvHD. mHag's have originally been discovered in congeneic strains of mice in tumor rejection and skin rejection studies. In mice, the use of inbred strains has shown that mHag are encoded by almost 50 different allelically polymorphic loci scattered throughout the genome. In humans, although cumbersome to identify, mHag have been shown to exist, but their overall number and complexity remains uncertain. Minor H antigens are most likely quite different from each other and quite different from major H antigens, they are probably a diverse and elusive group of fragments of molecules which are participating in various cellular housekeeping functions. Their antigenicity may come very incidentally, as naturally processed fragments of polymorphic proteins that associate with MHC products. Some of the mH antigens appear to be widely expressed on various tissues throughout the body whereas others show limited tissue distribution.

One of the better known minor histocompatibility antigens is the H-Y antigen. H-Y is an mH antigen that can lead to rejection of HLA-matched male organ and bone marrow grafts by female recipients, and to a higher incidence of GvHD in female-to-male grafts, particularly if the female donor had been previously pregnant. The H-Y antigen may also play a role in spermatogenesis. The human H-Y antigen is an 11 residue peptide derived from SMCY, an evolutionary conserved Y chromosomal protein. Another well known mH antigen that can lead to GvHD is the HA-2 antigen. The human HA-2 antigen is an 9 residue peptide likely derived from a class I myosin. However, the nature of the HA-1 antigen, responsible for a majority of current cases of GvHD has remained elusive so far. Human bone marrow transplants performed as therapeutic treatment of severe aplastic anemia, leukemia and immune deficiency disease became available in the seventies. For the present, the long-term results of allogeneic bone marrow transplantation (BMT) have greatly improved due to the use of HLA-matched siblings as marrow donors, advanced pre-transplant chemoradiotherapy, the use of potent immunosuppressive drugs as GVHD prophylaxis, better antibiotics and isolation procedures. Nonetheless, the results of clinical BMT reveal that the selection of MHC identical donors/recipients is not a guarantee of avoidance of GVHD or disease free survival even when donor and recipient are closely related. Allogeneic BMT especially in adults results, depending on the amount of T cell depletion of the graft, in up to 80% of the cases in GVHD. In the HLA genotypically identical situation it amounts to 15-35% whereas in the phenotypical HLA matched recipient/donor combinations, the occurrence of GVHD is significantly higher i.e., 50-80%. Disparities for minor Histocompatibility antigens (mHag) between donor and recipient constitute a potential risk for GVHD or graft failure, which necessitate life long pharmacologic immunosuppression of organ and bone marrow transplant recipients. It is also believed that mHag are involved in the “beneficial” side effect of GVHD i.e., the Graft-versus-Leukemia activity. Several reports demonstrated the presence of anti-host mHag specific CTL in patients suffering from GVHD after HLA genotypically identical BMT. In our laboratory, much effort was put into the further characterization of a number of anti-host mHag specific CTLS. Hereto, CTL clones specific for host mHag were isolated from the peripheral blood (PBL) of patients suffering from severe GvHD. mHag HA-1 specific CD8⁺ CTL clones were originally obtained after restimulation of in vivo primed PBLs from three patients suffering from GvHD after HLA identical but mHag nonidentical BMT. The post BMT CTL lines were cloned by limiting dilution, resulting in the isolation of a large number of mHag-specific CTL clones. Subsequent immunogenetic analyses revealed that the CTL clones (as described above) identified five non-sex-linked mHag, designated HA-1, -2, -3, -4, -5, which are recognized in a classical MHC restricted fashion. mHag HA-3 was recognized in the presence of HLA-A1 and mHag HA-1, -2, -4 and 5 were found to require the presence of HLA-A2. Segregation studies demonstrated that each of mHag HA-1 to HA-5 is the product of a single gene segregating in a Mendelian fashion and that HA-1 and HA-2 are not coded within the HLA region. The mHag differ from each other in phenotype frequencies: mHag HA-1 appeared relatively frequent (i.e., 69%) whereas mHag HA-2 appeared very frequent (i.e., 95%) in the HLA-A2 positive healthy population. An inventory in five patients of mHag HA-1, -2, -3, -4 and -5 specific anti-host CTL responses after BMT demonstrated in 3 patients clones specific for the mHag HA-1. This observation points towards the immunodominant behavior of mHag HA-1.

With regard to the mHag expressed on different tissues, we observed ubiquitous versus restricted tissue distribution of the mHag analyzed. The expression of the mHag HA-1 was observed to be restricted to the cells of the hematopoietic cell lineage, such as thymocytes, peripheral blood lymphocytes, B cells, monocytes. Also the bone marrow derived professional APCs: the dendritic cells and the epidermal Langerhans cells were found to express the mHag HA-1. The mHag HA-1 was also found to be expressed on clonogenic leukemic precursor cells as well as on freshly isolated myeloid and lymphoid leukemic cells, indicating that mHag specific CTLs are capable of HLA class I restricted antigen specific lysis of leukemic cells. To substantiate the importance of the human mH antigenic systems, we investigated whether the mHag are conserved in evolution between human and non human primates. Hereto, cells from non human primates were transfected with the human HLAA2.1 gene. Subsequent analyses with our human allo HLA-A2.1 and four mHag A2.1 restricted CTL clones revealed the presentation of ape and monkey allo and mHag HY, HA-1 and HA-2 peptides in the context of the transfected human HLAA2.1 molecule by ape and monkey target cells. This implicates that the HA-1 peptide is conserved for at least 35 million years. A prospective study was performed in order to document the effect and clinical relevance of mHag in HLA genotypically identical BMT on the occurrence of acute (grade ≧2) GVHD. The results of the mHag typing using the CTL clones specific for five well defined mHag HA-1 to HA-5 demonstrated a significant correlation between mHag HA-1, -2, -4 and -5 mismatch and GVHD. A significant correlation (P=0.024) with the development of GVHD was observed when analyzed for only mHag HA-1. To analyze a putative peptidic nature of the mHag HA-1, we analyzed the requirement of the MHC encoded TAP1 and TAP2 gene products for mHag peptide presentation on the cell surface. The transporter genes TAP1 and TAP2 associated with antigen presentation are required for delivery of peptides from the cytosol with the endoplasmic reticulum. The availability of a human cell line “T2” lacking both transport and proteasome subunit genes enabled us to study the processing and presentation of human mHag. We demonstrated that the (rat) transport gene products TAP1 and TAP2u were required for processing and presentation of antigenic peptides from the intracellular mH protein HA-1. Information on the TCR repertoire post BMT in man is extremely scarce. We have analyzed the composition of the T cell receptor (TCR) V region of mHag HA-1 specific CD8+ CTL clones by DNA sequencing of the α and β chains. We observed by analyzing TCR usage of 12 clones derived from 3 unrelated individuals that the TcRβ chains all used the TCRβV6S9 gene segment and showed remarkable similarities within the N-D-N regions.

### DISCLOSURE OF THE INVENTION

However, until the present invention no one has succeeded in identifying amino acid sequences of antigenic peptides relevant to the mHag HA-1 antigen, nor has anyone succeeded in the identification of the proteins from which this antigen is derived. We have now for the first time identified a peptide which is a relevant part of mHag HA-1.

Thus this invention provides a (poly)peptide comprising a T-cell epitope obtainable from the minor Histocompatibility antigen HA-1 comprising the sequence VLXDDLLEA (SEQ ID NO.______) or a derivative thereof having similar functional or immunological properties, wherein X represents a histidine (H) or an arginine (R) residue.

The way these sequences are obtained is described in the experimental part. An important part of this novel method of arriving at the sequences is the purification and the choice of the starting material. The method is therefore also part of the scope of this invention. However, now that the sequence is known, it is of course no longer necessary to follow that method, because the peptides can easily be made synthetically, as is well known in the art. Since routine techniques are available for producing synthetic peptides, it is also within the skill of the art to arrive at analogs or derivatives of the explicitly described peptides, which analogs and/or derivatives may have the same or at least similar functional or immunological properties and or activity. On the other hand analogs which counteract the activity of the explicitly described peptides are also within the skill of the art, given the teaching of the present invention. Therefore derivatives and/or analogs, be they of the same or different length, be they agonist or antagonist, be they peptide-like or peptidomimetic, are part of the scope of this invention.

The invention provides a (poly) peptide which can be functionally presented to the immune system in the context of the HLA-A2.1 molecule. In general peptides presented in such a context vary in length from about 7 to about 15 amino acid residues, and a polypeptide can be enzymatically processed to a peptide of such length. A peptide provided by the invention typically is at least 7 amino acids in length but preferably at least 8 or 9 amino acids. The upper length of a peptide provided by the invention is no more than about 15 amino acids, but preferably no more than about 13, preferably 11 amino acids in length. A peptide provided by the invention contains the necessary anchoring residues for presentation in the groove of the HLA-A2.1 molecule. An immunogenic polypeptide provided by the invention comprises a 7-15 amino acid long peptide, provided by the invention, optionally flanked by appropriate enzymatic cleavage sites allowing processing of the polypeptide.

A preferred embodiment of the invention is the peptide with the sequence VLHDDLLEA (SEQ ID NO.______) that induces lysis of the cell presenting it at a very low concentration of peptide present. This does not imply that peptides inducing lysis at higher concentrations are not suitable. This will for a large part depend on the application and on other properties of the peptides, which were not all testable within the scope of the present invention.

The peptides and other molecules according to the invention are usefule to induce tolerance of the donor immune system in HA-1 negative donors, so that residual peripheral blood lymphocytes in the eventually transplanted organ or the bone marrow, as it may be do not respond to host HA-1 material in an HA-1 positive recipient. In this way, GvHD will be prevented or mitigated. On the other hand, tolerance is induced in HA-1 negative recipients in basically the same way, so that upon receipt of an organ or bone marrow from an HA-1 positive donor no rejection on the basis of the HA-1 material occurs. For tolerance induction, very small doses can be given repeatedly, for instance intravenously, but other routes of administration may very well be suitable too. Another possibility is the repeated oral administration of high doses of the peptides. The peptides may be given alone, or in combination with other peptides, or as part of larger molecules, or coupled to carrier materials in any suitable excipients. Further applications of the peptide or derivatives thereof lie in the prophylactic administration of such to transplanted individuals to prevent GvHD. This can be done with either agonists, possibly in combination with an adjuvant, or with antagonists which block the responsible cells. This can be done with or without the concomitant administration of TCR derived peptide sequences or of cytokines. Furthermore, the peptides according to the invention are used to prepare therapeutic agents capable of eliminating a subset of cells, directly or indirectly, especially cells of hematopoietic origin and/or tumor cells. This can be illustrated by the following examples, which refer to leukemia related therapeutic agents.

An HA-1 positive recipient (in bone marrow transplantation) can be subjected to an additional pre-bone marrow transplant conditioning regime. This means that an agent which specifically recognizes a peptide according to the invention (an HA-1 peptide) as presented selectively on hematopoietic cells and/or tumor cells, which agent induces elimination of the cells presenting the peptide, is administered to the recipient before transplantation. This agent eliminates essentially all residual cells (leukemic cells) of hematopoietic origin as well as non-hematopoietic tumor cells, if present. Such agents include but are not limited to T cells (which are tailor made ex vivo by pulsing with the peptides provided by the invention, and optionally provided with a suicide gene) and/or antibodies coupled to toxic moieties.

An HA-1 negative donor for bone marrow transplantation can be vaccinated with a peptide according to the invention, an HA-1 peptide. Upon transplantation to an HA-1 positive recipient, the donor's immune system eliminates residual or recurrent HA-1 peptide presenting cells in the recipient which are of course leukemic or non-hematopoietic tumor cells. This is another example of tailor-made adoptive immunotherapy provided by the invention.

A transplanted HA-1 positive recipient, transplanted with HA-1 negative (or for that matter HA-1 positive) bone marrow and suffering from recurrent disease (relapse), i.e., HA-1 positive leukemic cells, is treated with (again) an agent (as above) which specifically recognizes a peptide according to the invention (an HA-1 peptide) as presented on hematopoietic cells, which agent induces elimination of the cells presenting the peptide. In case of HA-1 positive bone marrow being transplanted to the HA-1 positive recipient, it is still very important (in case of recurrent disease) to eliminate all HA-1 positive cells even though this includes the transplanted material, because otherwise the HA-1 positive leukemia will kill the recipient. To avoid the latter case, the patient can be re-transplanted if necessary.

In such therapy, protocols one may first employ adoptive immunotherapy with agents (cells, antibodies, etc.) which specifically recognize and eliminate specific peptide expressing cells (e.g., leukemic cells) that need to be destroyed, after which in a second phase the patient is reconstituted with BMT cells replacing the killed cells. The invention thus provides additional (or even substituting) protocols to other therapeutic measures such as radiation.

Other therapeutic applications of the peptide include the induction of tolerance to HA-1 proteins in HA-1 related (auto)immune diseases. On the other hand they are used in vaccines in HA-1 related (auto)immune diseases.

Diagnostic applications are clearly within the skill of the art. They include, but are not limited to HA-1 typing, detection of genetic aberrances and the like. Specific gene sequences can be detected with various methods known in the art, such as hybridization or amplification with PCR and the like. Immunological detection of peptides has also widely been practiced.

On the basis of the peptide described herein genetic probes or primers are produced which can be used to screen for the gene encoding the protein. On the other hand such probes are useful in detection kits as well. On the basis of the peptide described herein, anti-idiotypic B cells and/or T cells and antibodies are produced. Various techniques, to allow detection of suitable donors or recipients, may be used, based on amplification of HA-1 related nucleic acid sequences or on the immunological detection of HA-1 related peptide sequences. Suitable amplification or detection techniques are known in the art, and the invention enables the production of diagnostic test kits for HA-1 allelic detection and typing. The GvHD associated mH antigen HA-1 is a peptide derived from one protein allele of a di-allelic genetic system. The identification of this mH antigen HA-1 enables prospective HA-1 typing of BMT donors and recipients to improve donor selection and thereby prevention of GVHD induction. All of these embodiments have been made possible by the present disclosure and therefore are part of the present invention. The techniques to produce these embodiments are all within the skill of the art.

Furthermore, the identification of the HA-1 antigen allows the production of synthetic HA-1 peptides and peptides functionally and/or immunologically related thereto. Such peptides (which can include left or right turning residues) are designed and/or generated by various methods known in the art such as peptide synthesis and replacement mapping, followed by functional binding studies. Altered peptide ligands (APL) for the HLA-A2.1 restricted HA-1 epitope enable modification of the HA-1 directed T cell responses and thus modulate and/or mitigate the GvHD associated T cell response. In general, T cells are activated by the interaction of the T cell receptor (TCR) with the antigenic peptide in the context of a MHC molecule and can react with a number of different effector functions. APL can interact with the TCR and change the effector functions of the T cell qualitatively and/or quantitatively. APL, used in vitro as well as ex vivo can act as antagonist or agonist for the TCR and can anergize the T cells specific for the wild type peptide. An HA-1 peptide is used to induce tolerance in the living bone marrow or organ (kidney, liver, gut, skin, etc.) of HA-1 negative donors for HA-1 positive patients. In bone marrow transplantation, the peptide (given alone or in combination with others) is used to induce tolerance in the living bone marrow donor. The peptide(s) may be given orally, intravenously, intra-occularly, intranasally or otherwise. In all forms of organ, tissue and bone marrow transplantation, the HA-1 peptide is used to induce tolerance in HA-1 negative recipients.

The invention also provides an analog of the peptide according to the invention that is an antagonist for the activity of T cells recognizing the peptide. After being apprised of the disclosure given herein, such an analog may be obtained using methods and tests known in the art. Furthermore, the invention provides a method for the generation of antibodies, T cell receptors, anti-idiotypic B-cells or T-cells, comprising the step of immunization of a mammal with a peptide or a polypeptide according to the invention, and the antibodies, T-cell receptors, B-cells or T-cells obtainable by the method.

Dose ranges of peptides and antibodies and/or other molecules according to the invention to be used in the therapeutic applications as described herein before are designed on the basis of rising dose studies in the clinic in clinical trials for which rigorous protocol requirements exist.

An important advantage of using mHag-specific CTLs in adoptive immunotherapy of for example leukemia lies in their restricted and specific target cell damage. We take advantage of three of the known characteristics of human mHag i.e., 1) MHC-restricted recognition by T cells; 2) variable phenotype frequencies, i.e., mHag polymorphism; and 3) restricted tissue distribution, allowing specific and distinct targeting of mHag HA-1 related therapy. Restrictive HA-1 tissue expression significantly increases the success of adoptive immunotherapy towards various types of cancer, such as small cell lung carcinoma cells which express also the HA-1 antigen. Moreover, since mHag are clearly expressed on circulating leukemic calls and clonogenic leukemic precursor cells of both myeloid and lymphoid origin, both types of leukemias can be targeted. mHag peptide CTLs can be generated ex vivo from mHag-negative BM donors for mHag-positive patients. Peptide-specific CTL clones from an HLA-A1-positive mHag-negative healthy blood donor are generated by pulsing autologous APCs with mHag HA-1 related synthetic peptide. Proliferating clones are expanded and tested for specific cytotoxic activity. Upon transfusion (either pre-BMT as part of the conditioning regimen or post-BMT as adjuvant therapy), the mHag peptide-specific CTLs will eliminate the mHag-positive patient's leukemic cells and, if of the patient's origin, also the patient's hematopoietic cells but will spare the patient's non-hematopoietic cells. If necessary, subsequent mHag-negative donor BMT will restore the patient's hematopoietic system. A universal approach is to generate “prefab” mHag peptide-specific CTLs by using mHag-negative healthy blood donors with frequent HLA-homozygous haplotypes. Patients who are mHag-positive (and their BM donors mHag-negative) and who match the HLA typing of the CTL donor can be treated with these “ready to be used” allo-peptide specific CTLS. Transduction of these CTLs with a suicide gene allows elimination of the CTLs in case adverse effects occur.

The invention provides means and methods for eliminating a tumor cell. According to the invention, HA-1 is also present in tumor cells not of hematopoietic origin. HA-1 RNA transcription was demonstrated by the present inventor in cell lines that were generated from a wide array of tumors i.e., breast, melanomas, lung, renal cell and colon carcinomas, hepatomas and head and neck cancers. Of these cell lines, the cell lines expressing HLA-A2 and HA-1 phenotypes, were lysed by HLA-A2 restricted HA-1 specific CTL. This lysis demonstrates that HA-1 is indeed expressed in a functional way in the tested cells.

According to the invention, HA-1 is not only expressed by tumor cell lines in vitro, which are prone to mutations, but also by tumor cells in vivo. The inventor verified by RNA analysis that also primary cancer cells express HA-1. Also disseminated cancer cells express HA-1. Disseminated cells from six of fifteen patients were found to be positive for HA-1. Expression of HA-1 is not limited to a certain type of tumor cell. HA-1 expression was found on different types of carcinoma cells in the patient population.

The observation that HA-1 is expressed in a functional way on the membrane of tumor cells of non-hematopoietic origin opens the road to many different applications. One application is the use of HA-1 expression on the tumor cell to target therapy to that cell. A binding moiety capable of specifically recognizing HA-1 is used to bind and eliminate the tumor cell. In one aspect the invention therefore provides a method for eliminating a tumor cell presenting an HA-1 minor Histocompatibility antigen in the context of HLA class I, wherein the elimination is induced directly or indirectly by specific recognition of the mHag in the context, the method characterized in that the aberrant cell comprises a non-hematopoietic tumor cell that expresses HA-1. Preferably, the tumor is an epithelial tumor cell.

There are several ways to induce elimination of a cell through specific recognition of a target on that cell. In the present invention emphasis is put on specific recognition by T-cells, however, the invention is not limited to T-cells. Targeting is also possible with other binding molecules. By a binding molecule is meant herein any molecule or compound (such as for instance a cell or at least part of an antibody) capable of binding an HA-1 epitope. The HA-1 epitope may be presented in the context of MHC, but this is not necessary. The HA-1 epitope may for instance alternatively be present in the context of the normal protein. Any type of binding molecule capable of specifically recognizing the mHag in the context is suitable, provided that the molecule can mediate elimination of the cell, either directly (by means of a toxic effect) or indirectly, for instance through binding of another compound that comprises a toxic effect. Such other toxic compound for instance comprises a cytostaticum. In one embodiment, the elimination is achieved through specific recognition by a murine or human(ized) antibody specific for HA-1 or specific for HA-1 presented in the context of MHC. Humanized or human monoclonal antibodies (though with different specificities) are used in, or developed for, a great variety of anti-tumor therapies in the clinic.

A preferred means for inducing elimination of HA-1 expressing tumor cells comprises elimination induced by a T cell comprising a T cell receptor specific for HA-1 presented in the context of MHC class-I. This technology ties in with strategies for adoptive immunotherapy for hematopoietic malignancies (56). Malignant cells derived from the hematopoietic system can express HA-1 and therefore form a target for T-cells comprising a specificity for HA-1 presented in the context of MHC class-I. With the teaching of the invention it is possible to extend these approaches to any type of tumor cell of non-hematopoietic origin. The adoptive immunotherapy methods for hematopoietic malignancies are therefore also part of the invention and are incorporated herein by reference (56). The cell directly involved in killing in these adoptive immunotherapy approaches is a cytotoxic T-cell. The invention thus also provides a method for killing a human cell functionally expressing an HA-1 mHag in the context of HLA class I, comprising incubating the cell with a cytotoxic T lymphocyte (CTL) specific for the mHag presented in the context or incubating the cell with a functional equivalent of the CTL, the method characterized in that the human cell comprises a non-hematopoietic tumor cell. A CTL specific for HA-1 in the context of HLA class I is also used for determining whether a cell expresses functional levels of HA-1 in the context of HLA Class I. For instance, (tumor) cells obtained from an individual are screened for HA-1 expression to determine whether the individual is HA-1 positive. The invention therefore further provides a method for determining whether a cell expresses functional levels of an HA-1 mHag in the context of HLA class I, comprising incubating the cell with a cytotoxic T lymphocyte (CTL) specific for the HA-1 mHag presented in the context and determining whether the cell and/or the CTL is affected. There are several ways to determine whether the cell or the CTL is specifically affected by the incubation. One typically uses target cell killing to determine specific recognition by CTL, however, detection of gene expression characteristic for CTL mediated lysis in the CTL or target cell can also for instance be used.

Now that we have demonstrated that HA-1 is expressed in non-hematopoietic tumor cells, the cells can be detected and discriminated from normal cells using methods for specifically detecting HA-1 in a cell. Typically, though not necessarily, the detection methods utilize binding molecules capable of binding specifically to HA-1 and/or nucleic acid encoding the HA-1. For detection it is not required that HA-1 is presented in the context of MHC-1. Indeed preferably, the HA-1 or HA-1 encoding nucleic acid is present in the context of the normal protein/gene. The invention therefore further provides a method for marking a non-hematopoietic tumor cell comprising incubating the cell with a molecule capable of specifically binding to an HA-1 mHag presented in the context of HLA class I, or capable of specifically binding to a nucleic acid encoding the HA-1 mHag. In principle any type of method for specifically determining the presence of a particular expression product is suitable for the present invention and is, provided herewith. The HA-1 binding molecule may for instance be labeled, such as with green fluorescent protein or a radioactive label. A cell which is bound to an HA-1 binding molecule can also be detected with ELISA, affinity chromatography, etc. Of course, also provided is a non hematopoietic tumor cell comprising a molecule capable of specifically binding to an HA-1 mHag presented in the context of HLA class I, or capable of specifically binding to a nucleic acid encoding the HA-1 mHag.

Methods of the invention can be performed in vitro, however, in a preferred embodiment a method is performed in vivo. In vivo, a method of the invention can be used for the prevention and/or treatment of diseases caused by tumor cells. Provided is a method for at least in part inhibiting expansion of a tumor in an individual comprising providing the individual with a CTL specific for an HA-1 mHag presented in the context of HLA class I, or a functional equivalent of the CTL, the method characterized in that the tumor cell comprises a non-hematopoietic tumor cell presenting the HA-1 mHag in the context of the HLA class I. To obtain inhibition of expansion or even a reduction in tumor mass it is not required that all of the tumor cells express HA-1. Though non-HA-1 expressing cells are not eliminated by a method of the invention the removal of HA-1 expressing cells can still be relevant for treatment. Thus inhibition of expansion of tumor cells can also be achieved when only a part of the tumor cells express HA-1. A method of the invention may be performed in combination with other means of tumor cell removal. In a preferred embodiment the method is used to combat recurrence of tumor in situations of minimal residual disease. Of relevance for in vivo applications is the fact that normal hematopoietic cells also express HA-1. Thus, any method capable of specifically eliminating cells presenting HA-1 in the context of MHC class I, will in vivo also affect a hematopoietic system. To this end it is preferred to provide the individual with hematopoietic cells that are resistant to lysis by the CTL. This can be achieved in several ways. Preferably, an individual is transplanted with hematopoietic stem cells comprising a different or no HA-1 and/or different MHC class-I alleles. In a preferred embodiment the individual is transplanted with hemopoietic cells from an HA-1 negative donor. These cells can not be recognized by HA-1 specific CTL and can thus not be lysed by the T cells. In a preferred embodiment an individual is provided with stem cells that are negative for HA-1 or comprise a different HA-1 but the same MHC class-I compared to the tumor cell.

A method for elimination or killing of a tumor cell of the invention is particularly suited for the treatment of metastases, especially in the form of minimal residual disease, in particular liver metastases.

Cells for transplantation may be obtained directly from a donor. However, current culture technology allows significant expansion of cells in vitro, without detrimental effects on fitness and integrity of the cultured cells. Thus cells for transplantation and T-cells can be cultured in vitro if need be. One may also use T cells that are educated ex vivo to comprise T cell receptors capable of binding to HA-1 presented in the context of MHC class I. These educated T-cells can be expanded further ex vivo before providing them to an individual. A fully ex vivo approach toward education and expansion of the right T cells has the advantage that the cells can be analyzed and safety tested extensively before transplantation. A CTL of the invention is for instance generated by incubating T cells with dendritic cells comprising the MHC class-I and the HA-1. The dendritic cells are provided with the HA-1 by contacting with a peptide comprising the HA-1. Such systems also allow the generation of banks with extensively characterized and tested T cells with known specificities. Current and future technologies may be used to generate an HA-1 specific T-cell of the invention. For instance, current methods for the generation of such T-cell include introduction of the genetic information for the relevant T-cell receptor into cells that are already T cells or that can become T-cells. On the other hand functional equivalents of T cells of the invention are also possible. For instance, a suitable cloned T-cell receptor is introduced in so-called empty cells that are T-cells from which the relevant T-cell receptor is dysfunctional. Such T-cells do not express functional levels of native T-cell receptor chains and can thus not provide for chimeric T-cell receptors with unexpected specificities when provided with an HA-1 specific T cell receptor.

In one embodiment, T-cells and/or other hematopoietic cells provided to the individual comprise additional features. Such additional features can for instance be safety features, or additional (co)-stimulation features. Safety can be built in for instance using so-called suicide genes like Herpes Simplex Virus Thymidine Kinase (HSV-TK). Expression of HSV-TK is toxic for many cells when a pro-drug like gancyclovir is provided to the cells. A safety feature can be built in for a variety of reasons, one of which is a relatively simple way to down-regulate the number of grafted cells in the body in case of undesired effects of the grafted cells (GvHD and/or neoplasia). Additionally, other features can be built in, like, for instance, features to improve the anti-tumor effect of the grafted T-cells. This can be done by introducing co-stimulatory factors, cytokines and/or the encoding genes therefore into the T-cells. Thus, in a preferred embodiment a method of the invention is provided wherein the individual is provided with the CTL by providing the individual with a graft comprising hematopoietic cells of a donor.

In methods of the invention, T cells comprising a T cell receptor specific for HA-1 presented in the context of MHC class-I are being generated, isolated, manipulated and/or provided with additional features. T-cells obtained with a method of the invention are therefore also part of the invention. With the current pace of development in biological methods and knowledge, cells can be made into antigen specific T cells in an artificial way, for instance through manipulating programming genes. When such cells are made specific for HA-1 presented in the context of MHC class I than such cells are T-cells of the invention. T cells of the invention and equivalents thereof can be the basis for the preparation of medicaments for tumor cells. The invention thus also provides the use of an antigen specific T cell or an equivalent thereof comprising a specificity for HA-1 presented in the context of MHC class-I for the preparation of a medicament for the treatment of disease related at least in part to non-hematopoietic tumor cells. Provided is also a use of a molecule capable of specifically binding an HA-1 mHag in the context of HLA class I for preparing a medicament for the treatment of disease related at least in part to non-hematopoietic tumor cells. With a method of the invention, the growth of a non-hematopoietic tumor in an individual can be, at least in part, inhibited.

In an allogeneic SCT setting, donor lymphocyte infusion (DLI) therapy has been clearly shown curative for hematological malignancies (54, 55). However DLI therapy is associated with GvHD. To treat leukemia relapse after HLA matched, mHag HA-1 mismatched SCT with low risk of GvHD, we have previously developed ex vivo protocols for the generation of donor derived CTLs specific for the hematopoietic specific mHag HA-1 (56). These SCT donor derived HA-1 specific CTLs eliminate the HA-1 positive patient hematopoietic and leukemic cells, while HA-1 negative non-hematopoietic cells and tissues are spared. In the HLA identical allogeneic SCT setting for solid tumors, GvT reactivity has been demonstrated in small cohorts of patients with metastatic cancers, including breast cancer (46, 47, 57), melanomas (48), renal cell carcinomas (45) and ovarian carcinoma (49). From the present invention we know that at least part of this GvT reactivity is due to tumor specific polymorphic mHags such as HA-1. As in the leukemia transplant patients where residual leukemic tumor cells are present after high-dose chemotherapy, HA-1 directed immunotherapy is particularly warranted in cancer patients with minimal residual tumor cells who were shown to confer an increased risk for a later occurring relapse (58). Our observation of HA-1 expression on various types of non-hematological tumor cells, offers a novel target molecule for therapy. Similar to the cellular immunotherapy protocol for the treatment of relapsed leukemia, as described above, adoptive immunotherapy with donor derived HA-1 CTLs in combination with SCT is an attractive alternative treatment of non-hematopoietic tumors, preferably, solid tumors. Because of the hematopoietic expression of the HA-1 gene, the patient's hematopoiesis will be at least partly eliminated and needs reconstitution from donor SC or equivalents thereof. Based on HA-1 restricted expression on malignant non-hematopoietic tissues, HA-1 cellular therapy is specific, with no foreseen damage to normal tissues and cells. The above mentioned HA-1 based immunotherapy capitalizes on expression of HLA-A2 and/or HLA-B60 ligands for HA-1 CTL recognition, as is described below for HLA-B60, and the HA-1^{H} allele of the HA-1 locus. Since there are many different HLA-molecules it is expected that at least some other HLA molecules are also capable of presenting HA-1. Methods of the invention are also suitable for these other HLA molecules. The HA-1^{H} phenotype frequency is 69% in the HLA-A2 positive population (7). To our knowledge this is the first example of a constitutive hematopoietic specific gene that can function as a universal tumor specific antigen, with no significant expression on its non-malignant counterparts. The significance of the polymorphic mHag HA-1 for cancer therapy is underscored by the known HA-1 immunogenic functional membrane expression and adequate CTL recognition.

It may also be relevant to counteract an immune response against HA-1 in an individual. This may for instance be the case when the immune response is no longer needed or when rescue of autologous cells is desired, for instance in case of inadvertent negative effects of foreign HA-1 specific T-cells. An infused cell can for instance become neoplastic, or otherwise deregulated in its cytokine excretion or responsiveness. Thus an efficient way of counteracting an immune response against HA-1 is useful for a variety of reasons. Some ways to counteract are given elsewhere in this application. Here another way of counteracting is provided. The present invention in one aspect provides a method to modulate mHag HA-1 response by providing an antagonistic peptide. A peptide can be provided to an MHC-I expressing cell in many ways, for instance through providing the peptide, or the encoding nucleic acid. The peptide is designed such that it comprises a sufficiently strong affinity for the HLA-molecule that HA-1 peptide is effectively prevented from associating with HLA, thereby at least in part reducing the capability of the HA-1 specific immune response to attack the cell.

In yet another aspect, the invention provides a method for the treatment of an individual suffering from or at risk of suffering from a non-hematopoietic tumor comprising inducing and/or enhancing in the individual an immune response against HA-1 presented in the context of HLA class I. In one embodiment the immune response is induced and/or enhanced by administering a CTL specific for HA-1 presented in the correct context. In another embodiment the immune response is induced and/or enhanced by vaccinating the individual with a (poly)peptide comprising HA-1 antigen. Vaccinations may be performed using any method for vaccination against a peptide known in the art. A preferred means of vaccination comprises the so-called string of beads method of vaccination wherein several different peptides are incorporated into a proteinaceous molecule. When HA-1 antigen is provided in the context of a larger molecule it is preferred that the peptide comprising the HA-1 antigen is flanked at least on one side but preferably on both sides by appropriate processing sites to allow cutting of the HA-1 antigen and the transport of the antigen to the relevant site and the association of the antigen with the appropriate MHC-I molecule. Vaccinations can of course also be performed using other methods known in the art. Such methods preferably comprise MHC tetramers. Vaccinations may be performed in the traditional sense or vaccination may be performed using artificial antigen presenting moieties in the form of liposomes comprising such MHC presenting the relevant HA-1 antigen. Vaccinations may of course comprise any suitable type of adjuvant. Preferably, the adjuvant comprises CpG rich genes. This adjuvant is particularly preferred when vaccination is performed with nucleic acid coding for an expressible HA-1 antigen.

In a preferred embodiment, an individual suffering from, or at risk of suffering from, a non-hematopoietic tumor is provided with hematopoietic stem cells from a donor, and is vaccinated with HA-1 antigen. The donor cells preferably express no, or a different, HA-1. According to the invention, vaccination with HA-1 at least partly solves the problem that allogeneic stem cell transplantation often results in Graft versus Host reactivity without tumor specificity. Vaccination with HA-1 enhances the specificity of the Graft versus Host reaction to such extent that GvHD is at least partly diminished. In one embodiment a method of the invention is therefore provided which involves a combination of stem cell transplantation and HA-1 vaccination. Preferably, the individual suffering from, or at risk of suffering from, a non-hematopoietic tumor is vaccinated. The vaccination is preferably performed after the individual has been provided with donor stem cells. It is however also possible to vaccinate a donor with HA-1 and to subsequently provide an individual with donor hematopoietic cells, although vaccination of a healthy donor is usually not a first method of choice.

Various adjuvants can be used for the vaccination, as described above. For instance, donor dendritic cells comprising HA-1 can be used. Alternatively, GM-CSF, or CpGs, + HA-1 peptide can be used. In the art, many alternative ways of vaccination are known that can be used in a method of the invention. in one embodiment, stem cell transplantation is performed by directly providing donor cells to the individual. Alternatively, adoptive immunotherapy can be used.

As described above, in general, peptides presented in the context of HLA vary in length from about 7 to about 15 amino acid residues, and a polypeptide can be enzymatically processed to a peptide of such length. A peptide comprising HA-1 antigen provided by the invention typically is at least 7 amino acids in length, but preferably at least 8 or 9 amino acids. The upper length of a peptide provided by the invention is typically no more than 15 amino acids, but preferably no more than about 11 to 13 amino acids in length. A peptide provided by the invention contains the necessary anchoring residues for presentation in the groove of the relevant HLA molecule. An immunogenic polypeptide provided by the invention comprises a 7-15 amino acid long peptide, optionally flanked by appropriate enzymatic cleavage sites allowing processing of the polypeptide. Presentation of the HA-1 antigen by MHC-I can occur in various ways depending on the particular type of MHC-I. Different HLA molecules behave differently in their capacity to present a peptide. In the present invention HA-1^{H} antigen can be presented by different HLA molecules. In case of HLA-A2 the peptide presented comprises the sequence VLHDDLLEA (SEQ ID NO:______). When the HLA molecule is HLA-B60 the HA-1^{H} antigen comprises a sequence that is shifted slightly when compared to the sequence presented by HLA-A2. This is described in more detail below. However, the polymorphism is of course still present in the peptide presented by HLA-B60. Thus the HA-1 antigen may comprise any peptide capable of being presented by an MHC-I or for that matter MHC-II molecule provided that it comprises the relevant polymorphism.

In one embodiment the peptides and other molecules according to the invention find their utility in that they induce and/or enhance an immune induced elimination of non-hematopoietic tumor cells. Since the hematopoietic cells of an HA-1 positive recipient also express HA-1, it is preferred that the individual wherein an immune response against HA-1 in the context of HLA is induced and/or enhanced is provided with HA-1 negative hematopoietic stem cells. The above-mentioned HA-1 antigen containing (poly)peptides are used to prepare therapeutic agents capable of eliminating a subset of cells, directly or indirectly, especially tumor cells of non-hematopoietic origin. This can be illustrated by the following examples, which refer to leukemia related therapeutic agents.

An HA-1 positive, non-hematopoietic tumor baring recipient (in bone marrow transplantation) is subjected to an additional pre-bone marrow transplant conditioning regime. This means that an agent which specifically recognizes a (poly)peptide according to the invention (an HA-1 comprising (poly)peptide) as presented selectively on hematopoietic cells, which agent induces elimination of the cells presenting the peptide, is administered to the recipient before transplantation. This agent will eliminate all (residual) tumor cells and cells of hematopoietic origin. Such agents include but are not limited to T cells (which are, for instance, tailor made ex vivo by pulsing with the peptides provided by the invention, and optionally provided with a suicide gene) and/or antibodies coupled to toxic moieties.

An HA-1 negative donor for bone marrow transplantation can be vaccinated with a peptide according to the invention, an HA-1 peptide. Upon transplantation to an HA-1 positive recipient, the donor's immune system can eliminate any residual or recurrent HA-1 peptide presenting cells in the recipient which are of course leukemic.

This is another example of tailor-made adoptive immunotherapy provided by the invention. A transplanted HA-1 positive recipient, transplanted with HA-1 negative (or for that matter HA-1 positive) bone marrow and suffering from recurrent disease (relapse), i.e., HA-1 positive tumor cells, can be treated with an agent as above which specifically recognizes a peptide according to the invention (an HA-1 peptide) as presented on hematopoietic cells, which agent induces elimination of the cells presenting the peptide. In case of HA-1 positive bone marrow being transplanted to the HA-1 positive recipient, it is still very important (in case of recurrent disease) to eliminate all HA-1 positive cells even though this includes the transplanted material, because otherwise the HA-1 positive tumor will kill the recipient. To avoid the latter case, the patient can be re-transplanted, if necessary. In such therapy protocols it is possible to first employ adoptive immunotherapy with agents (cells, antibodies, etc.) which specifically recognize and eliminate specific peptide expressing cells (e.g., tumor cells) that need to be destroyed, after which in a second phase the patient is reconstituted with BMT cells replacing the killed cells. The invention thus provides additional (or even substituting) protocols to other therapeutic measures such as radiation.

A CTL capable of specifically killing a cell presenting HA-1 in the context of a suitable HLA class I molecule is to be an HA-1 specific CTL, even in cases wherein the CTL was raised (educated) against a different peptide.

A (poly)peptide is to comprise an HA-1 antigen when a suitable part of the (poly)peptide is recognized by the aforementioned HA-1 specific CTL when the part is presented in the context of a suitable HLA molecule.

Now that the invention discloses that non-hematopoietic tumor cells express HA-1 it is possible to use this information for instance in developing diagnostic tools. Considering that normal non-hematopoietic cells do not express HA-1, it is possible to discriminate between a tumor non-hematopoietic cell and a normal non-hematopoietic cell, on the basis of HA-1 gene expression. This can be done on the protein (peptide) level and/or on the nucleic acid level. The invention therefore further provides a method for marking a non-hematopoietic tumor cell comprising incubating the cell with a molecule capable of specifically binding to an HA-1 mHag presented in the context of HLA class I, or capable of specifically binding to a nucleic acid encoding the HA-1 mHag. Means and methods for determining the presence of HA-1 polypeptide or mRNA in a cell are well known to the person skilled in the art. Examples of detection methods are described in PCT International Publication WO 99/05313, which is herein incorporated by reference. These methods may be combined with other detection and/or cell type characterization methods (for instance for expression products of other genes or microscopy) to exclude the presence of HA-1 expressing hemopoietic cells. A cell comprising the molecule capable of specifically binding to an HA-1 mHag presented in the context of HLA class I, or capable of specifically binding to a nucleic acid encoding the HA-1 mHag, is also part of the invention.

As described above, one aspect of the present invention provides a (poly)peptide which is functionally presented to the immune system in the context of an HLA-A2.1 molecule. The (poly)peptide allows for HA-1-specific immunotherapy in patients that are positive for HLA-A2.1. However, a significant part of the population is negative for HLA-A2.1 and, hence, cannot be treated with the (poly)peptide or with binding moieties recognizing the (poly)peptide in the context of HLA-A2.1. Clearly, a need exists for further HA-1 peptides that are capable of associating with other HLA molecules. Particularly, there is a need for further HA-1 peptides that are capable of associating with other HLA class I molecules. With such HA-1 peptides, HA-1-specific immunotherapy can be extended to patients that are negative for HLA-A2.1.

We have found such further HA-1 peptides. A novel HLA-B60 restricted T cell epitope of HA-1 is provided comprising the sequence KECVLXDDL (SEQ ID NO:______). X represents either a histidine or an arginine residue. With the HLA-B60 restricted HA-1 epitope it has become possible to enlarge the patient population for HA-1 specific immunotherapy. Patients that are negative for HLA-A2.1 but positive for HLA-B60 can now be subjected to a method of the invention.

In one aspect, the invention therefore provides a peptide constituting a T-cell epitope obtainable from the minor Histocompatibility antigen HA-1 comprising the sequence KECVLXDDL (SEQ ID NO:______) or a derivative thereof having similar functional or immunological properties, wherein X represents a histidine or an arginine residue. The way these sequences are obtained is described in the examples. Nonameric as well as decameric peptides have been found that show strong binding capacity to HLA-B60 molecules. Especially, the nonameric and decameric HA-1^{H/R} peptides KECVLHDDL (SEQ ID NO:______), KECVLRDDL (SEQ ID NO:______), KECVLHDDLL (SEQ ID NO:______) and KECVLRDDLL (SEQ ID NO:______) show strong binding to HLA-B60 molecules. Hence, in one embodiment a peptide or a derivative of the invention is provided, wherein the peptide comprises the sequence KECVLXDDLL (SEQ ID NO:______). As used herein, an X in a sequence of a peptide of the invention represents a histidine or an arginine residue.

As described elsewhere herein, once a sequence of the invention is known, peptides comprising such sequence can easily be made synthetically. Methods for generating peptides synthetically are well known in the art. It is also within the skill of the art to arrive at analogs or derivatives of a peptide of the invention. The analogs or derivatives can for instance be generated using conservative substitution. This means a substitution of one amino acid with another amino acid with generally similar properties (size, hydrophobicity, etc), such that the overall functioning is likely not to be seriously affected.

The analog or derivative has the same or at least similar functional or immunological properties and/or activity. “The same or at least similar functional or immunological properties and/or activity” means herein that at least one of the properties and/or activity is the same in kind, not necessarily in amount, as compared to the functional or immunological properties and/or activity of the peptide the analog or derivative is derived from. Preferably, the analog or derivative has essentially maintained most of the functional or immunological properties and/or activity of the peptide in kind, not necessarily in amount. An analog or derivative of the above-mentioned HLA-B60 restricted HA-1 epitope of the invention is preferably as well capable of binding an HLA-B60 molecule.

The invention provides a (poly) peptide which can be functionally presented to the immune system in the context of an HLA-B60 molecule. In general peptides presented in such a context vary in length from about 7 to about 15 amino acid residues, and a polypeptide can be enzymatically processed to a peptide of such length. A peptide provided by the invention typically is at least 7 amino acids in length but preferably at least 8 or 9 amino acids. The upper length of a peptide provided by the invention is no more than about 15 amino acids, but preferably no more than about 13 or 11 amino acids in length. A peptide provided by the invention contains the necessary anchoring residues for presentation in the groove of the HLA-B60 molecule.

Thus, in one aspect an immunogenic polypeptide obtainable from the minor Histocompatibility antigen HA-1 comprising the sequence KECVLXDDL (SEQ ID NO:______) or a derivative thereof having similar functional or immunological properties is provided. In one embodiment the polypeptide comprises the sequence KECVLXDDLL (SEQ ID NO:______).

An immunogenic polypeptide provided by the invention comprises a 7-15 amino acid long peptide of the invention, optionally flanked by appropriate enzymatic cleavage sites allowing processing of the polypeptide.

In a preferred embodiment of the invention, a peptide comprising the sequence KECVLHDDL (SEQ ID NO:______) or KECVLHDDLL (SEQ ID NO:______) is provided, which induces lysis of the cell presenting it at a very low concentration of peptide present. This does not imply that peptides inducing lysis at higher concentrations are not suitable. This will for a large part depend on the application and on other properties of the peptides, which were not all testable within the scope of the present invention.

The peptides, derivatives and analogs of the invention find their utility in that they are used to induce tolerance of the donor immune system in HA-1 negative donors, so that residual peripheral blood lymphocytes in the eventually transplanted organ or the bone marrow, as it may be, do not respond to host HA-1 material in an HA-1 positive recipient. In this way GvHD is prevented or mitigated. On the other hand, tolerance is induced in HA-1 negative recipients in basically the same way, so that upon receipt of an organ or bone marrow from an HA-1 positive donor no rejection on the basis of the HA-1 material occurs. For tolerance induction, very small doses are given repeatedly, for instance intravenously, but other routes of administration may very well be suitable too. Another possibility is the repeated oral administration of high doses of the peptides. The peptides, derivatives and/or analogs thereof are given alone, in combination with other peptides, as part of larger molecules, or coupled to carrier materials in any suitable excipients.

Further applications of a peptide, derivative and/or analog of the invention lie in the prophylactic administration to transplanted individuals to prevent GvHD. This is done with either agonists, possibly in combination with an adjuvant, or with antagonists which block the responsible cells. This can be done with or without the concomitant administration of for instance TCR derived peptide sequences or cytokines. Furthermore, a peptide, derivative and/or analog of the invention is used to prepare a therapeutic agent capable of eliminating a subset of cells, directly or indirectly, especially cells of hematopoietic origin and/or tumor cells, as described above for HLA-A2.1 restricted peptides of the invention comprising the sequence VLXDDLLEA (SEQ ID NO:______). The applications for an HLA-A2.1 restricted peptide of the invention, particularly suitable for HLA-A2.1 positive individuals, are similar to the applications of an HLA-B60 restricted peptide of the invention which are particularly suitable for HLA-B60 positive individuals. HA-1-specific immunotherapy can now be applied to HLA-A2.1 positive as well as HLA-B60 positive patients.

As described elsewhere, aberrant cells of an HA-1 positive patient, such as, for example, leukemic cells and/or tumor cells, are eliminated by administration to the patient of an agent specifically recognizing a peptide of the invention, the agent being capable of inducing elimination of cells presenting the peptide. AN HA-1 negative donor for bone marrow transplantation is vaccinated with a peptide of the invention in order to enhance elimination by the donor's immune system of any HA-1 peptide presenting cells in a recipient. Other therapeutic applications of a peptide of the invention include induction of tolerance to HA-1 proteins in HA-1 related (auto)immune diseases. On the other hand, they are used in vaccines, for instance in HA-1 related (auto)immune diseases, tumor related diseases, etcetera.

The invention thus provides a pharmaceutical formulation and/or a vaccine comprising a peptide or a polypeptide of the invention. A peptide or polypeptide of the invention for use as a medicament is also herewith provided. The medicament is preferably capable of inducing tolerance for transplants to prevent rejection and/or GvHD. In another preferred embodiment the medicament is capable of, at least in part, treating (auto)immune disease.

According to the present invention, HA-1 is expressed in a functional way on the membrane of tumor cells, including tumor cells of non-hematopoietic origin. Hence, a peptide of the invention is also particularly suitable for inducing a (specific) immune response against a tumor. A use of a peptide or polypeptide of the invention in the preparation of a medicament for treatment of a disease that is at least in part related to tumor cells is therefore also provided. In a preferred embodiment, the tumor cells comprise non-hematopoietic tumor cells.

Preferably, the peptide or polypeptide of the invention comprises a peptide constituting a T-cell epitope obtainable from HA-1 comprising the sequence KECVLXDDL (SEQ ID NO:______) or KECVLXDDLL (SEQ ID NO:______) or a derivative thereof having similar functional or immunological properties.

Now that a peptide comprising an HLA-B60 restricted HA-1 epitope is provided, one can, at least in part, eliminate a cell presenting the peptide. The cell preferably comprises a hematopoietic cell (more preferably a neoplastic hematopoietic cell) and/or a tumor cell (most preferably a non-hematopoietic tumor cell).

HA-1 was reported to be expressed in hematopoietic cells only. According to the invention, however, HA-1 is as well expressed in a functional way on the membrane of tumor cells of non-hematopoietic origin. A binding moiety capable of specifically recognizing a cell presenting a peptide of the invention is used to bind and eliminate the cell. One embodiment of the invention therefore provides a method for the elimination of a (neoplastic) hematopoietic cell and/or a tumor cell presenting a peptide or polypeptide of the invention in the context of HLA-B60, whereby elimination is induced directly or indirectly by specific recognition of the (poly)peptide in the context.

The binding moiety preferably comprises a cytotoxic T lymphocyte. As described elsewhere, mHag peptide CTLs can be generated ex vivo from mHag negative donors. Peptide-specific CTL clones from an HLA-B60 positive, HA-1 negative donor are for instance generated by pulsing autologous APCs with mHag HA-1 related synthetic peptide. Proliferating clones are expanded and tested for specific cytotoxic activity.

In one aspect, the invention provides a method for killing a hematopoietic cell and/or tumor cell functionally expressing an HA-1 mHag comprising a peptide or polypeptide of the invention in the context of HLA-B60, comprising incubating the cell with a cytotoxic T lymphocyte (CTL) specific for the mHag presented in the context or incubating the cell with a functional equivalent of the CTL. Preferably, the tumor cell comprises a non-hematopoietic tumor cell.

A CTL capable of specifically killing a cell presenting HA-1 in the context of HLA-B60 is said to be specific for an HA-1 presented in the context, even in cases wherein the CTL was raised (educated) against a different peptide.

Now that the invention discloses peptides comprising an HLA-B60 restricted T cell epitope of HA-i, it is possible to use the peptides in developing diagnostic tools. Since normal hematopoietic cells do not express HA-1, it is possible to discriminate between non-hematopoietic cells on the one hand and hematopoietic cells and/or tumor cells on the other. It is possible to discriminate between those cells on the basis of HA-1 gene expression. This can be done on the protein (peptide) level and/or on the nucleic acid level. The invention therefore provides a method for marking a hematopoietic cell and/or a tumor cell comprising incubating the cell with a molecule capable of specifically binding to an HA-1 mHag comprising a peptide or polypeptide of the invention presented in the context of HLA-B60, or capable of specifically binding to a nucleic acid encoding the HA-1 mHag. Preferably, the tumor cell comprises a non-hematopoietic tumor cell.

As described above, means and methods for determining the presence of HA-1 polypeptide or mRNA in a cell, such as for instance detection methods described in PCT International publication WO 99/05313, are well known to the person skilled in the art. These methods may be combined with other detection and/or cell type characterization methods (for instance for expression products of other genes or microscopy) to exclude the presence of HA-1 expressing hematopoietic cells. A cell comprising the molecule capable of specifically binding to an HA-1 mHag presented in the context of HLA class I, or capable of specifically binding to a nucleic acid encoding the HA-1 mHag, is also part of the invention.

It is often advantageous to test whether an individual expresses HA-1. For instance, a donor and recipient of bone marrow or organ transplantation are preferably tested in order to determine whether they are mHag-matched. As another example, tumor cells obtained from an individual can be screened for HA-1 expression. A CTL specific for HA-1 in the context of HLA class I can be used for determining whether a cell expresses functional levels of HA-1 in the context of HLA Class I. In one embodiment of the invention, a CTL specific for a peptide of the invention in the context of HLA-B60 is used. The invention therefore further provides a method for determining whether a cell expresses functional levels of an HA-1 mHag comprising a peptide or polypeptide according to the invention in the context of HLA-B60, comprising incubating the cell with a cytotoxic T lymphocyte (CTL) specific for the HA-1 mHag presented in the context and determining whether the cell and/or the CTL is affected.

There are several ways to determine whether the cell or the CTL is specifically affected by the incubation. One typically uses target cell killing to determine specific recognition by CTL. Other ways are however possible as well, such as detection of a gene expression pattern characteristic for CTL mediated lysis in the CTL or target cell.

The invention also provides an analog of a peptide of the invention which is an antagonist for the activity of T cells recognizing the peptide (preferably in the context of HLA-B60). Such an analog is obtained using methods and tests known in the art. Furthermore, the invention provides a method for the generation of antibodies, T cell receptors, anti-idiotypic B-cells or T-cells, comprising the step of immunization of a (preferably HLA-B60 positive) mammal with a peptide or a polypeptide of the invention. Antibodies, T-cell receptors, B-cells or T-cells obtainable by the method are also herewith provided. Dose ranges of peptides, antibodies and/or other molecules according to the invention to be used in therapeutic applications are designed on the basis of rising dose studies in clinical trials for which rigorous protocol requirements exist.

As described elsewhere herein, T cells can be educated in vivo as well as ex vivo to comprise T cell receptors capable of binding to a minor antigen presented in the context of MHC class I. Now that a peptide comprising an HLA-B60 restricted HA-1 epitope is provided herein, cytotoxic T cells against the peptide/epitope are also generated. A CTL of the invention is generated by incubating a T cell with an antigen presenting cell (APC), preferably a dendritic cell, comprising the peptide. The APC most preferably comprises HLA-B60. The APCs may be provided with an HA-1 epitope of the invention by contacting the cells with a peptide comprising the HA-1 epitope.

In one embodiment, the invention thus provides a method for the generation of a cytotoxic T-cell against a minor antigen, comprising contacting a hematopoietic cell with a peptide or a polypeptide of the invention. The minor antigen preferably comprises HA1. More preferably, the hematopoietic cell is contacted with the peptide or polypeptide in the context of HLA-B60.

These educated T-cells can be expanded further ex vivo before providing them to an individual. A fully ex vivo approach toward education and expansion of the right T cells has the advantage that the cells can be analyzed and safety tested extensively before transplantation. Hence, a T-cell of the invention is preferably capable of expansion.

In a preferred embodiment, the hematopoietic cell itself is negative for the minor antigen. This allows exposure of a T cell to a peptide of the invention without exposure to additional epitopes of the minor antigen.

As described elsewhere, current and future technologies may be used to generate an HA-1 specific T-cell of the invention, and functional equivalents of T cells of the invention are also part of the invention.

The invention also includes a T-cell, or derivative or active fragment thereof, specifically directed against a peptide of the invention. The T cell is preferably a cytotoxic T cell. In one embodiment, the T-cell, derivative or active fragment may be obtained with a method of the invention. Preferably, the T-cell, derivative or active fragment is specifically directed against a peptide of the invention in the context of an HLA-B60 molecule.

With the current pace of development in biological methods and knowledge cells can be made into antigen specific T cells in an artificial way, for instance through manipulating programming genes. When such cells are made specific for HA-1 presented in the context of MHC class I than such cells are T-cells of the invention.

In one embodiment, T-cells of the invention are provided comprising additional features. Such additional features for instance comprise safety features, or additional (co)-stimulation features. Safety is for instance built in using so-called suicide genes. Additionally, other features can be built in, like for instance features to improve anti-tumor effect of grafted T-cells.

Once a CTL specific for HA-1 has been generated, it can be used to counteract expansion of a tumor cell. Now that the invention provides methods for generating CTLs specific for an HA-1 peptide in the context of HLA-B60, tumor cells in HLA-B60 positive patients are counteracted as well. As explained elsewhere, to obtain inhibition of expansion or even a reduction in tumor mass it is not required that all of the tumor cells express HA-1. Inhibition of expansion of tumor cells can also be achieved when only a part of the tumor cells express HA-1.

One aspect of the invention therefore provides a method for at least in part inhibiting expansion of a tumor cell in an individual comprising providing the individual with a CTL specific for an HA-1 mHag comprising a peptide or polypeptide of the invention presented in the context of HLA-B60, or a functional equivalent of the CTL. In a preferred embodiment, the individual is provided with the CTL by providing the individual with a graft comprising hematopoietic cells of a donor.

A CTL specific for an HA-1 mHag can be generated ex vivo. However, the CTL can be generated in vivo as well. A peptide of the invention is administered to an HLA-B60 positive individual that comprises a mismatch for HA-1. In one application, the individual comprises a donor of lymphocytes. After generation of HA-1 directed T lymphocytes, the lymphocytes can be used for counteracting a tumor, preferably a non-hematopoietic tumor, in an HA-1 positive patient. The invention thus provides a use of an HA-1 antigen comprising a peptide or polypeptide of the invention for inducing and/or enhancing the generation of HA-1 specific cytotoxic lymphocytes in an HA-1 negative donor of lymphocytes, wherein the generated lymphocytes are used for the preparation of a medicament for the treatment of a disease that is at least in part related to tumor cells.

Alternatively, A CTL specific for an HA-1 mHag can be generated in vivo by administration of a tumor cell expressing the HA-1 mHag in the context of HLA-B60 to an HLA-B60 positive individual that comprises a mismatch for HA-1. Upon administration, the individual will produce HA-1 directed CTL. A method of the invention is preferably performed in a non-human animal in order to produce the desired CTLs. Alternatively, a method of the invention is used for vaccination purposes.

One embodiment of the invention thus provides a method for generating a CTL capable of binding to an HA-1 mHag comprising a peptide or polypeptide of the invention, presented in the context of HLA-B60, comprising the step of administering to an individual that comprises a mismatch for the HA-1 mHag, a tumor cell expressing the HA-1 mHag presented in the context. The tumor cell preferably comprises a non-hematopoietic tumor cell.

A molecule capable of specifically binding an HA-1 mHag in the context of HLA-B60, such as the above-mentioned CTLs, is particularly suitable for eliminating tumor cells. Hence, a medicament is generated comprising the binding molecule. A use of a molecule capable of specifically binding an HA-1 mHag comprising a peptide or polypeptide of the invention in the context of HLA-B60 for the preparation of a medicament for the treatment of a disease that is at least in part related to tumor cells is therefore also provided herewith. Preferably, the tumor cells comprise non-hematopoietic tumor cells.

### BRIEF DESCRIPTION OF THE FIGURES

FIG. 1. Reconstitution of HA-1 with HPLC fractionated peptides eluted from HLA-A2.1 molecules in a ⁵¹Cr-release assay with mH HA-1 specific T cell clone 3HA15. a. Peptides were eluted from 90.10⁹ HA-1 and HLA-A2.1 positive Rp cells and separated using reverse phase HPLC with HFBA as organic modifier. b. Fraction 24 of the first HPLC dimension that contained HA-1 activity was further fractionated by reverse phase HPLC with TFA as organic modifier. c. HA-1 containing fraction 27 of the second gradient was further chromatographed with a third shallower gradient consisting of 0.1% acetonitrile/min. Background lysis of T2 by the CTL in the absence of any peptides was in a 3%, in b and c 0%. Positive control lysis was in a 99%, in b 74% and in c 66%. d. Determination of candidate HA-1 peptides. HPLC fraction 33 from the separation in FIG. 1c. was chromatographed with an on-line microcapillary column effluent splitter and analyzed by electrospray ionization mass spectrometry and a ⁵¹Cr-release assay. HA-1 reconstituting activity as percent specific release was compared with the abundance of peptide candidates measured as ion current.

FIG. 2. Sequencing of mH HA-1 peptide by tandem mass spectrometry. a.

Collision activation dissociation mass spectrum of peptide candidate with m/z of 513. b. Reconstitution assay with different concentrations of synthetic mH HA-1 peptide with three HA-1 specific T cell clones, 3HA15, clone 15 and 5W38. Background lysis of T2 by the CTL in the absence of any peptide was for 3HA15 4%, for clone 15 10% and for 5W38 2%. Positive control lysis was for 3HA15 46%, for clone 15 47% and 5W38 48%.

FIG. 3. KIAA0223 polymorphism exactly correlated with mH antigen HA-1 phenotype. a. The HA-1 region of KIAA0223 was sequenced in an HA-1 mH antigen typed family. 6 PCR products of each family member were sequenced. Family members 00, 07 and 09 expressed the HA-1^{R} in all 6 PCR products. Family member 01 expressed the HA-1^{H} allele in 2 PCR products and the HA-1^{R} allele in 4 PCR products. Family member 02 expressed the HA-1^{H} allele in 3 PCR products and the HA-1^{R} allele in 3 PCR products. Family member 08 expressed the HA-1^{H} allele in 4 PCR products and the HA-1^{R} allele in 2 PCR products b. HA-1 allele specific PCR reaction in an HA-1 mH antigen typed family correlated exactly with the HA-1 phenotype. The sizes of the resulting PCR products were consistent with the expected sizes deduced from the cDNA sequence. c. Transfection of the HA-1^{H} allele of KIAA0223 leads to recognition by mH HA-1 specific T cells. The HA-1^{H} and the HA-1^{R} coding sequence of KIAA0223 were together with HLA-A2.1 transfected into Hela cells. After 3 days the HA-1 specific CTL clones 5W38 and 3HA15 were added and after the 24 hours TNFa release was measured in the supernatant. The clone Q66.9 is specific for the influenza matrix peptide 58-66. No TNFa production was observed after transfection of the pcDNA3.1 (+) vector alone (results not shown).

FIG. 4. a. Binding of HA-1^{H} and HA-1^{R} peptides to HLA-A2.1. The binding of HA-1^{H} and HA-1^{R} peptides were assayed for their ability to inhibit the binding of fluorescent peptide FLPSDCFPSV to recombinant HLA-A2.1 and b2-microglobulin in a cell free peptide binding assay. One representative experiment is shown. The IC50 is determined on the results of 4 experiments and was 30 nM for VLHDDLLEA (SEQ ID NO:______) and 365 nM for VLRDDLLEA (SEQ ID NO:______). b. Reconstitution assay with different concentrations of synthetic HA-1^{R} peptide with HA-1 specific T cells. The HA-1^{R} peptide was titrated and preincubated with T2 cells. Three HA-1 specific T cell clones, 5W38, 3HA15 and clone 15 were added and a 4 hr ⁵¹Cr-release assay was performed. Background lysis of T2 by the CTL in the absence of any peptide was for 3HA15 4%, for clone 15 10% and for 5W38 2%. Positive control lysis was for 3HA15 46%, for clone 15 47% and 5W38 48%.

FIG. 5. Cytotoxic T cell activity against peptide pulsed and mHag positive target cells by two ex vivo induced HA-1 (a, b) and two ex vivo induced HA-2 specific CTLs (c, d). CTLs shown in a,c,d are induced using PBDC, whereas CTLs shown in b are induced using BMDC. Target cells: autologous PHA blasts (⋄); autologous PHA blasts pulsed with peptide (?); EBV-LCL positive for HA-1 (n=4) or HA-2(n=3) (?); EBV-LCL negative for HA-1 (n=3) or HA-2(n=3) (?); HA-1 or HA-2 negative EBV-LCL pulsed with HA-1 or HA-2 peptide (•).

FIG. 6. Hematopoietic cell restricted cytolysis mediated by in vivo (a,c) and ex vivo (b,d) induced HA-1 (a,b) and HA-2 (c,d) specific CTLs. All target cells were derived from the same HLA-A2+, HA-1+, HA-2+blood donor. Target cells: PHA blasts (?); fibroblasts (?); Fibroblasts cultured with IFN-?+ TNF-a (both 300 U/ml) (•); Fibroblasts cultured with IFN-? plus TNF-a and pulsed with 10 μg/ml peptide (?).

FIG. 7. Lysis of HA-1⁺ (a,b,c) or HA-2⁺ (d,e,f) positive leukemic cells by in vivo (b,e) and ex vivo (c,f) induced HA-1 and HA-2 specific CTLs. Lysis of target cells by control HLA-A2 specific CTL clone is shown in a and d. Target cells: HA-1 or HA-2 negative EBV-LCL (_), HA-1 or HA-2 positive EBV-LCL (?), Leukemic cells positive for HA-1 (n=4) or HA-2 (n=3) (?), HA-1 or HA-2 positive leukemic cells cultured with IFN-?+ TNF-a (•).

FIG. 8. HA-1 gene expression in hematopoietic and non-hematopoietic cells. The relative HA-1 gene expression levels were determined by a calibration function generated from RNA of the HA-1 positive KG-1 cell line. Cells of hematopoietic origin tested were: *PBMCs (n=3), Dendritic cells (n=6), + Langerhans cells (n=2), £ EBV-LCLs (n=5), uPHA blasts (n=6), ⁻ Mast cell lines (n=3), Í Monocytes (n=4), [symbol] Thymocytes (n=Cells of non-hematopoietic origin tested were: [symbol] Keratinocytes (n=5), ¢ Fibroblasts (n=2), − PTECs (n=3), r HUVECs (n=3), s Melanocytes (n=3), two SV40 immortalized breast cell lines: ⁻ HaCat and [symbol] HBL-100.

FIG. 9: HA-1 and CD45 expression of micro-dissected tissue samples A and B. Micro-dissection of primary tumor (adenocarcinoma of the lung) and normal breast gland, respectively. C. Three to six areas from a tumor samples (about 10,000-50,000 μm2 of a 5 μm section) were individually analyzed by gene specific PCR for HA-1 (primer HA-1 (II)) and CD45. The same was done using pooled cDNA from several milk ducts (in total 60,000 μm2 each) from normal breast tissue of three different donors (controls 1-3). Lane numbering indicates the different micro-dissected areas, PN=patient number. Arrows point to tumor areas without contaminating hematopoietic cells but positive HA-1 signal.

FIG. 10: Isolation and gene expression analysis of single disseminated cancer cells or small tumor cell clusters. A. Three-cell cluster (PN5-C4) after micromanipulator-assisted isolation from a cell suspension of a lymph node preparation. All cells of the cluster are intensively stained by the EpCAM antibody. B. Gene expression profiling on cDNA array of isolated tumor cells. HA-1 expression after standard RT-PCR is given in the first line. The grey shades represent the signal intensity (from light grey=weak signal to black=strong signal).

FIG. 11: HA-1 expression of disseminated cancer cells. Cells positive for the HA-1 (11) primer pair were digested with Hinf I, blotted and hybridized with the respective probe. M=size marker, A=undigested PCR product, B=Hinf I digested product; lane 1=PN12-C1; lane 2=PN4-C1; lane 3=PN3-C1; lane 4=PN5-C1; lane S=PN6-C5; lane 6=PN2-C1; +=HT29 for HA-1 and normal bone marrow for CD45.

FIG. 12. CGH profile of cell PN3-C1. Each chromosome is represented by its ideogram and numbered. Deletions are marked with a red bar (e.g. loss of chr.13) at the left and gains with a green bar (e.g. gain of chr. 8q) at the right side of the chromosome symbol.

FIG. 13. Binding of HA-1^{H/R} peptides to HLA-A3. The results are expressed as the percentage inhibition of the HLA binding of the 150 nM fluorescent reference peptide by the indicated peptides added at serial dilutions (see material and methods). Curves were fitted by nonlinear regression and one site binding equation. The IC₅₀ value of the HLA-A3 binder positive control peptide KQSSKALQR (9) was 9.4 μM.

FIG. 14. Efficient binding of HA-1^{H/R} peptides to HLA-B60. The results are expressed as the percentage inhibition of the HLA binding of the 150 nM reference peptide by the indicated peptides added at serial dilutions (see material and methods). Curves were fitted by nonlinear regression and one site binding equation. The IC₅₀ value of the HLA-B60 binder positive control peptide KESTLHLVL (9) was 1.1 μM.

FIG. 15. Stable binding of nonameric and decameric HA-1^{H/R} peptides to HLA-B60. The nonameric and decameric HA-1^{H/R} peptides were tested for binding to HLA-B60 (a, b) and to HLA-A2 (c) at the indicated temperatures. The results are expressed as the percentage inhibition of HLA the binding of the reference peptide. Curves were fitted by nonlinear regression and one site binding equation.

FIG. 16. T cell recognition of HLA-B60/HA-1^{H} ligand. T cell lines (TCL) secreting IFN-γ in response to the target cells indicated. The EBV-LCLs (HLA-B60/HA-1^{RR} and HLA-B60/HA-1HR) are derived from HLA identical but HA-1 non-identical siblings. On the Y-axis, the number of IFN-7 spots per 10⁵ cells is expressed. The SEM was <5%.

Table 6. KIAA0223 sequence polymorphism in mH HA-1 positive and HA-1 negative individuals. Sequencing of HA-1 region in KIAA0223 gene in HA-1+/+ and HA-1−/− homozygous individuals and KG-1 revealed two alleles differing in two nucleotides resulting in a one amino acid difference (H to R) and designated HA-1^{H} and HA-1^{R}. For DH and vR 6 independent PCR products were sequenced. For KG-1 8 PCR products were sequenced.

### DETAILED DESCRIPTION OF THE INVENTION

For the sake of illustration, a number of methods and applications is also given below in the examples.

### EXAMPLES

### Example 1

GvHD is a frequent and life-threatening complication after allogeneic HLA-identical bone marrow transplantation (BMT). Recipients of HLA-identical bone marrow develop acute or chronic GvHD in respectively 36% and 49%^{1,2}. Disparities in genes other than the MHC, referred to as minor histocompatibility (mH) antigens, are clearly involved in the development of GvHD after HLA-identical BMT. A recent retrospective analysis revealed the significant association between mismatching for the mH antigen HA-1 and the induction of GvHD after HLA-identical BMT³. Minor histocompatibility antigens are recognized by MHC restricted T cells and were shown to be peptides derived from intracellular proteins presented by MHC molecules⁴⁻⁶. Here we report the first identification of a polymorphic gene encoding an human mH antigen. The GvHD associated mH antigen HA-1 is a nonapeptide derived from the di-allelic KIAA0223 gene. The HA-1 allelic counterpart encoded by the KIAA0223 gene differs only at one amino acid from the mH antigen HA-1. Family studies demonstrated an exact correlation between the KIAA0223 gene polymorphism and the HA-1 phenotype as was previously determined by recognition by the HA-1 specific CTL clones. The elucidation of the HA-1 encoding gene enables prospective HA-1 DNA typing of BMT donors and recipients to improve donor selection and prevention of GvHD.

Cytotoxic T cell clones specific for the mH antigen HA-1 have been isolated from three different patients with severe GvHD⁷. The mH antigen HA-1 is presented in the context of HLA-A2.1 and present in 69% of the HLA-A2.1 positive population⁷. HA-1 expression was demonstrated to be tissue specific and limited to cells of hematopoietic origin, including dendritic cells, Langerhans cells and leukemic cells⁸⁻¹⁰. Family analysis indicated a Mendelian mode of inheritance for HA-1 and segregation independent from the MHC complex¹¹. Comparison of the T cell receptor (TCR) sequences of different HA-1 specific T cell clones derived from different individuals revealed conserved usage of the TCR Vb6.9 and conserved amino acids in the CDR3 region¹². In a retrospective study, mismatching for a number of mH antigens was evaluated with regard to the association with GvHD after HLA-identical BMT. A single HA-1 mismatch between donor and recipient was significantly correlated with the induction of GvHD after HLA-identical BMT³.

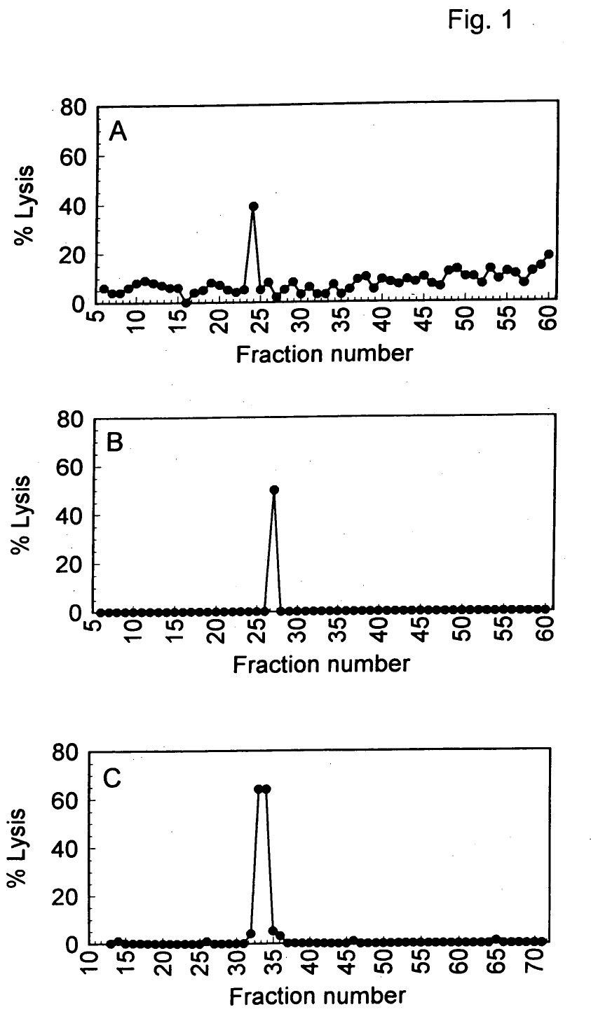

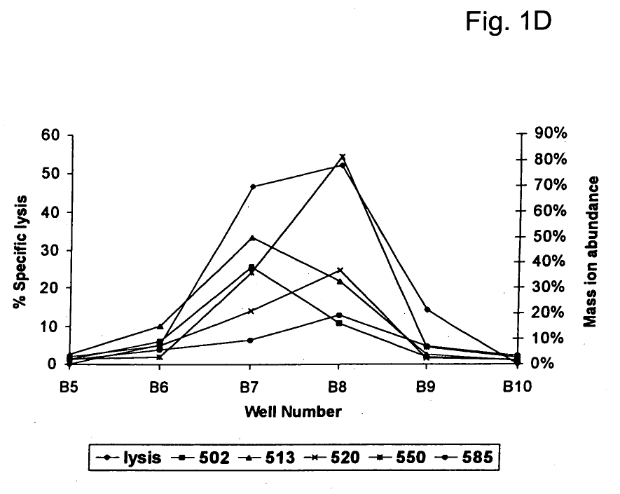

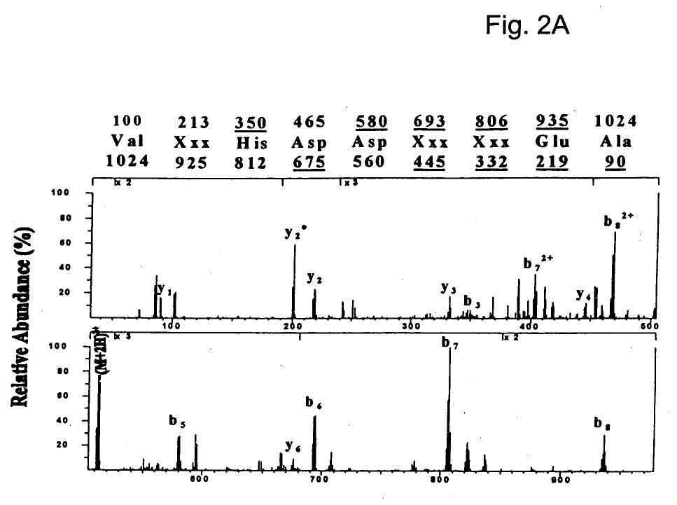

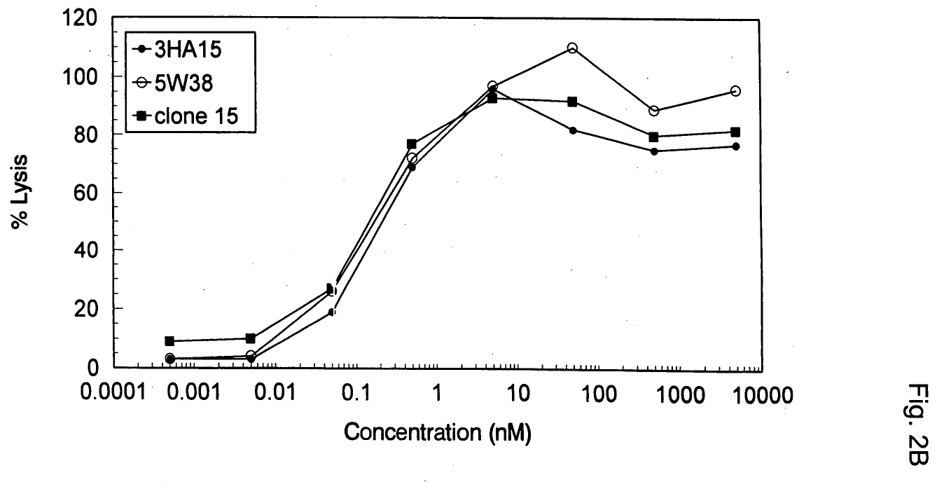

To identify the mH antigen HA-1, HLA-A2.1 molecules were purified from two HA-1 expressing EBV-transformed B lymphoblastoid cell lines (EBV-BLCL) Rp and Blk. The HLA-A2.1 bound peptides were isolated by acid treatment and fractionation of the peptides was performed by multiple rounds of reverse phase HPLC. The, fractions were analyzed for their capacity of inducing HA-1 specific lysis using T2 cells as target cells and an HA-1 specific CTL clone as effector cells in a ⁵¹Cr-release assay (FIG. 1a). Fraction 24 contained HA-1 activity and was two times further fractionated with reverse phase HPLC using a different organic modifier (FIG. 1b.c.). Fraction 33 and 34 of the third HPLC fractionation showed HA-1 activity ⁵¹Cr-release assay and were analyzed by tandem mass spectrometry. Because over a 100 different peptides were present in these fractions, around 40% of fractions 33 and 34 was chromatographed with an on-line microcapillary column effluent splitter. The fractions were simultaneously analyzed by tandem mass spectrometry and ⁵¹Cr-release assay (FIG. 1d.). Five peptide species (at m/z 550, 520, 513, 585 and 502) were specifically present in active fractions and absent in fractions without activity in the CML assay. Collision activated dissociation analysis of peptide candidate m/z 550 revealed the sequence YXTDRVMTV (SEQ ID NO:______). X stands for Isoleucine or leucine that cannot be discriminated with this type of mass spectrometer. However, a synthetic peptide with this sequence was not able to reconstitute the HA-1 epitope (results not shown). To determine which of the four remaining candidates was the HA-1 peptide the second HA-1 purification of the EBV-BLCL Blk was evaluated. HA-1 positive peptide fraction 33 of the second reverse phase HPLC fractionation was further chromatographed by microcapillary HPLC with a third organic modifier. A single peak of reconstituting activity was observed in a ⁵¹Cr-release assay (results not shown). Mass spectral analysis of these fractions revealed that only peptide candidate m/z 513 was present. This peptide was analyzed with collision activated dissociation analysis and sequenced as VXHDDXXEA (SEQ ID NO:______) (FIG. 2a). Isoleucine and leucine variants of the peptide were synthesized and run on the microcapillary HPLC column. Only peptide VLHDDLLEA (SEQ ID NO:______) coeluted with the naturally processed peptide 513 (results not shown). Next, synthetic VLHDDLLEA (SEQ ID NO:______) added in different concentration to a CML assay with 3 different HA-1 specific CTL clones revealed recognition by all three clones of the peptide with a half maximal activity at 150-200 pM for or all three clones (FIG. 2b). This demonstrated that the mH antigen HA-1 is represented by the nonapeptide VLHDDLLEA (SEQ ID NO:______).

Database searches performed to identify the gene encoding HA-1, revealed that the HA-1 peptide VLHDLLEA (SEQ ID NO:______) was identical for 8 out of 9 amino acids with the peptide VLRDDLLEA (SEQ ID NO:______) from the KIAA0223 partial complementary DNA (cDNA) sequence, derived from the acute myelogenous leukemia KG-1 cell line. Because HA-1 has a population frequency of 69%, we reasoned that VLRDDLLEA (SEQ ID NO:______) might represent the HA-1 allelic counterpart present in the remaining 31% of the population. To elaborate on this assumption, we performed cDNA sequence analysis of the putative HA-1 encoding region of KIAA0223 in EBV-BLCL derived from a presumed HA-1 homozygous positive (vR), from a presumed HA-1 negative individual (DH) and from the KG-1 cell line (Table 6.). The HA-1 encoding region of KIAA0223 of the HA-1+/+ individual (vR) displayed two nucleotides differences from the KIAA0223 sequence in the databank, leading to the amino acid sequence VLHDDLLEA (SEQ ID NO:______) (designated HA-1^{H}). The HA-1 encoding region of KIAA0223 of the HA-1−/− individual (DH) showed 100% homology with the reported KIAA0223 sequence (designated HA-1^{R}). The KG-1 cell line expressed both KIAA0223 alleles. Because KG-1 does not express the restriction molecule HLA-A2.1 necessary for T cell recognition, we transfected KG-1 with HLA-A2.1 and used these cells as target cells in a ⁵¹Cr-release assay with the HA-1 specific T cell clone as effector cells. According to the cDNA sequence analysis results, the KG-1 cells were recognized by the HA-1 specific T cell clone (data not shown). This result suggested that the KIAA0223 gene forms a di-allelic system of which the HA-1^{H} allele leads to recognition by the mH antigen HA-1 specific T cell clones.

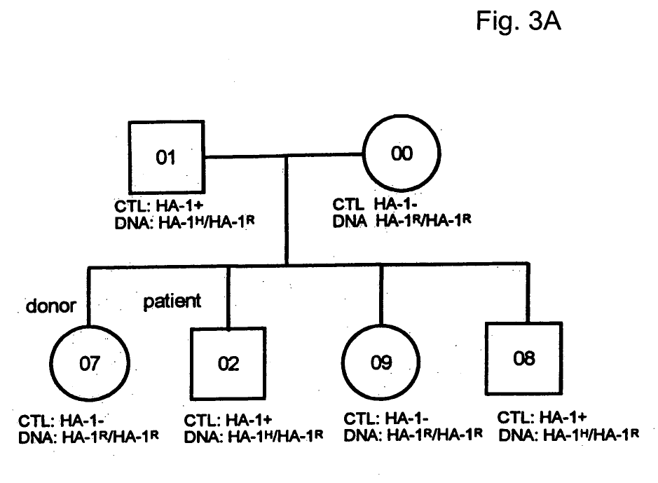

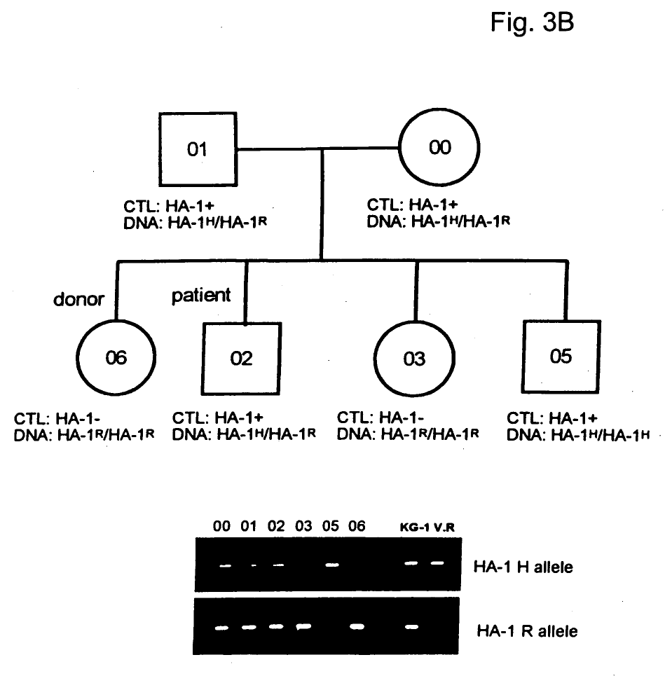

Two families, who were previously typed for HA-1 with HA-1 specific CTL were studied on the cDNA level for their K1AA0223 polymorphism. The family members of family 1 were screened for their KIAA0223 sequence polymorphism by sequencing the HA-1 encoding sequence region. All HA-1 negative members displayed the HA-1R sequence, whereas all HA-1 positive members turned out to be heterozygous, thus carrying both HA-1 alleles (FIG. 3a). We subsequently designed HA-1 allele specific PCR primers to screen another family previously cellularly typed for HA-1. Both parents and one child were determined as heterozygous for HA-1, two HA-1 negative children homozygous for the HA-1^{R} allele and one child homozygous for the HA-1^{H} allele (FIG. 3b). The screening of both families showed an exact correlation of the HA-1 phenotype as determined by recognition by the HA-1 specific T cell clones and the KIAA0223 gene polymorphism.

To definitely prove that the KIAA0223 gene encodes the mH antigen HA-1, the HA-1 encoding sequence region of KIAA0223 of both the HA-1^{H} and the HA-1^{R} alleles were cloned in a eukaryotic expression vector and transiently transfected in HA-1 negative Hela cells in combination with HLA-A2.1. HA-1 specific T cell recognition of these transfected Hela cells was assayed using a TNFa release assay. The Hela cells transfected with the HA-1^{H} sequence containing vector were recognized by two HA-1 specific T cell clones (FIG. 3c). In contrast transfection of the HA-1^{R} sequence containing vector did not lead to recognition. In conclusion, our results clearly demonstrate that the mH antigen HA-1 is encoded by the HA-1^{H} allele of the KIAA023 gene.

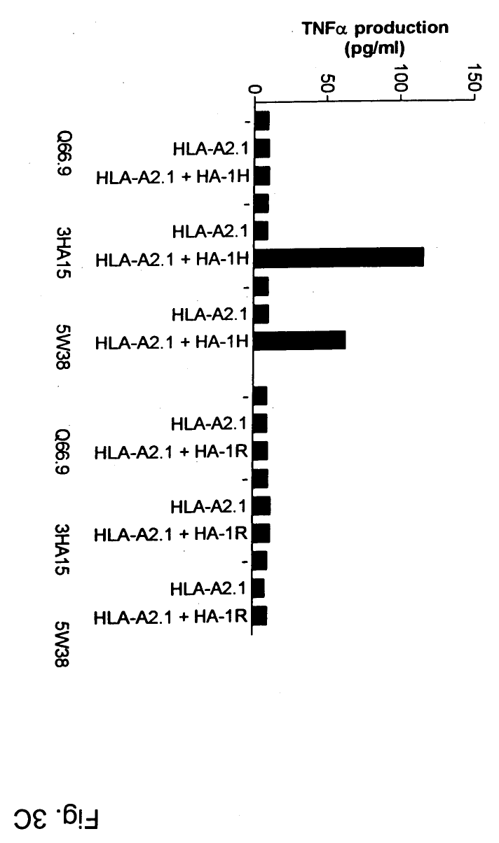

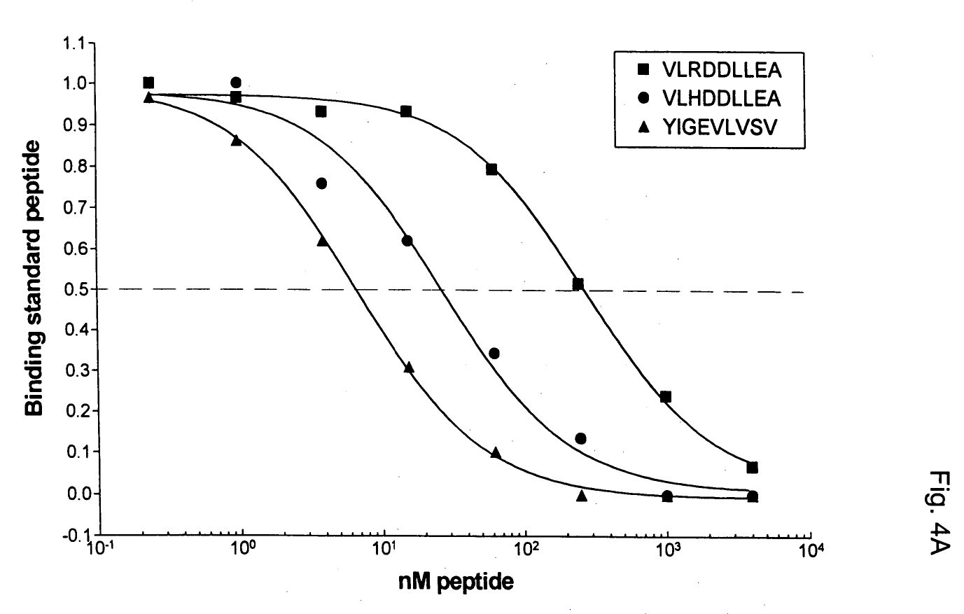

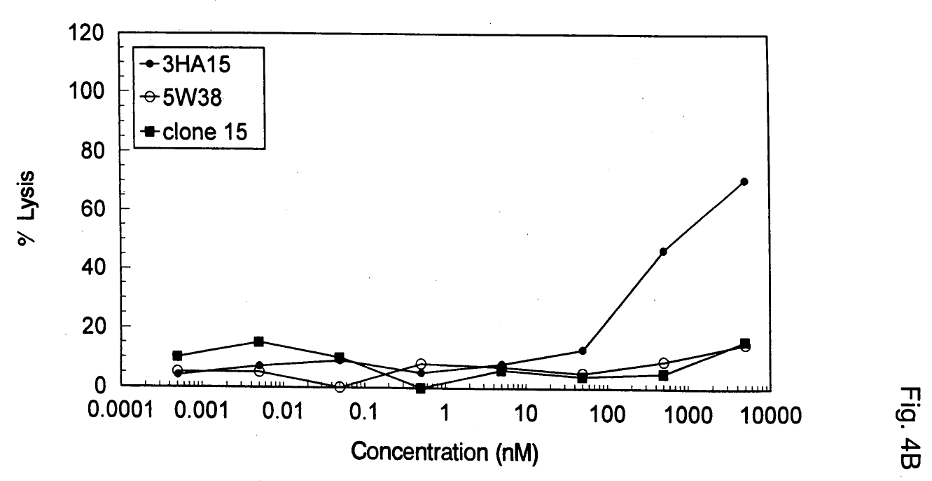

Reconstitution and HLA-A2.1 binding assays were performed to determine the capacity of HA-1^{R} peptide VLRDDLLEA (SEQ ID NO:______) to bind to HLA-A2.1 and to be recognized by the HA-1 specific T cell clones. The concentration of the HA-1^{R} peptide that inhibited the binding of a fluorescent standard peptide to HLA-A2.1 by 50% (IC50) was 365 nM, falling in the intermediate binders, whereas the IC50 of the HA-1^{H} peptide was 30 nM, which is in the range of high affinity binders (FIG. 4a)^{13,14}. Different concentrations of VLRDDLLEA (SEQ ID NO:______) were tested in a ⁵¹Cr-release assay with three HA-1 specific T cell clones. One out of the three clones (3HA15) tested showed recognition of the HA-1^{R} peptide, but only at 1000 times higher peptide concentration than that necessary for the recognition of the HA-1^{H} peptide (FIG. 4b). As the binding affinity of the two peptides to HLA-A2.1 differs only 10-fold, it can be concluded that all the T cell clones specifically recognize the HA-1^{H} peptide.

The 3HA15 T cell clone, recognizing the HA-1^{R} peptide at high concentrations, does not recognize HA-1^{R} homozygous individuals. This suggests that VLRDDLLEA (SEQ ID NO:______) is not presented by HLA-A2.1 or presented below the detection limit of the T cell. To determine whether the HA-1^{R} peptide VLRDDLLEA (SEQ ID NO:9 was presented by HLA-A2.1, HLA-A2.1 bound peptides were eluted from an HA-1^{R} homozygous EBV-BLCL and fractionated with reverse phase HPLC. The synthetic HA1-peptide VLRDDLLEA (SEQ ID NO:______) was run on reverse HPLC to determine at which fraction this peptide eluted. The corresponding HPLC fractions derived from the, HA-1^{R} expressing EBV-BLCL were analyzed using mass spectrometry. Presence of peptide VLRDDLLEA (SEQ ID NO:______) could not be detected (results not shown), indicating that this peptide is not or in very low amounts presented by HLA-A2.1 on the cell surface. This is most likely due to the 10-fold lower binding affinity of the peptide for HLA-A2.1. The supposed absence of the HA-1^{R} peptide in HLA-A2.1 indicates that this allele must be considered as a null allele with regard to T cell reactivity. This implicates that only BMT from an HA-1^{R/R} (HA-1-) donor to HA-1^{H/H} or HA-1^{R/H} (HA-1+) recipient direction and not the reverse would be significantly associated with GvHD. This is indeed observed in a retrospective study in which HLA-2.1 positive BMT pairs were typed for HA-1³. However, HA-1^{R} derived peptides may bind to other HLA alleles and possibly be recognized by T cells. If the latter peptides are not generated and presented by the HA-1^{H} allele, then T cell reactivity towards the HA-1^{R} allele may be envisaged and GvHD in that direction may occur.

Only a few murine and human mH antigens have been identified so far on the peptide and gene level. Two murine mH antigens are encoded by mitochondrial proteins, leading to respectively four and two alleles¹⁵⁻¹⁷, In addition, two murine H-Y mH antigens were shown to be peptides encoded by Y-chromosome located genes¹⁸⁻²¹. The human SMCY gene, located on the Y chromosome, encodes the HLA-B7 and the HLA-A2.1 restricted H-Y mH antigens^{5,6}. Of the human non-sex linked mH antigens only the mH antigen HA-2 has been sequenced on the peptide level, but the HA-2 encoding gene remained unknown 4. The identification of the gene encoding the mH antigen HA-1 is the first demonstration that human mH antigens are derived from polymorphic genes. The HA-1 encoding KIAA0223 gene has two alleles differing in two nucleotides leading to one single amino acid difference. However, because the KIAA0223 gene has not been fully sequenced yet, it remains to be established whether additional amino acid polymorphisms between the two alleles of this gene are present.

Because the HA-1 mH antigen is the only known human mH antigen that is correlated with the development of GvHD after BMT the results of our study are of significant clinical relevance³. Although the numbers of different human mH antigens is probably high, it is envisaged that only few immunodominant mH antigens can account for the risk for GvHD²². Identification of those human immunodominant mH antigens and screening for those antigens may result in a significant decrease in GvHD after BMT. Here we describe the first elucidation of a polymorphic gene encoding the immunodominant mH antigen HA-1. This enabled us to design HA-1 allele specific PCR primers for pre-transplant donor and recipient typing to improve donor selection and thereby prevention of HA-1 induced GvHD development.

It also enabled us to start targeting leukemic cells carrying minor antigens present on hematopoietic cells. One way of arriving at agents targeting leukemic cells, is the ex vivo preparation of CTL's. This is explained herein below.

Allogeneic bone marrow transplantation (BMT) is a common treatment of hematological malignancies²⁹. Recurrence of the underlying malignancy is a major cause of treatment failure^{30,31}. Relapsed CML patients can be successfully treated by donor lymphocyte infusions (DLI)^{32, 33}, but the treatment is less effective for relapsed AML and ALL^{32, 33}, and is frequently complicated with GvHD³²⁻³⁴. Donor derived CTLs specific for patients' minor histocompatibility antigens (mHags) play an important role in both GvHD and GvL reactivities^{10, 35-38}. mHags HA-1 and HA-2 induce HLA-A2 restricted CTLs in vivo. mHags HA-1 and HA-2 are exclusively expressed on hematopoietic cells including leukemic cells^{10, 36} and leukemic precursors^{37, 38}, but not on cells of the GvHD target organs such as skin fibroblasts, keratinocytes or liver cells⁸. Recently the chemical nature of the mHags HA-1 and HA-2 was unraveled^{4, 39}. Here we report on the feasibility of ex-vivo generation of mHag HA-1 and HA-2 specific CTLs from unprimed mHag HA-1 and/or HA-2 negative healthy blood donors with the purpose of adoptive immunotherapy of relapsed leukemia with a low risk of GvHD.

To define the optimal APC for ex vivo generation of HA-1 and HA-2 specific CTLs, we prepared peripheral blood mononuclear cells (PBMC), monocytes, peripheral blood circulating dendritic cells (PBDC) or dendritic cells derived from bone marrow CD34+ progenitor cells (BMDC) from fifteen HLA-A2 positive, HA-1 or HA-2 negative healthy blood donors. These APCs were pulsed with HA-1 and/or HA-2 synthetic peptides and used to stimulate autologous unprimed CD8⁺ T cells. The attempts to induce HA-1 or HA-2 specific CTLs using monocytes or PBMC were not very successful. PBMC induced in only one out of three attempts HA-2 specific CTLs. Using monocytes, we generated two HA-1 peptide specific CTLs, but these CTLs did not lyse HA-1 positive target cells in our experiments (data not shown). It is possible that these “peptide specific” CTLs have a lower affinity for the naturally expressed HA-1 antigen, but this does not mean that these cells can not be used for generating CTL's against minor antigens.

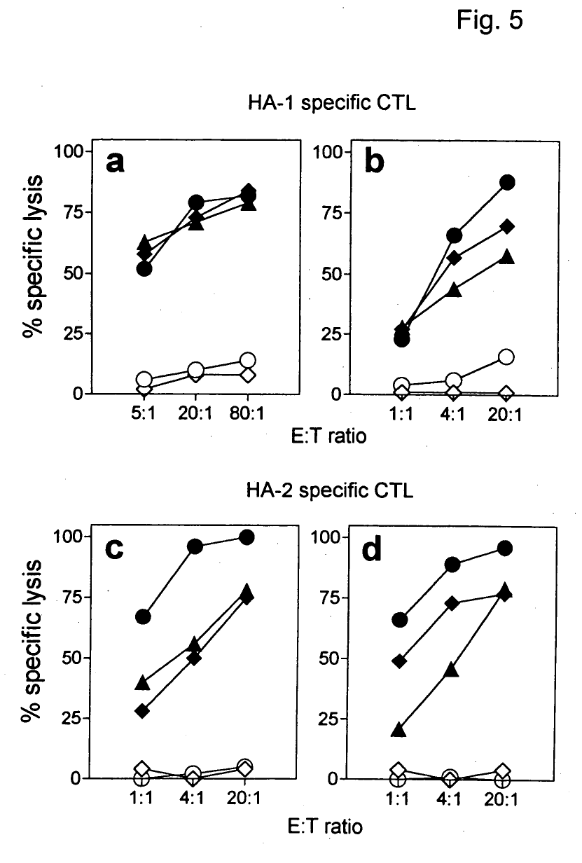

PBDC were enriched from nine individuals to induce HA-1 or HA-2 specific CTLs. In the four cases where the preparations had a purity of less than 30% the CTLs lysed peptide loaded target cells but not mHag positive target cells (data not shown). In contrast, in all cases (n=5) where PBDC purity was 30% or more, the CTLs not only recognized mHag negative, peptide pulsed target cells, but also mHag positive EBV-LCL, demonstrating the recognition of the naturally expressed ligand (FIG. 1). These results underscore the superior capacity of DC to induce T cell responses from naive precursors and confirm the current opinion⁴⁰. Similarly, two BMDC induced CTLs that recognized both peptide pulsed target cells and HA-1 positive target cells (FIG. 5). No cytotoxic activity was observed against autologous PHA stimulated T cell blasts (PHA blasts) or against mHag negative EBV-LCL. Thus, neither autoreactivity nor “third-party” alloantigen reactivity was observed. Several HA-1 or HA-2 specific CTL clones isolated from these CTLs did not react against autologous cells either. These results show that HA-1 and HA-2 specific CTLs can be safely transferred to patients after BMT.

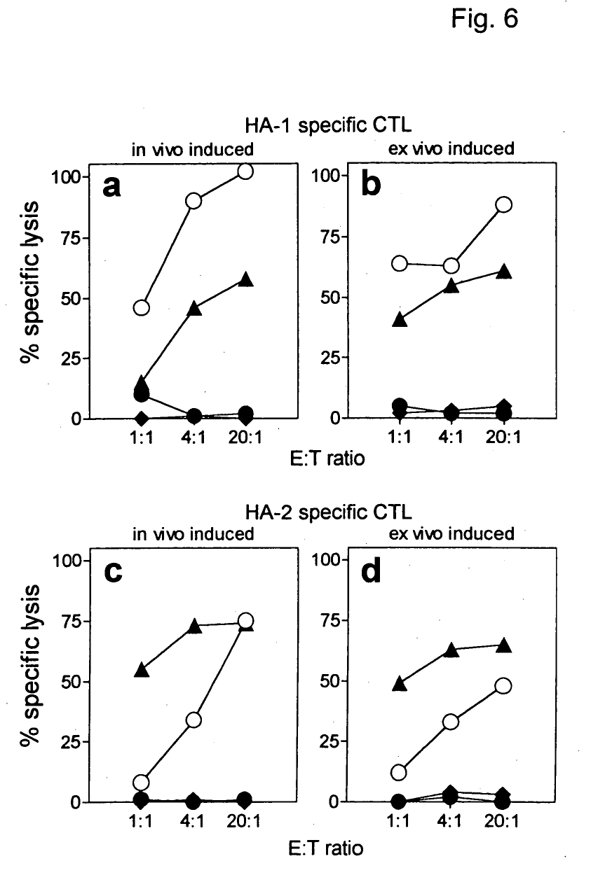

The ex-vivo induced HA-1 and HA-2 specific CTLs were tested for their hematopoietic cell restricted reactivity and compared with the in vivo induced HA-1 and HA-2 specific CTLs (FIG. 6). PHA blasts, but not fibroblasts (neither after IFN-g/TNF-a stimulation) were recognized by both ex-vivo and in vivo induced HA-1 and HA-2 specific CTLs. Fibroblasts, were only lysed after pulsing with the mHag peptides, demonstrating their susceptibility to CTL mediated lysis. These data not only confirm that the HA-1 and HA-2 antigens are functionally expressed solely on hematopoietic cells⁸, but also show that adoptive transfer of HA-1 or HA-2 specific CTLs to HA-1 or HA-2 positive patients will spare the patient's non-hematopoietic tissues and cells. Thus, upon adoptive transfer of HA-1 and HA-2 specific CTLs, a low risk of GvHD is to be expected. Some precaution may be necessary since we have previously demonstrated that HA-1 disparity between patient and donor is associated with the development of GvHD in adults³. Therefore we transfer the CTLs not before 50-60 days post BMT. It is assumed that most recipient hematopoietic cells are then be replaced by donor cells. Alternatively, one may transduce the HA-1 and HA-2 specific CTLs with a suicide gene which will make the in vivo elimination of cells possible if adverse effects occur⁴¹.

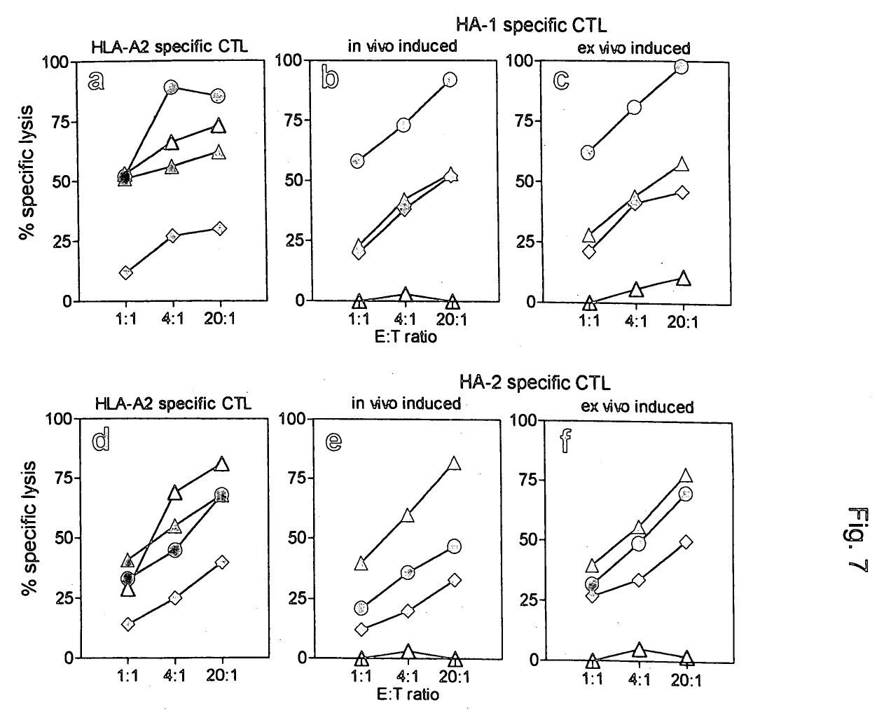

The ex-vivo induced HA-1 and HA-2 specific CTLs were subsequently analyzed for cytolytic activity against, for this study most relevant target cells, leukemic cells. In vivo induced HA-1 and HA-2 specific CTLs and an HLA-A2 specific alloreactive CTL were used as control effector cells. As shown in FIG. 7, AML and ALL cells were lysed by HLA-A2 specific alloreactive CTL, and by in vivo induced HA-1 and HA-2 specific CTLs, indicating that the leukemic cells were positive for HLA-A2 and expressed HA-1 or HA-2 antigens. As expected the ex-vivo induced CTLs lysed the leukemic cells comparable to the control effector cells. These results show that HA-1 and HA-2 specific CTLs can also be used as therapy for relapsed AML or ALL, which are resistant to DLI treatment. The level of cytotoxicity could be significantly enhanced following IFN-g and TNF-a treatment of the leukemic cells indicating that cytokines upregulated HLA class I expression on the leukemic cells. HA-1 and HA-2 specific CTL clones produce IFN-g and TNF-a ex vivo. It is possible that cytokine production by HA-1 and HA-2 specific CTLs occurs in vivo as well. Alternatively the efficacy of adoptive immunotherapy with HA-1 and HA-2 specific CTLs may be enhanced by co-administration of IFN-a in resistant cases.

The feasibility of adoptive immunotherapy with ex-vivo generated CTLs depends also on their expandability to sufficient numbers. We therefore scored the expansion rates of HA-1 and HA-2 specific CTLs generated by DC. The results indicate that sufficient numbers of CTLs for adoptive immunotherapy can be obtained if T cell cultures will be started with 5×10⁷ responder cells. For instance two HA-2 specific CTLs induced by PBDC showed expansion rates of above 9, 25 and 8 fold at the second, third, and fourth week, respectively. These expansion rates translate into an estimated total yield of 3×10⁹-10¹⁰ CTLs at the end of the fourth week. The expansion kinetics of the HA-1 specific CTLs were slower, but the cells expanded consistently with doubling times of 2-3 days during each restimulation. It is estimated that 10⁹ HA-1 specific CTLs can be obtained after five weeks of culture.

In conclusion, our results show for the first time that mHag HA-1 and HA-2 specific CTLs can reproducibly be generated ex-vivo from HLA-A2 positive, mHag HA-1 and/or HA-2 negative healthy blood donors using dendritic cells pulsed with synthetic peptides. After the successful application of EBV-specific CTLs as specific adoptive immunotherapy of EBV-related malignancies⁴², our results now provide a new possibility for the treatment of relapsed, HA-1 and/or HA-2 positive leukemia patients with HA-1 or HA-2 specific CTLs induced ex-vivo from their HLA identical, mHag negative bone marrow donors.

Methods

Cell culture. The CD8+ HLA-A2.1 restricted HA-1 specific cytotoxic T cell clones 3HA15, clone 15 and 5W38 were derived from PBMC of two patients who had undergone HLA identical bone marrow transplantation^{7,23}. The clones were cultured by weekly stimulation with irradiated allogeneic PBMC and BLCL in RPMI-1640 medium containing 15% human serum, 3 mM 1-glutamine, 1% Leucoagglutinin-A and 20U/ml rIL-2. The HLA-A2.1 positive HA-1 expressing EBV transformed B cell lines (BLCL) Rp and Blk were maintained in IMDM containing 5% FCS. The KG-1 and T2 cell lines were cultured in 1640 medium containing 3 mM 1-glutamine and 10% FCS.

⁵¹Cr-release assay. HPLC fractions and synthetic peptides were tested in a ⁵¹Cr-release assay as described²⁴. 2500 ⁵¹Cr labeled T2 cells in 25 ml were incubated with 25 ml peptide dissolved in Hanks 50 mM Hepes for 30 minutes at 37° C. Cytotoxic T cells were added in an end volume of 150 ml. When HPLC peptide fractions were tested, T2 was incubated with 2 mg/ml MA2.1 during the ⁵¹Cr labeling. After 4 hours at 37° C. the supernatants were harvested.

Peptide purification. Peptides were eluted out of purified HLA-A2.1 molecules as earlier described²⁴. Briefly, HLA-A2.1 molecules were purified two times from 90.10⁹ HLA-A2.1 positive EBV-BLCL by affinity chromatography with BB7.2 coupled CNBR-activated sepharose 4B beads (Pharmacia LKB) and extensively washed. Peptides were eluted from the HLA-A2.1 with treatment with 10% acetic acid, further acidified by 1% TFA and separated from the HLA-A2.1 heavy chain and b2-microglobulin by filtration over a 10 kD Centricon (Amicon) filter. Peptides were fractionated using reverse phase micro HPLC (Smart System, Pharmacia). For the first purification three rounds of HPLC fractionation were used to purify the HLA-A2.1 restricted HA-1 active peptide fractions from 90.10⁹ Rp cells. The first fractionation consisted of buffer A: 0.1% HFBA in H2O, buffer B: 0.1% HFBA in acetonitrile. The gradient was 100% buffer A (0 to 20 min), 0 to 15% buffer B (20 to 25 min) and 15 to 70% buffer B (25 to 80 min) at a flow rate of 100 ml/min. Fractions of 100 ml were collected. Fraction 24 of the first gradient was further fractionated. The second fractionation consisted of buffer A: 0.1% TFA in H2O, buffer B: 0.1% TFA in acetonitrile. The gradient was 100% buffer A (0 to 20 min), 0 to 12% buffer B (20 to 25 min), and 12 to 50% buffer B (25 to 80 min) at a flow rate of 100 ml/min. Fractions of 100 ml were collected. A shallower third gradient was used to further purify fraction 27 that contained HA-1 activity. The gradient was 100% buffer A (0 to 29 min), 0 to 18% buffer B (29 to 34 min), 18% buffer B (34 to 39 min), 18 to 23.9% buffer B (39 to 98 min) at a flow rate of 100 ml/min. 1/180 to 1/45 of the starting material was used to test for positive fractions in the ⁵¹Cr-release assay. Comparable HPLC fractionations were used for the second purification of HLA-A2.1 restricted HA-1 active peptide fractions from 90.109 Blk. 40% of the HA-1 containing fraction 33 of the second HA-1 purification was used for an additional reverse phase microcapillary HPLC fractionation. Buffer A was 0.1% triethyl amine (TEA) in water buffered to pH 6.0 with acetic acid and buffer B was 0.085% TEA in 60% acetonitrile buffered to pH 6.0 with acetic acid. The gradient was 100% buffer A (0 to 5 min), 0 to 100% B (5 to 45 min) at a flow rate of 0.5 ml/min. Fractions were collected in 50 ml of 0.1% acetic acid every minute for 5 to 15 minutes, every 30 seconds from 15 to 20 minutes, every 20 seconds from 20 to 40 minutes, and every 30 seconds from 40 to 45 minutes. For each fraction collected, 20% was used to test for HA-1 activity and 80% was used to obtain mass spectral data.

Mass spectrometry. Fractions from third dimension HPLC separation of the Rp purification that contained the HA-1 activity were analyzed by microcapillary HPLC-electrospray ionization mass spectrometry 25. Peptides were loaded onto a C18 microcapillary column (75 mm i.d.×10 cm) and eluted with a 34 minute gradient of 0 to 60% B, where solvent A was 0.1 M acetic acid in water and solvent B was acetonitrile at a flow-rate of 0.5 ml/min. One-fifth of the effluent was deposited into the wells of a 96-well plate containing 100 ml of culture media in each well (10 seconds fractions), while the remaining four-fifths was directed into the electrospray source of the TSQ-70U. Mass spectra and CAD mass spectra were recorded on a Finnegan-MAT TSQ-7000 (San Jose, Calif.) triple quadruple mass spectrometer equipped with an electrospray ion source.

HLA-A2.1 peptide binding assay. A quantitative assay for HLA-A2.1 binding peptides based on the inhibition of binding of the fluorescent labeled standard peptide Hbc 18-27 F to C6 (FLPSDCFPSV) to recombinant HLA-A2.1 protein and b2-microglobulin was used^{26,27}. In short, HLA-A2.1 concentrations yielding approximately 40-60% bound fluorescent standard peptide were used with 15 pmol/well (150 nM) b2-microglobulin (Sigma). Various doses of the test peptides were coincubated with 100 fmol/well (1 nM) fluorescent standard peptide, HLA-A2.1 and b2-microglobulin for 1 day at room temperature in the dark in a volume of 100 ml in assay buffer. The percent of MHC-bound fluorescence was determined by gel filtration and the 50% inhibitory dose was deduced for each peptide using one-site competition non-linear regression analysis with the prism graph software. Synthetic peptides were manufactured on a Abimed 422 multiple peptide synthesizer (Abimed, Langenfeld, Germany) and were more than 90% pure as checked by reverse phase HPLC.

RT-PCR amplification and sequencing of KIAA0223 region coding for HA-1. Total or mRNA was prepared from BLCL using the RNAzol method (Cinaa/Biotecx Laboratories, Houston, Tex.) or according to manufacturer's instructions (QuickPrep mRNA purification Kit, Pharmacia Biotech). cDNA was synthesized with 1 mg RNA as template and with KIAA0223 based reverse primer 5′-GCTCCTGCATGACGCTCTGTCTGCA-3′ (SEQ ID NO:______). To amplify the HA-1 region of KIAA0223 the following primers were used: Forward primer 5′-GACGTCGTCGAGGACATCTCCCAT-3′ (SEQ ID NO:______) and reverse primer 5′-GAAGGCCACAGCAATCGTCTCCAGG-3′ (SEQ ID NO:______). Cycle parameters used were denaturation 95° C., 1 min, annealing 58° C., 1 min and extension 72° C., 1 min (25 cycles). The PCR-products were purified using the Magic PCR-Preps DNA purification System (Promega) and direct cloned using the pMosBlue T-vector kit (Amersham LIFE SCIENCE). Six independent colonies from each individual were sequenced using the T7-sequencing kit (Pharmacia Biotech).

HA-1 allele specific PCR amplification. In the case of HA-1 allele specific PCR amplification, cDNA was synthesized as described above. A PCR amplification was performed with allele specific forward primers: for the HA-1^{H} allele primer H1: 5′-CCTTGA-GAA-ACT-TAA-GGA-GTG-TGT-GCT-GCA-3′ (SEQ ID NO:______), for the HA-1^{R} allele primer R1: 5′-CCT-TGA-GAA-ACT-TAA-GGA-GTG-TGT-GTT-GCG-3′ (SEQ ID NO:______) and for both reaction the reverse primer as described above was used. Cycle parameters used were denaturation 95° C., 1 min, annealing 67° C., 1 min and extension 72° C., 1 min (25 cycles).

Cloning and expression of HA-1^{H} and HA-1^{R} allelic region of KIAA0223. A forward KIAA00223 based PCR primer containing an ATG start codon (5′-CCG-GCATGG-ACG-TCG-TCG-AGG-ACA-TCT-CCC-ATC-3′ (SEQ ID NO:______) and a reverse KIAA0223 based PCR primer containing a translational stop signal (5′-CTA-CTT-CAGGCC-ACA-GCA-ATC-GTC-TCC-AGG-3′ (SEQ ID NO:______) were designed and used in a RT-PCR reaction with cDNA derived from an homozygous HA-1^{H} and a homozygous HA-1^{R} BLCL. Cycle parameters used were denaturation 95° C., 1 min, annealing 60° C., 1 min and extension 72° C., 1 min (25 cycles). The desired PCR-products were purified using the Magic PCR-Preps DNA purification System (Promega). The purified DNA was direct cloned using the pMosBlue T-vector kit (Amersham LIFE SCIENCE) and recloned in the eukaryotic pCDNA3.1 (+) vector under the control of a CMV promoter. Transient cotransfections were performed with HLA-A2.1 in Hela cells using DEAE-Dextran coprecipitation. After 3 days of culture HA-1 specific T cells were added and after 24 hours the TNFa release was measured in the supernatant using WEHI cells²⁸.

Peptides: HA-1 and HA-2 peptides were synthesized using a semi automatic multiple peptide synthesizer^{4, 39}. The purity of the peptides was checked by reversed phase high pressure liquid chromatography (HPLC).

APCs:

PBMC were isolated by ficoll-hypaque density gradient separation of blood collected with manual hemapheresis.

Monocytes were isolated by plastic adherence during PBDC enrichment.

PBDC were enriched from PBMC by depletion of T cells, monocytes, B and NK cells as described earlier. Briefly, T cells were depleted by sheep red blood erythrocyte rosetting. Non-T cells were cultured 36 h at 37° C. in RPMI+10% autologous plasma. After depleting monocytes non adherent cells were layered on 14.5% metrizamide gradients and centrifuged. The light density PBDC were recovered from the interphase. PBDC were identified by FACS being negative for CD3, CD14, CD16 and CD19 and positive for HLA-DR. The preparations contained 2-6×10⁶ cells with a DC content of 20-50%. In some cases the light density cells were further depleted from CD14 and CD19 cells using antibody coated magnetic beads.

BMDC were differentiated from bone marrow CD34+ cells (isolated using CD34+ isolation kit, MACS, Bergisch Gladbach, Germany) by culturing with 100 ng/ml FLT3-ligand (Genzyme, Leuven; Belgium), 30 ng/ml IL-3, 25 ng/ml SCF (Genzyme) 50 U/ml TNF-a (Genzyme), 250 U/ml GM-CSF (Genzyme) for 10 to 14 days. The cultures contained 20-60% DC as detected by high levels of DR and negative expression of CD3/CD14/CD16/CD19.

Ex vivo induction of HA-1 and HA-2 specific CTLs: APC were pulsed with HA-1 or HA-2 peptides (both 10 mg/ml) for 90 min. at 37° C. in serum free AIM-V medium. After washing, APC and 10-15×10⁶ responder cells (CD4 depleted autologous PBMC) were cultured at different APC: responder cell ratios depending on the type of APC (5:1, 1:3 and 1:10 for PBMC, Mo and DC, respectively) in 24 well culture plates. Culture medium was RPMI supplemented with 10% autologous plasma, 1 U/ml IL-2 (Cetus), 1 U/ml IL-12 (Genzyme). The cells were kept at 37° C. in an humidified, 5% CO₂₋₋air mixture. At day 5, 10 U/ml of IL-2 was added. Starting from day seven, the T cell cultures were restimulated weekly with peptide pulsed autologous monocytes. 10 U/ml of IL-2 was added 24 h. after each restimulation. The T cell lines were expanded with 10-20 U/ml IL-2 containing culture medium.

Cytotoxicity (⁵¹Cr release) assays: Standard 4 h ⁵¹Cr release assays using PHA-blasts, EBV-BLCL and fibroblasts and leukemic cells as target cells were performed as described before⁸. The percent specific lysis was calculated using the following formula: 100×(cpm experimental release−cpm spontaneous release)/(cpm maximal release−cpm spontaneous release).

Target cells: EBV-BLCL were generated as described before⁸ and cultured in RPMI plus 10% FCS. PHA activated T cell blasts (PHA-blasts) were obtained by stimulation of PBMC with 0.1 mg/ml PHA (Wellcome) during 72 h. PHA-blasts were expanded with medium containing 20 U/ml IL-2. Skin fibroblasts of an HLA-A2+, HA-1+, HA-2+ healthy individual were isolated, cultured and tested as described before⁸. In short, fibroblasts were trypsinized and cultured in the wells of 96 well flat bottomed microtiter culture plates at a concentration of 3×10³ cells/well with or without addition of IFN-g and TNF-a (both 300 U/ml) during 72 h. When indicated, target cells were pulsed with HA-1 or HA-2 peptides (both 10 mg/ml) during ⁵¹Cr labeling.

Leukemia patients' (AML or ALL) PBMC or BM containing >95% morphologically recognizable malignant cells were assigned as leukemic cells. Leukemic cells were thawed and cultured in RPMI plus 10% human serum for 72 h with or without addition of IFN-g and TNF-a (both 300 u/ml) before using as target cells.

In vivo induced mHag specific T cell clones: In vivo induced, mHag HA-1 and HA-2 specific CD8+ CTL clones were isolated from post BMT leukemia patients, and were documented in detail³⁵.

### Example 2

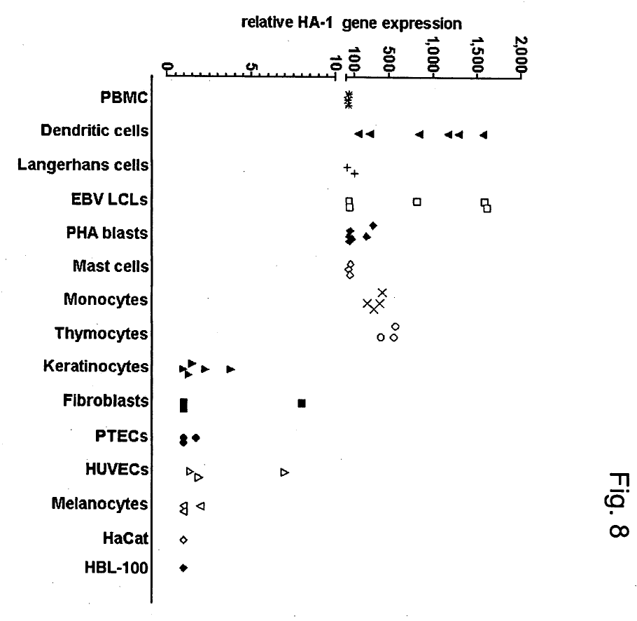

To confirm the hematopoietic system restricted tissue distribution, earlier analyzed by HA-1 specific CTLs, HA-1 mRNA levels were analyzed by quantitative real-time PCR (Example 4) in eight different hematopoietic and six different non-hematopoietic cell types. Only cells of hematopoietic origin expressed significant levels of the HA-1 gene (FIG. 8). No significant HA-1 gene expression was detected in cells of non-hematopoietic origin: i.e., keratinocytes, dermal fibroblasts, proximal tubular epithelial cells (PTECs), human umbilical vein endothelial cells (HUVECs), melanocytes and SV 40 immortalized breast cell lines HaCaT and HBL 100 (FIG. 8).

Next, we investigated the HA-1 gene transcription levels in 35 epithelial tumor cell lines derived from different carcinomas (Table 1). The HA-1 gene transcription, analyzed by quantitative real time RT-PCR, revealed significant HA-1 mRNA in twenty-six out of the thirty-five cell lines of various malignant origins. Table 1 also lists the results of the common leukocyte antigen CD45. We compared the HA-1 and CD45 RNA expression in various hematopoietic cells. Both genes are expressed in hematopoietic cells to comparable levels (data not shown). None of the tumor cell lines showed significant CD45 gene expression. This shows that HA-1 transcription observed in the tumor cell lines is specific and not due to contaminating HA-1 positive hematopoietic cells (Table 1).

Functional recognition by HA-1 specific CTLs is a prerequisite for tumor specific targeting in immunotherapeutic settings. The mHag HA-1 locus encodes two alleles i.e., the HA-1H and the HA-1R allele. The HA-1^{H} allele is the T cell epitope that is recognized by the HLA-A2 restricted CTL (52). Therefore, we executed CTL recognition studies (Example 5) on the tumor cell lines that expressed both the HLA-A2 restriction molecule and the HA-1H T cell epitope required for the HLA-A2 restricted HA-1 specific CTL recognition. Hereto, all tumor cell lines listed in table 1 were HLA and HA-1 genotyped (52). Table 2 shows significant HA-1 CTL lysis on four of the five cell lines by two HA-1 specific clones which could be enhanced in all cases by IFNγ and TNFα treatment of the target cells. The colon carcinoma cell line CaCo-2 was only recognized by one of the two HA-1 CTL clones.

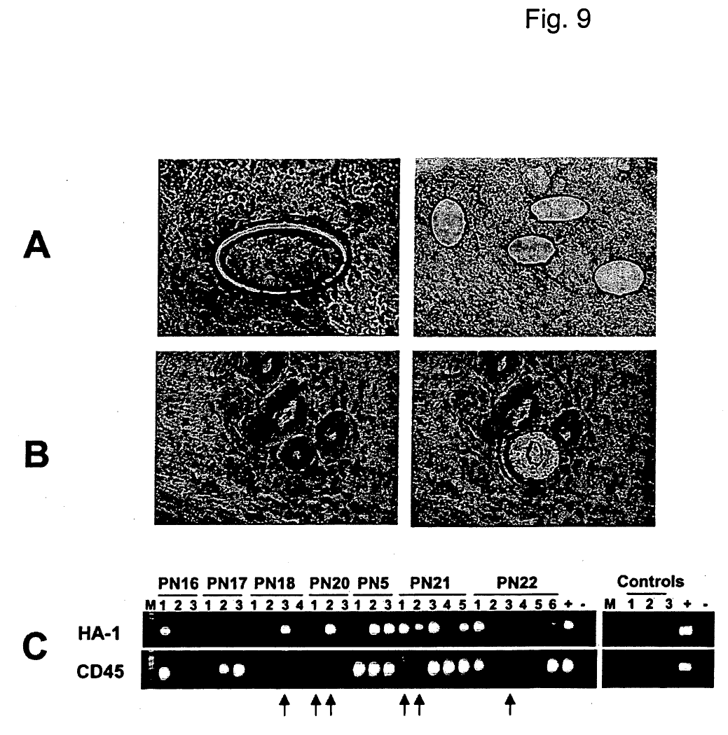

With the demonstrated functional expression of HA-1 by epithelial tumor cell lines, we expected that HA-1 is also expressed by epithelial tumors in vivo. However, given the expression of HA-1 by cells of the hematopoietic lineage and in view of the virtual omnipresence of hematopoetic cells in tumors, spurious positive results of a PCR analysis caused by contaminating hematopoietic cells should be avoided. To this end, we applied laser-mediated micro-dissection to cryosections of fresh frozen cancer samples without any microscopically visible leukocyte infiltration (Example 6, FIG. 9A). As control, we used micro-dissected normal breast glands from three patients that underwent breast reduction surgery (FIG. 9B). By the applied micro-dissection method the selected area is cut by a laser beam and directly catapulted into the reaction tube, practically excluding contamination by surrounding tissue. Of twelve tumors obtained from patients with breast and lung cancers and the three biopsies from normal breast tissue, areas of 10,000-60,000 μm² in total (comprising about 30-200 cells) were laser-micro-dissected. mRNA was isolated, reverse transcribed and amplified with a recently developed global amplification method (Example 7). Successful global amplification of cDNA was checked by established gene specific amplification of the two housekeeping genes b-actin and EF-1a and cDNA array hybridization (not shown). Following dilution of the primary PCR products, specific primers served to detect HA-1 gene expression (Example 8).

While seven of twelve tumors were positive for HA-1, all normal breast glands were negative (FIG. 9C). The identity of the PCR bands as HA-1 was confirmed by Southern blotting (not shown). We used CD45 gene specific PCR to test whether HA-1 expression might be attributed to single infiltrating leukocytes or intravascular cells that had escaped our attention. Absence of CD45 mRNA would provide strong evidence that the HA-1 signal originates from the epithelial tumor cells in vivo. Indeed, four of seven tumor samples solely expressed HA-1 (FIG. 9C, arrows) in at least one of the micro-dissected areas, whereas three tumors co-expressed CD45 and HA-1 prohibiting evaluation of their HA-1 status. Therefore, HA-1 was found to be expressed in at least 30% human primary tumors of epithelial origin in vivo.

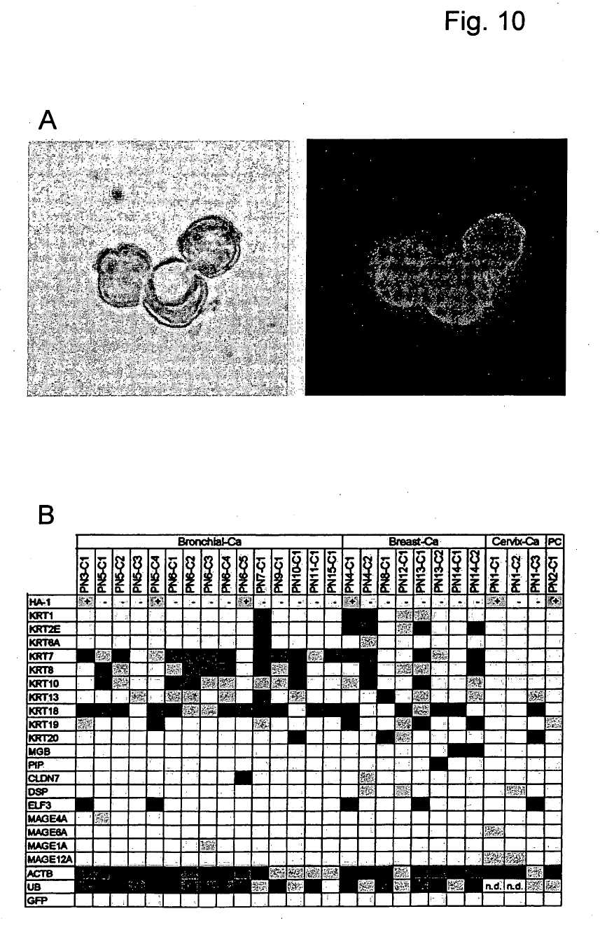

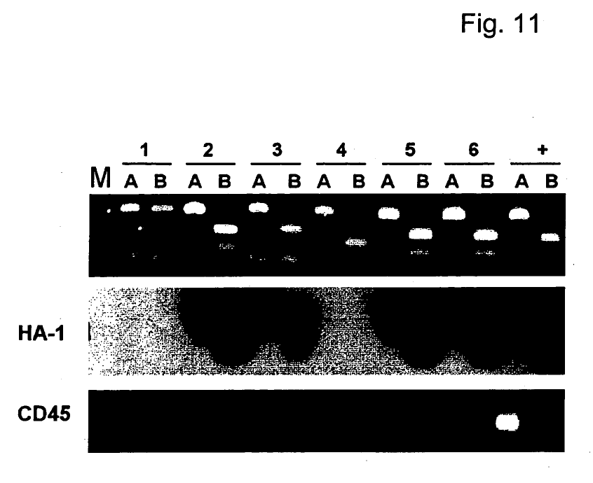

Since contamination by CD45 positive non-epithelial cells could not be absolutely excluded as cause of the encountered CD45 expression in some of the micro-dissected tumor areas, we resorted to HA-1 analysis of single tumor cells or defined cell clusters freshly isolated from bone marrow or lymph nodes of cancer patients (FIG. 10A). Single disseminated cancer cells were detected in cell suspensions prepared from bone marrow and lymph node samples with a fluorescent labeled monoclonal antibody against the epithelial cell adhesion molecule (EpCAM) as marker (53). In total, twenty-seven single tumor cells or small cell clusters were isolated by micromanipulation from fifteen cancer patients (FIG. 10A). For cDNA analysis, the same global amplification technique was applied that was used for the micro-dissected tumor areas, enabling faithful detection of expressed transcripts in single cells (Example 7). The labeled cDNAs were hybridized to an array including specific epithelial marker genes such as the cytokeratin family members (KRT), mammaglobin (MBG) and prolactin induced protein (PIP) as markers for breast-derived cells, and the transcription factor ELF3. Further evidence of epithelial origin was provided by claudin 7 (CLDN7) and desmoplakin I (DSP) both involved in epithelial cell adhesion. As indicator of malignancy, the expression of MAGE genes was analyzed, the transcripts are found in spermatogonal cells and exclusively in various cancer cells, hence the designation cancer-testis genes. In addition, we evaluated the cells for markers of hematopoietic cells such as the T cell receptor, CD45, CD33, CD34, CD37, CD38, and CD16. The isolated cells expressed none of the hematopoietic markers (not shown). Expression of cytokeratins and other epithelial markers indicated their epithelial origin (FIG. 10B). In some cases the cells were positive for one or more MAGE genes suggesting their tumor origin, despite down-regulation of cytokeratin mRNA (FIG. 10B). All cells were then tested for HA-1 and CD45 expression by gene specific PCR; the HA-1 amplification products were subsequently confirmed by restriction enzyme digest and by Southern blotting (Example 8). Six of the twenty-seven cells expressed the HA-1 gene and none of them expressed the CD45 gene (FIG. 11). The HA-1 significant transcripts were observed in samples derived from breast cancer (PN4—C1), bronchial carcinoma (PN3-C1, PN5-C1, PN6-C5), prostate cancer (PN2-C1) and cervical cancer (PN1-C1). From two of the HA-1 positive cells (PN5-C4, PN3-C1) we could besides mRNA also evaluate their DNA by a recently described method (C. A. Klein submitted and 53). The isolated DNA was subjected to whole genome amplification and comparative genomic hybridization (CGH). Both cells harbored multiple genomic alterations, lending ultimate proof of their malignant nature (FIG. 12).

### Example 3

We have investigated whether the HA-1^{H/R} polymorphic region contains peptides that can be presented by other HLA molecules than HLA-A2. Hereto, we analyzed the binding capacities of HA-1 polymorphic peptides to nine HLA-A and -B molecules that have a frequency of more than 10% in the Caucasian population. Nonameric HA-1^{H/R} peptides (n=18) were tested for binding to these frequent HLA alleles. The peptide binding analyses were extended with two decameric HA-1^{H/R} peptides that contained binding motives for HLA-A3 and with five nonameric/decameric peptides that were predicted to bind to HLA-B 14 or to -B60. Next to the binding studies, cellular processing was executed by in vitro proteasome digestion of 29 amino acid long HA-1^{H} and HA-1^{R} peptides. To enlarge the patient population for HA-1 specific immunotherapy, the HLA-B60 binding peptides were analyzed for their in vitro immunizing potential. Hereto, peptide loaded dendritic cells (DCs) were used to induce T cell responses from healthy individuals.

Materials & Methods

HA-1 Peptides:

HA-1^{H} and HA-1^{R} peptides were synthesized using an automated multiple peptide synthesizer (Syro II, Multisyntech, Witten, Germany) according to the known HA-1 amino acid sequence (61). The purity of the peptides was >90%. The peptides were dissolved in dimethyl sulfoxide (DMSO), diluted in 0.9% NaCl and stored at −20° C. until use.

Prediction of HLA Peptide Binding:

The polymorphic HA-1^{H} and HA-1^{R} regions were screened with the HLA-peptide binding prediction software of BIMAS (Bioinformatics & Molecular Analysis Section, NIH, Bethesda, Md.; url: http://bimas.dcrt.nih.gov./) for octameric, nonameric or decameric HA-1 peptides capable to bind to HLA class I molecules. The selection of peptide candidates was made by comparison of the computed scores with that of the HLA-A2 restricted HA-1^{H} CTL epitope with amino acid (aa) sequence VLHDDLLEA (SEQ ID NO:______) (score=79.6). This score corresponds to the estimated half-time of dissociation of complexes containing the peptide at 37° C. at pH 6.5. Five HA-1^{H/R} peptides with scores ranging from 32 (intermediate binding score) to 176 (strong binding score) were selected to assay for binding to the relevant HLA class I molecules. The predicted HLA class I/HA-1^{H/R} peptide associations and their computed binding scores are presented in table 3. In addition, we selected two decameric HA-1^{H/R} peptides that contained anchor residues for binding to HLA-A3 but were not predicted by the BIMAS software.

HLA Peptide Binding Assays.

We used the competition-based HLA peptide binding assay as described previously, with some modifications (67). Briefly, HLA typed EBV-LCLs were washed with PBS, kept on ice for 5 min. and treated with an ice-cold 0.132 M citric acid, 0.062 M Na₂HPO₄.2H₂O elution buffer for 90 sec (67). The pH of the elution buffer was optimized for each HLA molecule to enable maximal elution of HLA bound peptides (manuscript in preparation).

Immediately after mild acidic treatment, the cells were washed with 12 ml Iscove's modified Dulbecco's medium (IMDM, Bio Whittaker, Belgium) containing 2% FCS and resuspended in IMDM containing 2% FCS, 1.5 μg/ml O₂ microglobulin (Sigma, St. Louis, Mo., USA). 4×10⁴ acid treated EBV-LCLs were then incubated in 96-well-V-bottom plates (Costar, Cambridge, Mass., USA) with fluorescent-labeled reference peptide (25 μl/well, final concentration: 150 nM) mixed with serial dilutions of competitor (test) peptides (25 μl/well; final concentrations: 100 to 0.78 μM) in a total volume of 150 ml. All reference peptides were deduced from previously reported peptides that show strong binding to the respective HLA class I molecules (68). After incubation for 24 h at 4° C., the cells were washed twice with 100 μL/well PBS/1% FCS and fixed with 0.5% paraformaldehyde in PBS. The mean fluorescence expressed by the cells was determined by a FACScalibur flow cytometer (Becton-Dickinson, St. Louis, Mo., USA). Percentage inhibition of the HLA binding of the fluorescent reference peptide is calculated with the formula: % inhibition=1−[(mean fluorescence in the presence of competitor peptide−mean background fluorescence)/(mean fluorescence in the absence of competitor peptide−mean background fluorescence.)]×100%. The relative binding affinity of the peptides is expressed as the peptide concentration that inhibits 50% of the binding of the reference peptide (IC₅₀).

Proteasomal Cleavage of the HA-1 Polymorphic Region.

Twenty-nine amino acid long HA-1^{H} and HA-1^{R} peptides were purified to >95% by reverse phase HPLC. 10 μg/ml of the peptides were incubated with 20S proteasomes isolated from EBV-LCLs for 15 min, 30 min, 45 min as described elsewhere (69-71). The proteolysis products were analyzed by tandem mass spectrometry, as described (72).

Dendritic Cell Culture.

Monocyte derived DCs (MoDCs) were generated from healthy individuals by culturing peripheral blood derived CD14⁺ monocytes with 1000 U/ml IL-4 (Genzyme, Cambridge, Mass., USA) and 800 U/ml GMCSF (donated by Dr. S. Osanto, LUMC, Leiden, The Netherlands) for 6 days as described elsewhere (73). On day 6, the DCs were maturated by culturing on irradiated (750 gy) CD40L transfected mouse fibroblasts at a DC to fibroblast ratio of 2:1 or by adding 50% of monocyte conditioned medium (73). Mature DCs were pulsed with HA-1 peptides for 2 hours at 37° C. in Aim-V medium prior to their use as stimulator cells.

In Vitro Induction of HLA-B60/HA-1 Specific T Cell Responses.

Peptide pulsed DCs were cocultured with autologous PBMC at a DC to PBMC ratio of 1:10 in IMDM, 10% human serum supplemented with 1 U/ml IL-2 (Cetus, Emeryville, Calif., USA) and 1 U/ml IL-12 (R & D systems, Minneapolis, Minn., USA). On day 5, 20 U/ml IL-2 was added. On day 7, the T cell lines (TCL) were depleted of CD4⁺ cells using immunomagnetic beads (Dynal AS, Oslo, Norway) and were restimulated with irradiated (150 Gy) peptide pulsed mature DCs (DC:T cell ratio 1:10) or with irradiated (150 Gy) peptide pulsed monocytes (monocyte:T ratio=1:3). Twenty-four hours and 96 hours after restimulation, medium containing 20 U/ml IL-2 was added. TCL were subsequently restimulated every 7 days and were tested for HA-1 specific activity in Interferon-γ (IFN-γ) elispot assays (74) prior to each restimulation.

Results

Effective Binding of Nonameric and Decameric HA-1^{H} and HA-1^{R} Peptides to HLA-B60.

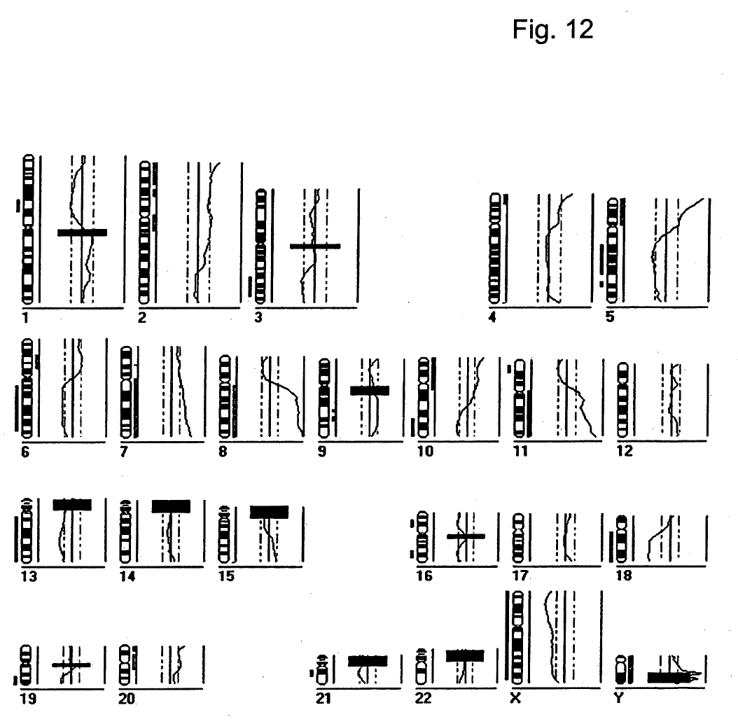

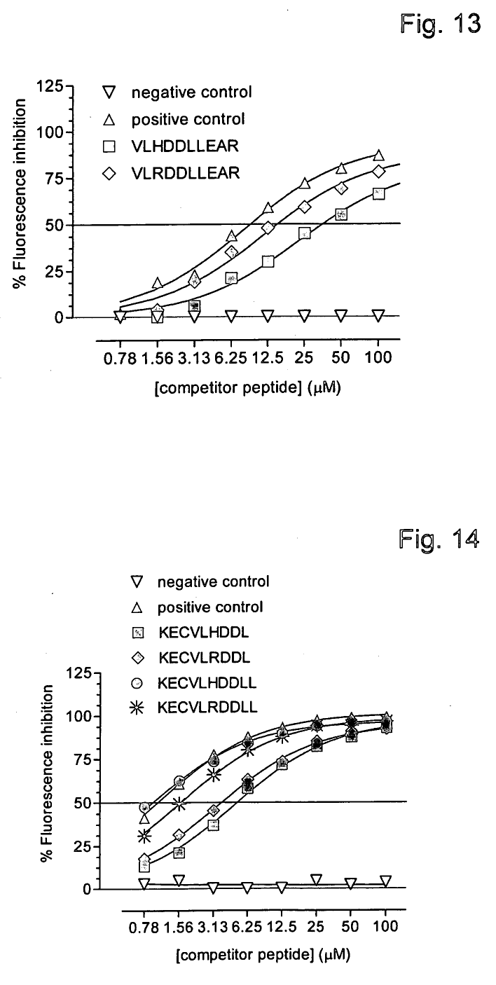

Three categories of HLA molecules were selected for the peptide binding assays: those molecules with a frequency of more than 10% in the Caucasian population, those with binding motifs and those that were predicted to bind nonameric/decameric HA-1^{H/R} peptides. All nonameric HA-1^{H} and HA-1^{R} peptides (n=18) were tested for binding to the so called frequent HLA class I molecules HLA-AL, -A2, -A3, -A11, -A24, -B7, -B8, -B35, -B62. The peptide analysis was extended with two decameric HA-1H/R peptides with a binding motif for HLA-A3 and with five nonameric/decameric peptides predicted to bind either to HLA-B14 or -B60 (table 3). The HLA-A1, -A11, -A24, -B7, -B8, -B114, B35 and -B62 molecules did not bind nonameric HA-1^{H/R} peptides, despite the predictions of BIMAS software for intermediate to strong binding of peptide ECVLRDDLL to HLA-B8 or to -B14 (table 3). The decameric HA-1^{H/R} peptides VL^{H}/_{R}DDLLEAR showed weak to intermediate binding to HLA-A3 molecules with IC₅₀ values of 15.6 μM and 37.5 μM respectively (FIG. 13). In agreement with the prediction of the BIMAS software, the nonameric and decameric HA-1^{H/R} peptides KECVLHDDL, KECVLRDDL, KECVLHDDLL and KECVLRDDLL showed strong binding to HLA-B60 molecules with very low IC₅₀ values of 5.3 μM, 3.9 μM, 1.0 μM and 1.6 μM respectively (FIG. 14). As expected, the original HLA-A2/HA-1^{H} CTL epitope, also predicted by the BIMAS software, displayed binding to HLA-A2 with an IC₅₀ value of 6.4 μM (data not shown).

Stable Binding of Nonameric and Decameric HA-1^{H} and HA-1^{R} Peptides to HLA-B60

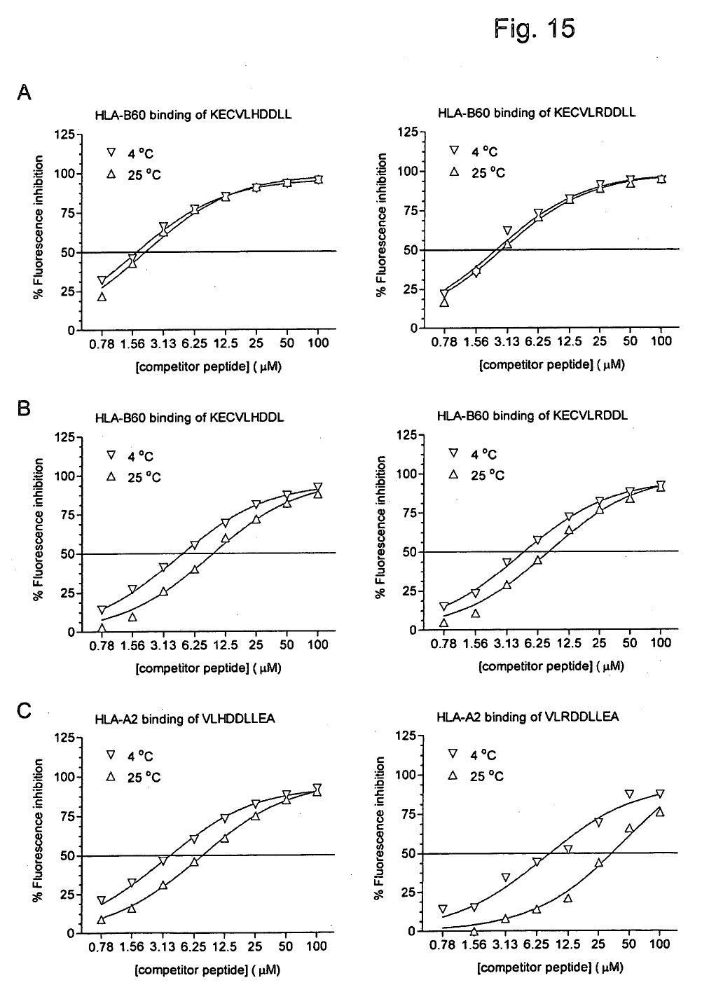

The stability of the HLA-B60/HA-1^{H/R} peptide binding was addressed by testing for the HLA peptide binding capacities at 4° C. and 25° C. HLA-A2/HA-1^{H/R} peptide binding stability was analyzed in parallel as comparison. Increasing the temperature from 4° C. to 25° C. did not affect the strong binding of decameric HA-1^{H/R} peptides to HLA-B60 (FIG. 15a). Less binding was observed with the nonameric HA-1^{H/R} peptides to HLA-B60 (FIG. 15b) which was comparable to the nonameric HA-1^{H} peptide to HLA-A2 (FIG. 15c). Increasing the temperature from 4° C. to 25° C. further decreased the intermediate binding of the nonameric HA-1^{R} peptide to HLA-A2 (FIG. 15c). Thus, the binding of both HA-1^{H} and HA-1^{R} peptides to HLA-B60 were stable and not temperature sensitive.

Proper Proteasomal Cleavage of the HLA-B60 Binding HA-1^{H/R} Peptides

Twenty-nine amino acid long HA-1^{H/R} peptides were subjected to in vitro digestion with EBV-LCL derived 20S immuno-proteasomes. Within a time frame of 15 minutes, major peptide fragments were cleaved at the COOH-termini of both nonameric and decameric HLA-B60 binding HA-1^{H/R} peptides. The latter cleavage products contained the intact HLA-B60 binding sequences with 3-5 additional amino acid residues at the N termini for the HA-1^{H} and HA-1^{R} peptides as demonstrated in Table 4 and Table 5, respectively. Thus, both the HA-1^{H} and the HA-1^{R} products can be effectively cleaved by proteasomes to generate the precursors of the peptides that bind to HLA-B60.

In Vitro Induction of HLA-B60 Restricted T Cells Against the Nonameric HA-1^{H} Peptide.

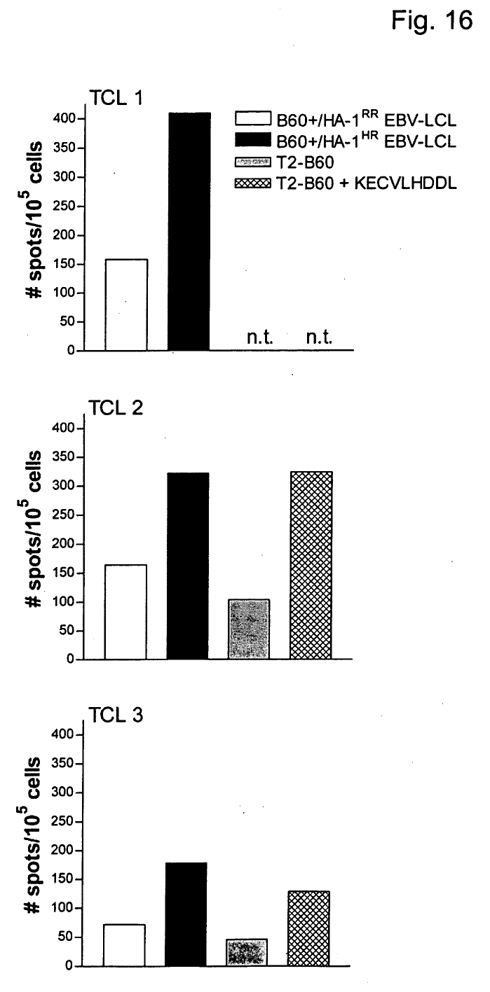

To test the immunogenicity of both the HA-1^{H} and the HA-1^{R} peptides in the context of HLA-B60, PBMCs from three HLA-B60⁺ HA-1^{RR} and from two HLA-B60⁺ HA-1^{HH} healthy individuals were stimulated with autologous DCs pulsed with the nonameric HA-1 ^{H} or HA-1^{R} peptide respectively. After two or three rounds of stimulation, the two T cell lines (TCL) induced with the HA-1^{R} peptide contained significant numbers of IFN-γ producing T cells that recognized HA-1^{R} peptide pulsed HLA-B60 transfected T2 cells. Nevertheless, neither TCL induced with HA-1^{R} peptide produced IFN-γ upon stimulation with EBV-LCLs that express the natural ligand HLA-B60/HA-1^{R} (data not shown). On the contrary, all three TCL induced with the HA-1^{H} peptide contained besides HA-1 nonspecific T cells, significant number of T cells that produced IFN-γ not only upon stimulation with HA-1^{H} peptide pulsed HLA-B60 transfected T2 cells but also upon stimulation with EBV-LCLs that express the natural HLA-B60/HA-1^{H} ligand (FIG. 16).

Discussion

In search for novel T cell epitopes in the HA-1^{H/R} polymorphic region, we studied the binding of polymorphic HA-1 peptides to 11 HLA class I molecules and analyzed the proteasomal cleavage sites in the HA-1^{H/R} polypeptides. These analyses suggested novel interactions of the both alleles of the mHag HA-1 locus with HLA-B60 molecules. Both nonameric and decameric HA-1^{H/R} peptides effectively bind to HLA-B60. In vitro proteasomal analysis showed cleavage at the COOH termini of HLA-B60 binding peptides, indicating proper intracellular processing.

Both nonameric and decameric HA-1^{H/R} peptides show strong binding to HLA-B60, with IC₅₀ values between 1.6-5.3 μM. These HLA binding levels are similar to or higher than the HLA binding of the immunogenic HLA-A2/HA-1^{H} CTL epitope and of other reported T cell epitopes measured in similar assays (67, 75). Furthermore, we compared the stability of the HLA-B60/HA-1^{H/R} with HLA-A2/HA-1^{H/R} peptide interactions by increasing the temperature of the binding assays. These assays reveal that unlike the HLA-A2/HA-1^{R} peptide interaction, the HLA-B60/HA1^{H/R} and HLA-A2/HA-1^{H} interactions are stable. The stability of HLA-B60/HA-1^{H/R} interactions were confirmed in separate experiments using fluorescent HA-1^{H/R} peptides (data not shown). Thus, both HA-1^{H} and HA-1^{R} peptides can efficiently interact with HLA-B60, which is an important biochemical feature of strongly immunogenic T cell epitopes (75). This actually predicts immunogenicity of both HA-1^{H} and HA-1^{R} locus products in association with HLA-B60. The HLA peptide binding is preceded by intracellular processing of cellular proteins. In the endoplasmic reticulum (ER), proteasomally cleaved peptides can undergo NH2-terminal trimming by aminopeptidases (76). COOH-terminal trimming in de ER have not been demonstrated. The proper generation of the correct COOH-terminus by an early major cleavage site by proteasomes is thus a key event for efficient epitope generation as demonstrated by recent studies (77-80). In our in vitro cleavage studies, the correct COOH termini of HLA-B60 binding sequences of both the HA- and the HA-1^{R} allele were generated within 15 minutes. These peptide fragments contained the intact HLA-B60 binding sequences. The exact sequences of the HLA-B60 binding peptides were not present as proteasomal degradation products. Also some additional cleavage sites within the putative T cell epitopes were observed. Nonetheless, the successful generation of HLA-B60/HA-1^{H} specific T cells demonstrates the proper cleavage of the HLA-B60 binding HA-1^{H} peptides by cellular antigen processing machinery.

### Example 4

Total RNA was prepared from subconfluent layers of the adherent cell cultures using the RNAzol method (Cinaa/Biotecx Laboratories, Houston, Tex.) according to the manufacturer's description. cDNA was synthesized using 2 mg RNA and random hexameric primers. PCR amplification and quantification were performed using the Taqman PCR assay (PE Applied Biosystems 7700 Sequence Detector, Foster City, Calif.). We used comparative quantification normalizing the HA-1 and CD45 gene to an internal standard gene, the ubiquitously expressed housekeeping gene porphobilinogen deaminase (PBGD). To allow calculation of relative levels of expression, we used the KG-1 cell line, which expresses both genes, as a standard. The HA-1 and CD45 expression levels of the test samples were calculated as percentages of HA-1 and CD45 expression levels in the reference cell line KG-1. All samples tested which showed expression levels below 10% in the real time quantitative PCR did not produce detectable PCR fragments in a standard PCR. Therefore, expression levels <10% are considered as not significant. The relative quantification was calculated by the linear calibration function between the threshold cycle (Ct) value and the logarithm of the initial starting quantity (N) were Ct=−3.31 log (N)+26.1, Ct=−3.5 log(N)+21.6 and Ct=−3.41 log(N)+25.6 for HA-1, CD45 and PBGD, respectively. The HA-1, CD45 and PBGD expression were quantified in all test samples by using these calibration functions.

### Example 5

Tumor cell lines were used as target cells in a 4 hr 51Cr release assay. The tumor cells from subconfluent cultures were harvested and dispensed at 2500 cells/well in 96 wells flat bottomed microtiter plates and allowed to attach either in the presence or the absence of rINFg (250 U/ml, Gentech, San Francisco, Calif.) and TNFa (250 U/ml, San Francisco, Calif.) for 48 hrs. The tumor cells were labeled with 51Cr for 1 hr and the experiments were performed in sixplicates. The percentage specific lysis was calculated as follows: % specific lysis=(experimental release−spontaneous release)/(maximal release−spontaneous release)×100.

### Example 6

Preparation of cryosections. Sections (5 μm) from freshly shock frozen primary tumors were placed on a polyethylene membrane on a glass slide, stained with Meyer's hematoxylin and dehydrated in 70%, 90% and 100% ethanol. The PALM Microbeam system (Bernried, Germany) was used for microdissection and catapulting.

### Example 7

Detection of disseminated cells, global amplification of micro-dissected areas and of single cells from bone marrow and lymph nodes was performed as described in detail (Klein et al., submitted). Briefly, the viable bone marrow or lymph node samples were stained for 10 min. with 10 μg/ml monoclonal antibody 3B10-C9 in the presence of 5% AB-serum. 3B10-C9-positive cells were detected with B-phycoerythrin-conjugated goat antibody to mouse IgG (The Jackson Laboratory) and transferred to PCR-tubes on ice.

Oligo-dT beads in 10 μl lysis buffer (Dynal) were added, the cells lysed, tubes rotated for 30 min. to capture mRNA. 10 μl cDNA wash buffer-1 (50 mM Tris-HCl, pH 8.3, 75 mM KCl, 3 mM MgCl2, 10 mM DTT, supplemented with 0.5% Igepal (Sigma)) was added and mRNA bound to the beads washed in 20 μl cDNA wash buffer-2 (50 mM Tris-HCl, pH 8.3, 75 mM KCl, 3 mM MgCl2, 10 mM DTT, supplemented with 0.5% Tween-20 (Sigma)), transferred to a fresh tube and washed again in cDNA wash buffer-1. mRNA was reverse transcribed with Superscript II Reverse Transcriptase (Gibco BRL) using the buffers supplied by the manufacturer supplemented with 500 μM dNTP, 0.25% Igepal, 30 μM CFL5c8 primer (5′-(CCC)5 GTC TAG ANN(N)8-3′ (SEQ ID NO:______) and 15 μM CFL5cT (5′-(CCC)5 GTC TAG ATT (TTT)4 TVN (SEQ ID NO:______), at 44° C. for 45 min. Samples were rotated during the reaction to avoid sedimentation of the beads. cDNA remained linked to the paramagnetic beads via the mRNA and was washed once in the tailing wash buffer (50 mM KH2PO4, pH 7.0, 1 mM DTT, 0.25% Igepal). Beads were resuspended in tailing buffer (10 mM KH2PO4, pH 7.0, 4 mM MgCl2, 0.1 mM DTT, 200 μM GTP) and cDNA-mRNA hybrids were denatured at 94° C. for 4 min, chilled on ice, 10 U TdT (MBI-Fermentas) added and incubated at 37° C. for 30-60 min. After inactivation of the tailing enzyme (70° C., 5 min), PCR-Mix I was added consisting of 4 μl of buffer 1 (Roche, Taq long template), 3% deionized formamide (Sigma) in a volume of 35 μl. The probes were heated at 78° C. in the PCR cycler (Perkin Elmer 2400), PCR Mix II, containing dNTPs at a final concentration of 350 μM, CP2 primer (5′-TCA-GAA-TTC-ATG-CCC-CCC-CCC-CCC-CCC-3′ (SEQ ID NO:______), final concentration 1.2 μM) and 5 Units of the DNA Poly-Mix was added, (Roche, Taq Long Template) in a volume of 5 μl for a hot start procedure. Forty cycles were run at 94° C. for 15 sec, at 65° C., 30° C., 68° C. for 2 min. for the first 20 cycles and a 10 sec-elongation of the extension time each cycle for the remaining 20 cycles, and a final extension step at 68° C., 7 min

For expression profiling digoxigenin-UTP was incorporated by PCR using 0.1-1 μl of the original PCR amplified cDNA fragments reamplification in the presence of 50 μM dig-dUTP (Roche), 300 μM dTTP, and other dNTPs at a final concentration of 350 μM. Reamplification conditions were essentially as described above, modifications were the use of 2.5 Units of the DNA Poly Mix. Initial denaturation at 94° C. for 2 min. followed by 12 cycles at 94° C., 15 sec, 68° C., 3 min. and a final extension time of 7 min. Filters were pre-hybridized overnight in the presence of 50 mg/ml E. coli and 50 mg/ml pBS DNA in 6 ml Dig-easy Hyb buffer (Roche). Labeled PCR products from single cells were added in a concentration of 1.5 μg/ml mixed with 100 ptg herring sperm to prehybridization buffer, and hybridized for 36-48 hours. Stringency washes were performed according to the Roche™ digoxigenin hybridization protocol adding two final stringency washes in 0.1×SSC+0.1% SDS for 15 min at 68° C. Detection of filter bound probes was performed according to the digoxigenin detection system protocol supplied with the kit (Roche).

### Example 8

Amplification of HA-1 and CD45. All samples were analyzed by two primer pairs for HA-1: HA-1 (I) (forward: 5′-GAC GTC GTC GAG GAC ATC TCC CAT-3′; reverse: 5′-GAA GGC CAC AGC AAT CGT CTC CAG-3′ (SEQ ID NO:______) and HA-1 (II) (forward: 5′-ACA CTG CTG TCG TGT GAA GTC-3′ (SEQ ID NO:______)); reverse: 5′-TCA GGC CCT GCT GTA CTG CA-3′ (SEQ ID NO:______)). CD45 forward: 5′-CTG AAG GAG ACC ATT GGT GA (SEQ ID NO:______)) and reverse 5′-GGT ACT GGT ACA CAG TTC GA-3′ (SEQ ID NO:______) primer. Amplification products of the HA-1 (I) primers were digested with the restriction enzyme BstU I and amplification products of the HA-1 (II) primers with Hinf I. Southern blot was performed according to standard protocols.

TABLE 1
HA-1 and CD45 gene expression in tumor cell lines. Percentages
represent HA-1 and CD45 gene expression relative to the standard cell
line KG-1 as analyzed by quantitative real time PCR.

| Tumor type | Cell line | % CD45 | % HA-1 |
|---|---|---|---|
| Breast cancer | ZR75- 1 | ≦10 | 54 |
| | BT-20 | ≦10 | 40 |
| | 734B | ≦10 | 27 |
| | T47 D | ≦10 | 17 |
| | MDA-MB231 | ≦10 | 15 |
| | MCF-7 | ≦10 | ≦10 |
| | BT474 | ≦10 | ≦10 |
| Melanoma | Mel 93.04 | ≦10 | 68 |
| | KUL 68/3636 | ≦10 | 67 |
| | BB 74/2940 | ≦10 | 57 |
| | MNT | ≦10 | 27 |
| | LB33 | ≦10 | 24 |
| | BT | ≦10 | 15 |
| | 453 Ao | ≦10 | 12 |
| | 518A | ≦10 | ≦10 |
| | E9 | ≦10 | ≦10 |
| | MEWO | ≦10 | ≦10 |
| Lung carcinoma | GLC 36 | ≦10 | 22 |
| | GLC 8 | ≦10 | ≦10 |
| | GLC 2 | ≦10 | ≦10 |
| Renal Cell Carcinoma | MZ 1851 | ≦10 | 29 |
| | MZ 1752 | ≦10 | 13 |
| | MZ 1774 | ≦10 | ≦10 |
| | BA | ≦10 | ≦10 |
| Hepatoma | HuH7 | ≦10 | 37 |
| | HepG2 | ≦10 | 35 |
| Colon carcinoma | SW 707 | ≦10 | 147 |
| | CaCo-2 | ≦10 | 81 |
| | SW 480 | ≦10 | 70 |
| | SW 2219 | ≦10 | 48 |
| | SW 620 | ≦10 | 28 |
| | Col 205 | ≦10 | 21 |
| | SW 948 | ≦10 | 12 |
| | HT29 | ≦10 | 11 |
| Head and Neck cancer | BB 49/ 1413 | ≦10 | 54 |

TABLE 2
CTL recognition of tumor cell lines. The results are given as percentage specific
lysis (Example 5) by one allo HLA-A2 and by two HA-1 specific CTL clones at
different effector (E) to target (T) ratio's.

| | | | % specific lysis by | | | | | |
|---|---|---|---|---|---|---|---|---|
| | | | | | HA-1 CTLs | | | |
| | | | HLA-A2 CTLs | | 5W38 | | 3HA15 | |
| Tumor cell lines | | | IFN_{γ}/TNF_{α} | | IFN_{γ}/TNF_{α} | | IFN_{γ}/TNF_{α} | |
| Tumor type | Designation | E:T | no | yes | no | yes | no | yes |
| Breast cancer | MDA-MB 231 | 2:1 | 10 | 13 | 8 | 15 | 8 | 13 |
| | | 10:1 | 50 | 64 | 31 | 47 | 25 | 39 |
| Melanoma | MEL 93.04 | 2:1 | 10 | 14 | 1 | 13 | −2 | 10 |
| | | 10:1 | 54 | 64 | 12 | 37 | 17 | 40 |
| Melanoma | 453 A0 | 2:1 | 7 | 24 | 1 | 10 | 1 | 18 |
| | | 10:1 | 25 | 43 | 5 | 21 | 2 | 22 |
| | | 20:1 | 35 | 45 | 7 | 24 | 7 | 21 |
| Lung carcinoma | GLC 36 | 1:1 | 33 | 35 | 6 | 12 | 0 | 8 |
| | | 10:1 | 59 | 80 | 8 | 25 | 17 | 25 |
| Colon carcinoma | CaCo-2 | 1.6:1 | 20 | 22 | 1 | 2 | 6 | 7 |
| | | 16:1 | 29 | 49 | 4 | 4 | 11 | 17 |

TABLE 3
Peptides of the HA-1 polymorphic region tested for
binding to different HLA class I molecules.

| | HA-1^{S/R} polymorphic region sequence | | | | | | | | | | | | | | | | | Binding predicted to | |
|---|---|---|---|---|---|---|---|---|---|---|---|---|---|---|---|---|---|---|---|
| Peptide# | E | K | L | K | E | C | V | L | H/R | D | D | L | L | E | A | R | R | (BIMAS store) ^{a} | |
| 1 | E | K | L | K | E | C | V | L | H | | | | | | | | | | |
| 2 | E | K | L | K | E | C | V | L | R | | | | | | | | | | |
| 3 | | K | L | K | E | C | V | L | H | D | | | | | | | | | |
| 4 | | K | L | K | E | C | V | L | R | D | | | | | | | | | |
| 5 | | | L | K | E | C | V | L | H | D | D | | | | | | | | |
| 6 | | | L | K | E | C | V | L | R | D | D | | | | | | | | |
| 7 | | | | K | E | C | V | L | H | D | D | L | | | | | | HLA-B60 | (176) |
| 8 | | | | K | E | C | V | L | R | D | D | L | | | | | | HLA-B60 | (176) |
| 9 | | | | K | E | C | V | L | H | D | D | L | L | | | | | HLA-B60 | (160) |
| 10 | | | | K | E | C | V | L | R | D | D | L | L | | | | | HLA-B60 | (160) |
| 11 | | | | | E | C | V | L | H | D | D | L | L | | | | | | |
| 12 | | | | | E | C | V | L | R | D | D | L | L | | | | | HLA-B8 | (32), |
| | | | | | | | | | | | | | | | | | | -B14 | (90) |
| 13 | | | | | | C | V | L | H | D | D | L | L | E | | | | | |
| 14 | | | | | | C | V | L | R | D | D | L | L | E | | | | | |
| 15 | | | | | | | V | L | H | D | D | L | L | E | A | | | HLA-A2 | (79.6) |
| 16 | | | | | | | V | L | R | D | D | L | L | E | A | | | | |
| 17 | | | | | | | V | L | H | D | D | L | L | E | A | R | | HLA-A3 | |
| 18 | | | | | | | V | L | R | D | D | L | L | E | A | R | | HLA-A3 | |
| 19 | | | | | | | | L | H | D | D | L | L | E | A | R | | | |
| 20 | | | | | | | | L | R | D | D | L | L | E | A | R | | | |
| 21 | | | | | | | | | H | D | D | L | L | E | A | R | R | | |
| 22 | | | | | | | | | R | D | D | L | L | E | A | R | R | | |

Peptides# 1-8, 11-16 and 19-22 were assayed for binding to HLA-A1, -A2, -A3, -A11, -A24, -B7, -B8, -B35, -B62 regardless of prediction.
^{a} Prediction of HLA/peptide associations was executed using BIMAS software except for peptides 17 and 18, which were not predicted by BIMAS but contain HLA-A3 anchor amino acids at position 2 and 10.

TABLE 4
In vitro proteasomal cleavage of a 29 amino acid long HA-1^{A} peptide

| | | | G L E K L K E C V L H D D L L E A R R P R A H E C L G E A |
|---|---|---|---|
| % fragment digested | | | |
| in | | | |
| 15 min | 30 min | 45 min | |
| 17.2 | 22.4 | 0 | G L E K L K E C V L K D D L |
| 14.7 | 11.8 | 14.7 | G L E K L K E C V L H D D L L E A R R P R A H E C L G |
| 13.9 | 16.4 | 21.7 | H D D L L E A R R P R A H E C L G E A |
| 13.0 | 10.3 | 13.9 | G L E K L K E C V L H D D L L E A R R P R A |
| 10.5 | 8.3 | 12.1 | E K L K E C V L H D D L L |
| 9.6 | 8.8 | 12.3 | G L E K L K E C V L H D D L L E A R R P R A H E C |
| 8.5 | 9.2 | 13.8 | G L E K L K E C V L H D |
| 7.8 | 7.4 | 11.5 | G L E K L K E C V L H D D L L E A |
| 4.8 | 5.5 | 0 | G L E K L K E C V L |

The peptide sequences that bind to HLA-B60 are underlined. The proteolytic fragments cleaved at the COOH termini of the ULA-B60 binding peptides are indicated in hold. The amounts of the generated fragments after cleavage with 20s imniuno proteasomes for 15, 30 and 45 min are expressed an the percentage of all fragments found in the digested substrate.

TABLE 5
In vitro proteasomal cleavage of a 29 amino acidlong HA-1^{R} peptide

| | | | G L E K L K E C V L R D D L L E A R R P R A H E C L G E A |
|---|---|---|---|
| % fragment digested | | | |
| in | | | |
| 15 min | 30 min | 45 min | |
| 26.2 | 28.0 | 23.8 | G L E K L K E C V L R D D L L E A R R P R A H E C L G |
| 14.0 | l6.0 | 13.5 | G L E K L K E C V L R D D L L E A R R P R A H E C L G E |
| 11.1 | 14.3 | 12.7 | G L E K L K E C V L R D D L |
| 7.9 | 9.6 | 7.9 | G L E K L K E C V L R D D L L E A R R P R A |
| 6.6 | 8.6 | 8.3 | E K L K E C V L R D D L L |
| 6.2 | 7.5 | 7.3 | C V L R D D L L E A R R |
| 5.3 | 7.0 | 5.7 | G L E K L K E C V L R D D L L E A R R P R A H E C L |
| 4.9 | 7.1 | 6.4 | G L E K L K E C V L R D D L L E A R R P R A H E C |
| 4.2 | 6.1 | 5.6 | G L E K L K E C V L R D |
| 3.9 | 4.2 | 3.9 | G L E K L K E C V L R D D L L E A |
| 3.6 | 4.4 | 4.4 | G L E K L K E C V L R D D L L E A R R |
| 3.4 | 4.2 | 3.6 | G L E K L K E C V L R D D L L E A R R P R |
| 2.6 | 4.2 | 4.0 | G L E K L K E C V L R D D L L E A R R P R A H |

The peptade sequences that bind to HLA-B60 are underlined. The proteolytic fragments cleaved at the COOH termini of the HLA-B60 binding peptides are indicated in bold. The amounts of the generated fragments after cleavage with 20s immuno proteasomes for 15, 30 and 45 min are expressed as the percentage of all fragments found in the digested substrate.

TABLE 6

| | CTL analysis | | Nr. of clones | DNA analysis |
|---|---|---|---|---|
| Cell | HA-1 phenotype | KIAA0223 sequence | sequenced | HA-1 phenotype |
| DH | HA-1−/− | GAGTGTGTGTTGCGTGACGACCTCCTTGAGGCCCGCCG | (6/6 clones) | HA-1^{R}/HA-1^{R} |
| | | E C V L R D D L L E A R R | | |
| vR | HA-1+/+ | GAGTGTGTGCTGCATGACGACCTCCTTGAGGCCCGCCG | (6/6 clones) | HA-1^{H}/HA-1^{H} |
| | | E C V L H D D L L E A R R | | |
| KG-1 | HA-1+ | GAGTGTGTGTTGCGTGACGACCTCCTTGAGGCCCGCCG | (1/8 clones) | HA-1^{R}/HA-1^{H} |
| | | E C V L R D D L L E A R R | | |
| | | GAGTGTGTGCTGCATGACGACCTCCTTGAGGCCCGCCG | (7/8 clones) | |
| | | E C V L H D D L L E A R R | | |

### REFERENCES

1. Beatty, P. G. et al. Marrow transplantation from HLA-matched unrelated donors for treatment of hematologic malignancies. Transplantation 51, 443-447 (1997).

2. Marks, D. I. et al. Allogeneic bone marrow transplantation for chronic myeloid leukemia using sibling and volunteer unrelated donors. A comparison of complications in the first 2 years. Ann. Intern. Med. 119, 207-214 (1993).

3. Goulmy, E. et al. Mismatches of minor histocompatibility antigens between HLA-identical donors and recipients and the development of graft-versus-host disease after bone marrow transplantation. N. Engl. J. Med. 334, 281-285 (1996).

4. den Haan, J. M. et al. Identification of a graft versus host disease-associated human minor histocompatibility antigen. Science 268, 1476-1480 (1995).

5. Wang, W. et al. Human H-Y: a male-specific histocompatibility antigen derived from the SMCY protein [see comments]. Science 269, 1588-1590 (1995).

6. Meadows, L. et al. The HLA-A*0201-restricted H-Y antigen contains a posttranslationally modified cysteine that significantly affects T cell recognition. Immunity. 6, 273-281 (1997).

7. van Els, C. A. et al. Immunogenetics of human minor histocompatibility antigens: their polymorphism and immunodominance. Immunogenetics 35, 161-165 (1992).

8. de Bueger, M., Bakker, A., van Rood, J. J., Van der Woude, F. & Goulmy, E. Tissue distribution of human minor histocompatibility antigens. Ubiquitous versus restricted tissue distribution indicates heterogeneity among human cytotoxic T lymphocyte-defined non-MHC antigens. J. Immunol. 149, 1788-1794 (1992).

9. Van Lochem, E., van der Keur, M., Mommaas, A. M., de Gast, G. C. & Goulmy, E. Functional expression of minor histocompatibility antigens on human peripheral blood dendritic cells and epidermal Langerhans cells. Transpl. Immunol. 4, 151-157 (1996).

10. van der Harst, D. et al. Recognition of minor histocompatibility antigens on lymphocytic and myeloid leukemic cells by cytotoxic T-cell clones. Blood 83, 1060-1066 (1994).

11. Schreuder, G. M. et al. A genetic analysis of human minor histocompatibility antigens demonstrates Mendelian segregation independent of HLA. Immunogenetics 38, 98-105 (1993).

12. Goulmy, E., Pool, J. & van den Elsen, P. J. Interindividual conservation of T-cell receptor beta chain variable regions by minor histocompatibility antigen-specific HLA-A*0201-restricted cytotoxic T-cell clones. Blood 85, 2478-2481 (1995).

13. Ruppert, J., Sidney, J., Celis, E., Kubo, R. T., Grey, H. M. & Sette, A. Prominent role of secondary anchor residues in peptide binding to HLA-A2.1 molecules. Cell 74, 929-937 (1993).

14. Chen, Y. et al. Naturally processed peptides longer than nine amino acid residues bind to the class I MHC molecule HLA-A2.1 with high affinity and in different conformations. J. Immunol. 152, 2874-2881 (1994).

15. Loveland, B., Wang, C. R., Yonekawa, H., Hermel, E. & Lindahl, K. F. Maternally transmitted histocompatibility antigen of mice: a hydrophobic peptide of a mitochondrially encoded protein. Cell 60, 971-980 (1990).

16. Loveland, B. E., Fischer Lindahl, K. The definition and expression of minor histocompatibility antigens. McCluskey J, editors. Antigen processing and recognition. London: CRC Press, 9, 173-92 (1991)

17. Lindahl, K. F. Minor histocompatibility antigens. Trends. Genet. 7, 219-224 (1991).

18. Perreault, C., Jutras, J., Roy, D. C., Filep, J. G. & Brochu, S. Identification of an immunodominant mouse minor histocompatibility antigen (MiHA). T cell response to a single dominant MiHA causes graft-versus-host disease. J. Clin. Invest. 98, 622-628 (1996).

19. Morse, M. C. et al. The COI mitochondrial gene encodes a minor histocompatibility antigen presented by H2-M3. J. Immunol. 156, 3301-3307 (1996).

20. Scott, D. M. et al. Identification of a mouse male-specific transplantation antigen, H-Y. Nature 376, 695-698 (1995).

21. Greenfield, A. et al. An H-YDb epitope is encoded by a novel mouse Y chromosome gene. Nat. Genet. 14, 474-478 (1996).

22. Martin, P. J. Increased disparity for minor histocompatibility antigens as a potential cause of increased GVHD risk in marrow transplantation from unrelated donors compared with related donors. Bone Marrow Transplant. 8, 217-223 (1991).

23. Goulmy, E., Gratama, J. W., Blokland, E., Zwaan, F. E. & van Rood, J. J. A minor transplantation antigen detected by MHC-restricted cytotoxic T lymphocytes during graft-versus-host disease. Nature 302, 159-161 (1983).

24. de Bueger, M. et al. Isolation of an HLA-A2.1 extracted human minor histocompatibility peptide. Eur. J. Immunol. 23, 614-618 (1993).

25. Hunt, D. F. et al. Characterization of peptides bound to the class I MHC molecule HLA-A2.1 by mass spectrometry. Science 255, 1261-1263 (1992).

26. Ottenhoff, T. H. M., Geluk, A., Toebes, M., Benckhuijsen, W. E., van Meijgaarden, K. E. & Drijfhout, J. W. A sensitive fluorometric assay for quantitatively measuring specific peptide binding to HLA class I and class II molecules. J. Immunol. Methods 200, 89-97 (1997).

27. Tan, T. L. R., Geluk, A., Toebes, M., Ottenhoff, T. H. M. & Drijfhout, J. W. A novel, highly efficient peptide-HLA class I binding assay using unfolded heavy chain molecules; identification of HIV-1 derived peptides that bind to HLA-A*0201 and HLA-A*0301. Submitted (1997).

28. Traversari, C. et al. Transfection and expression of a gene coding for a human melanoma antigen recognized by autologous cytolytic T lymphocytes. Immunogenetics 35, 145-152 (1992).

29. O Reilly, R. J. Allogeneic bone marrow transplantation: Current status and future directions. Blood 62: 941-964 (1983)

30. Horowitz, M. M. et. al. Graft-versus-leukemia reactions after bone marrow transplantation. Blood 75: 555-562.(1990)

31. Ringden, O. et. al. Allogeneic bone marrow transplantation for leukemia: factors of importance for long-term survival and relapse. Bone Marrow Transplant 3: 281-290.(1988)

32. Kolb, H. J. and Holler, E. Adoptive immunotherapy with donor lymphocyte transfusions. Curr Opin Oncol 9: 139-145.(1997)

33. Kolb, H. J. et. al. Graft-versus-leukemia effect of donor lymphocyte transfusions in marrow grafted patients. European Group for Blood and Marrow Transplantation Working Party Chronic Leukemia. Blood 86: 2041-2050.(1995)

34. Gratwohl, A. et. al. Acute graft-versus-host disease: grade and outcome in patients with chronic myelogenous leukemia. Working Party Chronic Leukemia of the European Group for Blood and Marrow Transplantation. Blood 86: 813-818.(1995)

35. Goulmy, E. Human minor histocompatibility antigens: new concepts for marrow transplantation and adoptive immunotherapy. Immunol Rev 157: 125-140.(1997)

36. Niederwieser, D. et. al. Correlation of minor histocompatibility antigen-specific cytotoxic T lymphocytes with graft-versus-host disease status and analyses of tissue distribution of their target antigens. Blood 81: 2200-2208.(1993)

37. Faber, L. M. et. al. Recognition of clonogenic leukemic cells, remission bone marrow and HLA-identical donor bone marrow by CD8+ or CD4+ minor histocompatibility antigen-specific cytotoxic T lymphocytes. J Clin Invest 96: 877-883.(1995)

38. Falkenburg, J. H. F. et. al. Growth inhibition of clonogenic leukemic precursor cells by minor histocompatibility antigen-specific cytotoxic T lymphocytes. J Exp Med 174: 27-33.(1991)

39. den Haan, J. M. et. al. The minor histocompatibility antigen HA-1: a diallelic gene with a single amino acid polymorphism. Science 279: 1054-1057.(1998)

40. Banchereau, J. and Steinman, R. M. Dendritic cells and the control of immunity. Nature 392: 245-252.(1998)

41. Bonini, C. et. al. HSV-TK gene transfer into donor lymphocytes for control of allogeneic graft-versus-leukemia [see comments]. Science 276: 1719-1724.(1997)

42. Heslop, H. E. et. al. Long-term restoration of immunity against Epstein-Barr virus infection by adoptive transfer of gene-modified virus-specific T lymphocytes. Nature Medicine 2: 551-555.(1996)

43. van Lochem, E. G., Schreuder, G. M., Tilanus, M. G., de Gast, G. C., and Goulmy, E. Dendritic cells induce HLA-DP-specific T-cell proliferation between MLR-negative siblings. Immunogenetics 41: 134-138.(1995)

44. A. Butturini and R. P. Gale, Bone Marrow Transplant. 3, 185 (1988).

45. R. Childs et al., N. Engl.J.Med. 343, 750 (2000).

46. B. Eibl et al., Blood 88, 1501 (1996).

47. R. Ben Yosef, R. Or, A. Nagler, S. Slavin, Lancet 348, 1242 (1996).

48. A. N. Houghton, M. L. Meyers, P. B. Chapman, Surg.Clin.North Am. 76, 1343 (1996).

49. J. O. Bay et al., Bone Marrow Transplant 25, 681 (2000).

50. E. Goulmy, Curr.Opin.Immunol. 8, 75 (1996).

51. D. van der Harst et al., Blood 83, 1060 (1994).

52. J. M. M. den Haan et al., Science 279, 1054 (1998).

53. C. A. Klein et al., Proc.Natl.Acad.Sci.U.S.A 96, 4494 (1999).

54. H. J. Kolb et al., Blood 76, 2462(1990).

55. S. Slavin et al., Blood 91, 756 (1998).

56. T. Mutis et al., Blood 93, 2336 (1999).

57. N. T. Ueno et al., J.Clin.Oncol. 16, 986 (1998).

58. S. Braun et al., N.Engl.J.Med. 342, 525 (2000).

59. The following cell lines were kindly provided by: MDA-MB 231, 734 B, MCF-7, ZR75-1 by Dr. B. Eibl (Dept. of Clinical Immunobiology, University Hospital of Internal Medicine, Innsbruck, Austria); HBL-100 cell line, Mel 93.04C and LB 33 by Dr. S. Osanto (Dept. of Oncology, Leiden University Medical Center, Leiden, The Netherlands); BT-20, BT, MEWO, E9, BT, MNT and BA by Prof. G. C. de Gast (UMC, Utrecht, The Netherlands); GLC2, GLC 8, and GLC 36 by Prof. L. de Leij (Dept. of Clinical Immunology, AZG, Groningen, The Netherlands); BB74/2940, KUL 68/3636 and BB 49/1413 by Dr. F. Brasseur, (Ludwig Institute for Cancer Research, Brussels, Belgium); HuH7 and HepG2 by Dr. B. J. Scholte (Erasmus University Rotterdam, The Netherlands); HT-29 (ATCC: HTB-38) and Caco-2 (ATCC: HTB-37) are ATCC cell lines.

60. Goulmy, E. 1996. Human minor histocompatibility antigens. Curr.Opin.Immunol. 8:75.

61. den Haan, J. M., L. M. Meadows, W. Wang, J. Pool, E. Blokland, T. L. Bishop, C. Reinhardus, J. Shabanowitz, R. Offringa, D. F. Hunt, V. H. Engelhard, and E. Goulmy. 1998. The minor histocompatibility antigen HA-1: a diallelic gene with a single amino acid polymorphism. Science 279:1054.

62. Goulmy, E., J. W. Gratama, E. Blokland, F. E. Zwaan, and J. J. Van Rood. 1983. A minor transplantation antigen detected by MHC-restricted cytotoxic T lymphocytes during graft-versus-host disease. Nature 302:159.

63. Mutis, T., R. Verdijk, E. Schrama, B. Esendam, A. Brand, and E. Goulmy. 1999. Feasibility of inmmunotherapy of relapsed leukemia with ex vivo-generated cytotoxic T lymphocytes specific for hematopoietic system-restricted minor histocompatibility antigens. Blood 93:2336.

64. Goulmy, E. 1997. Human minor histocompatibility antigens: new concepts for marrow transplantation and adoptive immunotherapy. Immunol.Rev. 157:125.

65. Mendoza, L. M., P. Paz, A. Zuberi, G. Christianson, D. Roopenian, and N. Shastri. 1997. Minors held by majors: the H13 minor histocompatibility locus defined as a peptide/MHC class I complex. Immunity. 7:461.

66. Ostrov, D. A., M. M. Roden, W. Shi, E. Palmieri, G. J. Christianson, L. Mendoza, G. Villaflor, D. Tilley, N. Shastri, H. Grey, S. C. Almo, D. Roopenian, and S. G. Nathenson. 2002. How H13 histocompatibility peptides differing by a single methyl group and lacking conventional MHC binding anchor motifs determine self-nonself discrimination. J.Immunol. 168:283.

67. van der Burg, S. H., E. Ras, J. W. Drijfhout, W. E. Benckhuijsen, A. J. Bremers, C. J. Melief, and W. M. Kast. 1995. An HLA class I peptide-binding assay based on competition for binding to class I molecules on intact human B cells. Identification of conserved HIV-1 polymerase peptides binding to HLA-A*0301. Hum.Immunol. 44:189.

68. Rammensee H. G., J. Bachmann, and S. Stevanovic. 1997. MHC ligands and peptide motifs. Springer-Verlag NJ, Landes Bioscience Austin, Tex.

69. Groettrup, M., T. Ruppert, L. Kuehn, M. Seeger, S. Standera, U. Koszinowski, and P. M. Kloetzel. 1995. The interferon-gamma-inducible 11 S regulator (PA28) and the LMP2/LMP7 subunits govern the peptide production by the 20 S proteasome in vitro. J. Biol. Chem. 270:23808.

70. Frisan, T., V. Levitsky, A. Polack, and M. G. Masucci. 1998. Phenotype-dependent differences in proteasome subunit composition and cleavage specificity in B cell lines. J.Immunol. 160:3281.

71. Eggers, M., B. Boes-Fabian, T. Ruppert, P. M. Kloetzel, and U. H. Koszinowski. 1995. The cleavage preference of the proteasome governs the yield of antigenic peptides. J.Exp.Med. 182:1865.

72. Kessler, J. H., N. J. Beekman, S. A. Bres-Vloemans, P. Verdijk, P. A. van Veelen, A. M. Kloosterman-Joosten, D. C. Vissers, G. J. ten Bosch, M. G. Kester, A. Sijts, J. W. Drijfhout, F. Ossendorp, R. Offringa, and C. J. Melief. 2001. Efficient Identification of Novel HLAA*0201-presented Cytotoxic T Lymphocyte Epitopes in the Widely Expressed Tumor Antigen PRAME by Proteasome-mediated Digestion Analysis. J.Exp.Med. 193:73.

73. Verdijk, R. M., T. Mutis, B. Esendam, J. Kamp, C. J. Melief, A. Brand, and E. Goulmy. 1999. Polyriboinosinic polyribocytidylic acid (poly(I:C)) induces stable maturation of functionally active human dendritic cells. J.Immunol. 163:57.

74. Schmittel, A., U. Keilholz, and C. Scheibenbogen. 1997. Evaluation of the interferon-gamma ELISPOT-assay for quantification of peptide specific T lymphocytes from peripheral blood. J.Immunol.Methods 210:167.

75. van der Burg, S. H., M. J. Visseren, R. M. Brandt, W. M. Kast, and C. J. Melief. 1996. Immunogenicity of peptides bound to MHC class I molecules depends on the MHC-peptide complex stability. J.Immunol. 156:3308.

76. Serwold, T., S. Gaw, and N. Shastri. 2001. ER aminopeptidases generate a unique pool of peptides for MHC class I molecules. Nat.Immunol. 2:644.

77. Beekman, N. J., P. A. van Veelen, T. van Hall, A. Neisig, A. Sijts, M. Camps, P. M. Kloetzel, J. J. Neefjes, C. J. Melief, and F. Ossendorp. 2000. Abrogation of CTL epitope processing by single amino acid substitution flanking the C-terminal proteasome cleavage site. J.Immunol. 164:1898.

78. Craiu, A., T. Akopian, A. Goldberg, and K. L. Rock. 1997. Two distinct proteolytic processes in the generation of a major histocompatibility complex class I-presented peptide. Proc.Natl.Acad.Sci.U.S.A 94:10850.

79. Mo, X. Y., P. Cascio, K. Lemerise, A. L. Goldberg, and K. Rock. 1999. Distinct proteolytic processes generate the C and N termini of MHC class I-binding peptides. J.Immunol. 163:5851.

80. Snyder, H. L., I. Bacik, J. W. Yewdell, T. W. Behrens, and J. R. Bennink. 1998. Promiscuous liberation of MHC-class I-binding peptides from the C termini of membrane and soluble proteins in the secretory pathway. Eur.J.Immunol. 28:1339

81. Basu, D., C. B. Williams, and P. M. Allen. 1998. In vivo antagonism of a T cell response by an endogenously expressed ligand. Proc.Natl.Acad.Sci.U.S.A 95:14332.

82. Williams, C. B., D. L. Engle, G. J. Kersh, W. J. Michael, and P. M. Allen. 1999. A kinetic threshold between negative and positive selection based on the longevity of the T cell receptor-ligand complex. J.Exp.Med. 189:1531.

83. Sant Angelo, D. B. and C. A. Janeway, Jr. 2002. Negative selection of thymocytes expressing the D10 TCR. Proc.Natl.Acad.Sci.U.S.A 99:6931.

84. Mutis, T., K. Ghoreschi, E. Schrama, J. A. Kamp, M. Heemskerk, J. H. F. Falkenburg, M. Wilke, and E. Goulmy. 2002. Efficient induction of minor histocompatibility antigen HA-1 specific cytotoxic T cells using dendritic cells retrovirally transduced with HA-1 coding cDNA. Biol. Blood Marrow Transplant. In press.

What is claimed is:

1. A peptide characterized in being immunogenic and obtainable from the minor Histocompatibility antigen HA-1, said peptide further characterized by comprising a sequence selected from the group of sequences consisting of VLXDDLLEA (SEQ ID NO:______), KECVLXDDL (SEQ ID NO:______), combinations thereof, and a derivative of any thereof having similar functional or immunological properties, wherein X represents a histidine or an arginine residue.

2. The peptide of claim 1, wherein the sequence is VLHDDLLEA (SEQ ID NO:______).

3. The peptide of claim 1, wherein the sequence is KECVLHDDL (SEQ ID NO:______).

4. A preparation comprising the peptide of claim 1.

5. A preparation comprising the peptide of claim 2.

6. A preparation comprising the peptide of claim 3.

7. A method of inducing tolerance in a subject to transplants to prevent rejection and/or Graft versus Host disease or a method treating (auto)immune disease in a subject, said method comprising:
administering the preparation of claim 4, claim 5, or claim 6, to the subject.

8. A method for the elimination of a group of hematopoietic cells, said method comprising:
presenting the peptide of claim 1 or claim 2 in the context of HLA class 1, wherein elimination is induced directly or indirectly by specific recognition of the peptide in the context of HLA class 1.

9. An analog of the peptide of claim 1, claim 2, or claim 3 wherein said analog is an antagonist for the activity of a T cell recognizing the peptide.

10. A process for producing antibodies, T cell receptors, anti-idiotypic B-cells, T-cells, or mixtures of any thereof, said process comprising:
immunizing a mammal with the peptide of claim 1, claim 2, or claim 3, and
harvesting antibodies, T cell receptors, anti-idiotypic B-cells, T-cells, or mixtures of any thereof from the mammal.

11. Antibodies, T-cell receptors, B-cells, T-cells, and or mixtures of any thereof obtainable by the process of claim 10.

12. A process for generating a cytotoxic T-cell against aminor antigen, said method comprising:
contacting a cell selected from the group of a hematopoietic cell and a dendritic cell with the peptide of claim 1, claim 2, or claim 3, thus
generating a cytotoxic T-cell against the minor antigen.

13. The process of claim 12, wherein the cell is contacted with the peptide in the context of HLA-B60.

14. The process of claim 12 or claim 13, wherein the cell is a dendritic cell.

15. The process of claim 12, claim 13, or claim 14 wherein the cell is a hematopoietic cell negative for said minor antigen.

16. The process of claim 12, claim 13, claim 14, or claim 15, wherein said minor antigen is HA-1.

17. The process of any one of claims 12-16 wherein the contacting is carried out ex vivo.

18. The process of any one of claims 12-17, wherein said cytotoxic T-cell includes a suicide gene.

19. The process of any one of claims 12-18, wherein said cytotoxic T-cell is immortalized.

20. A cytotoxic T-cell obtainable by the process of any one of claims 12-19.

21. The cytotoxic T-cell of claim 18, wherein said cytotoxic T-cell is capable of expansion.

22. A method for eliminating a non-hematopoietic tumor cell presenting an HA-1 minor histocompatibility antigen (mHag) in a context of HLA class I, said method comprising:
directly or indirectly inducing elimination by specific recognition of mHag in the context of HLA class I.

23. A method for killing a non-hematopoietic human tumor cell functionally expressing an HA-1 mHag in the context of HLA class I, said method comprising:
incubating the non-hematopoietic human tumor cell with a cytotoxic T lymphocyte specific for the HA-1 mHag presented in the context of HLA class I.

24. A method for determining whether a cell expresses functional levels of an HA-1 mHag in the context of HLA class I, comprising:
incubating said cell with a cytotoxic T lymphocyte (CTL) specific for said HA-1 mHag presented in the context of HLA class I, and
determining whether the cell and/or CTL is affected.

25. A method for marking a non-hematopoietic tumor cell, said method comprising:
incubating said cell with a molecule capable of specifically binding to an HA-1 mHag presented in the context of HLA class I, or capable of specifically binding to a nucleic acid encoding the HA-1 mHag presented in the context of HLA class I, and
marking non-hematopoietic tumor cells.

26. A non-hematopoietic tumor cell comprising:
a molecule capable of specifically binding to an HA-1 mHag presented in the context of HLA class I, or capable of specifically binding to a nucleic acid encoding said HA-1 mHag presented in the context of HLA class I.

27. A method for at least in part inhibiting expansion of a tumor cell in an individual, said tumor cell comprising a non-hematopoietic tumor cell presenting HA-1 mHag in the context of HLA class I, said method comprising:
providing the individual and the tumor cell with a cytotoxic T lymphocyte (CTL) specific for an HA-1 mHag presented in the context of HLA class I,
thus, at least in part, inhibiting expansion of the tumor cell.

28. The method according to claim 27, wherein the individual is provided with said CTL by a graft comprising hematopoietic cells from a donor.

29. The method according to claim 27, wherein the individual is provided with said CTL as a result of the induction of a Graft versus Tumor reaction in the individual.

30. The method according to any one of claims 27-29, wherein the individual is vaccinated with a preparation comprising an immunogenic amount of an HA-1 antigen.

31. A process for generating a cytotoxic T lymphocyte (CTL) capable of binding to an HA-1 mHag presented in the context of HLA class I, said process comprising:
administering to an individual having a mismatch for the HA-1 mHag presented in the context of HLA class I, a non-hematopoietic tumor cell expressing the HA-1 mHag presented in context of HLA class I,
thus generating a CTL capable of binding to an HA-1 mHag presented in the context of HLA class I.

32. A cytotoxic T lymphocyte (CTL) capable of binding to an HA-1 mHag presented in the context of HLA class I produced by the process of claim 31.

33. A method for treating a disease in a subject related at least in part to non-hematopoietic tumor cells, said method comprising:
administering to the subject an antigen specific T cell having a specificity for HA-1 presented in the context of MHC class-I or a molecule capable of specifically binding an HA-1 mHag in the context of HLA class I,
thus treating said disease.

34. A method for treating cancer in a subject caused by non-hematopoietic tumor cells, said method comprising:
administering a composition comprising an HA-1 antigen to the subject.

35. A method for inducing and/or enhancing the generation of HA-1 specific cytotoxic lymphocytes in an HA-1 negative donor of lymphocytes, said method comprising:
administering an HA-1 antigen to the HA-1 negative donor thus generating HA-1 specific cytotoxic lymphocytes.

36. The method of claim 36 wherein the HA-1 antigen comprises the peptide of claim 1 or claim 3.

37. A method for treating a disease that is at least in part related to tumor cells, said method comprising:
administering the peptide of claim 1 or claim 3 to a subject.

38. A method for the elimination of a cell selected from the group of cells consisting of hematopoietic cells, tumor cells, and hematopoietic cells and tumor cells, the cell presenting the peptide of claim 1 or claim 3 in the context of HLA-B60, comprising inducing elimination directly or indirectly by specific recognition of the peptide in the context of HLA-B60.

39. A method for killing a cell selected from the group of cells consisting of hematopoietic cells, tumor cells, and hematopoietic cells and tumor cells, the cell expressing an HA-1 mHag comprising the peptide of claim 1 or claim 3 in the context of HLA-B60, said method comprising:
incubating the cells with a cytotoxic T lymphocyte (CTL) specific for the HA-1 mHag presented in the context of HLA-B60.

40. A method for marking a cell selected from the group of cells consisting of hematopoietic cells, tumor cell, and mixtures of hematopoietic cells, said method comprising:
incubating the cell with a molecule capable of specifically binding to an HA-1 mHag comprising the peptide of claim 1 or claim 3 presented in the context of HLA-B60, or capable of specifically binding to a nucleic acid encoding said HA-1 mHag.

41. The method according to any one of claims 39-41, wherein the cell is a non15 hematopoietic tumor cell.

42. A method for determining whether a cell expresses functional levels of an HA-1 mHag comprising the peptide of claim 1 or claim 3 in the context of HLA-B60, said method comprising:
incubating the cell with a cytotoxic T lymphocyte (CTL) specific for said HA-1 mHag presented in the context of HLA-B60 and determining whether the cell and/or CTL is affected.

43. A method for at least in part inhibiting expansion of a tumor cell, said method comprising:
providing the tumor cell with a cytotoxic T lymphocyte (CTL) specific for an HA-1 mHag comprising the peptide of claim 1 or claim 3 presented in the context of HLA-B60.

44. The method according to claim 44 wherein the tumor cell is non-hematopoietic.

45. A method for generating a cytotoxic T lymphocyte (CTL) capable of binding to an HA-1 mHag comprising the peptide of claim 1 or claim 3, presented in the context of HLA-B60, said method comprising:
administering to an individual having a mismatch for the HA-1 mHag, a tumor cell expressing the HA-1 mHag presented in the context of HLA-B60.

46. The method according to claim 46, wherein the tumor cell is non-hematopoietic.

47. A method for treating a disease that is at least in part related to a tumor cell, said method comprising:
administering a molecule capable of specifically binding an HA-1 mHag comprising the peptide of claim 1 or claim 3 in the context of HLA-B60 to a subject.

48. The method according to claim 48, wherein the tumor cell is non-hematopoietic.


SEQUENCE LISTING (77 sequences)

SEQ ID NO: 1 - protein, 9 aa, Artificial Sequence
  Description of Artificial Sequence HA-1 peptide wherein X can be R or H
VLXDDLLEA

SEQ ID NO: 2 - protein, 9 aa, Artificial Sequence
  Description of Artificial Sequence HA-1 peptide
VLHDDLLEA

SEQ ID NO: 3 - protein, 9 aa, Artificial Sequence
  Description of Artificial Sequence HA-1 peptide wherein X can be R or H
KECVLXDDL

SEQ ID NO: 4 - protein, 9 aa, Artificial Sequence
  Description of Artificial Sequence HA-1 peptide
KECVLHDDL

SEQ ID NO: 5 - protein, 9 aa, Artificial Sequence
  Description of Artificial Sequence HA-1 peptide
KECVLRDDL

SEQ ID NO: 6 - protein, 10 aa, Artificial Sequence
  Description of Artificial Sequence HA-1 peptide
KECVLHDDLL

SEQ ID NO: 7 - protein, 10 aa, Artificial Sequence
  Description of Artificial Sequence HA-1 peptide
KECVLRDDLL

SEQ ID NO: 8 - protein, 10 aa, Artificial Sequence
  Description of Artificial Sequence HA-1 peptide wherein X can be R or H
KECVLXDDLL

SEQ ID NO: 9 - protein, 10 aa, Artificial Sequence
  Description of Artificial Sequence peptide capable of binding HLA-A2.1
FLPSDCFPSV

SEQ ID NO: 10 - protein, 9 aa, Artificial Sequence
  Description of Artificial Sequence HA-1 peptide
VLRDDLLEA

SEQ ID NO: 11 - protein, 9 aa, Artificial Sequence
  Description of Artificial Sequence HLA-A3 binder positive control peptide
KQSSKALQR

SEQ ID NO: 12 - protein, 9 aa, Artificial Sequence
  Description of Artificial Sequence HLA-B60 binder positive control peptide
KESTLHLVL

SEQ ID NO: 13 - protein, 9 aa, Artificial Sequence
  Description of Artificial Sequence sequence revealed from peptide candidate m/z 550 wherein X can be I or L
YXTDRVMTV

SEQ ID NO: 14 - protein, 9 aa, Artificial Sequence
  Description of Artificial Sequence sequence of peptide candidate m/z 513 wherein X can be L or I
VXHDDXXEA

SEQ ID NO: 15 - protein, 8 aa, Artificial Sequence
  Description of Artificial Sequence HA-1 peptide
VLHDLLEA

SEQ ID NO: 16 - DNA, 25 bp, Artificial Sequence
  Description of Artificial Sequence reverse primer
gctcctgcatgacgctctgtctgca

SEQ ID NO: 17 - DNA, 24 bp, Artificial Sequence
  Description of Artificial Sequence HA-1 (I) forward primer
gacgtcgtcgaggacatctcccat

SEQ ID NO: 18 - DNA, 25 bp, Artificial Sequence
  Description of Artificial Sequence reverse primer
gaaggccacagcaatcgtctccagg

SEQ ID NO: 19 - DNA, 30 bp, Artificial Sequence
  Description of Artificial Sequence primer H1
ccttgagaaacttaaggagtgtgtgctgca

SEQ ID NO: 20 - DNA, 30 bp, Artificial Sequence
  Description of Artificial Sequence primer R1
ccttgagaaacttaaggagtgtgtgttgcg

SEQ ID NO: 21 - DNA, 33 bp, Artificial Sequence
  Description of Artificial Sequence forward primer
ccggcatggacgtcgtcgaggacatctcccatc

SEQ ID NO: 22 - DNA, 30 bp, Artificial Sequence
  Description of Artificial Sequence reverse primer
ctacttcaggccacagcaatcgtctccagg

SEQ ID NO: 23 - protein, 9 aa, Artificial Sequence
  Description of Artificial Sequence HA-1 peptide
ECVLRDDLL

SEQ ID NO: 24 - protein, 10 aa, Artificial Sequence
  Description of Artificial Sequence HA-1 peptide wherein X can be H or R
VLXDDLLEAR

SEQ ID NO: 25 - DNA, 32 bp, Artificial Sequence
  Description of Artificial Sequence CFL5c8 primer wherein n is unknown
cccccccccccccccgtctagannnnnnnnnn

SEQ ID NO: 26 - DNA, 39 bp, Artificial Sequence
  Description of Artificial Sequence CFL5cT primer wherein n is unknown
cccccccccccccccgtctagatttttttttttttttvn

SEQ ID NO: 27 - DNA, 27 bp, Artificial Sequence
  Description of Artificial Sequence CP2 primer
tcagaattcatgccccccccccccccc

SEQ ID NO: 28 - DNA, 24 bp, Artificial Sequence
  Description of Artificial Sequence HA-1 (I) reverse primer
gaaggccacagcaatcgtctccag

SEQ ID NO: 29 - DNA, 21 bp, Artificial Sequence
  Description of Artificial Sequence HA-1 (II) forward primer
acactgctgtcgtgtgaagtc

SEQ ID NO: 30 - DNA, 20 bp, Artificial Sequence
  Description of Artificial Sequence HA-1 (II) reverse primer
tcaggccctgctgtactgca

SEQ ID NO: 31 - DNA, 20 bp, Artificial Sequence
  Description of Artificial Sequence CD45 forward primer
ctgaaggagaccattggtga

SEQ ID NO: 32 - DNA, 20 bp, Artificial Sequence
  Description of Artificial Sequence CD45 reverse primer
ggtactggtacacagttcga

SEQ ID NO: 33 - protein, 17 aa, Artificial Sequence
  Description of Artificial Sequence HA-1 polymorphic region wherein X can be H or R
EKLKECVLXDDLLEARR

SEQ ID NO: 34 - protein, 9 aa, Artificial Sequence
  Description of Artificial Sequence HA-1 peptide
EKLKECVLH

SEQ ID NO: 35 - protein, 9 aa, Artificial Sequence
  Description of Artificial Sequence HA-1 peptide
EKLKECVLR

SEQ ID NO: 36 - protein, 9 aa, Artificial Sequence
  Description of Artificial Sequence HA-1 peptide
KLKECVLHD

SEQ ID NO: 37 - protein, 9 aa, Artificial Sequence
  Description of Artificial Sequence HA-1 peptide
KLKECVLRD

SEQ ID NO: 38 - protein, 9 aa, Artificial Sequence
  Description of Artificial Sequence HA-1 peptide
LKECVLHDD

SEQ ID NO: 39 - protein, 9 aa, Artificial Sequence
  Description of Artificial Sequence HA-1 peptide
LKECVLRDD

SEQ ID NO: 40 - protein, 9 aa, Artificial Sequence
  Description of Artificial Sequence HA-1 peptide
ECVLHDDLL

SEQ ID NO: 41 - protein, 9 aa, Artificial Sequence
  Description of Artificial Sequence HA-1 peptide
CVLHDDLLE

SEQ ID NO: 42 - protein, 9 aa, Artificial Sequence
  Description of Artificial Sequence HA-1 peptide
CVLRDDLLE

SEQ ID NO: 43 - protein, 10 aa, Artificial Sequence
  Description of Artificial Sequence HA-1 peptide
VLHDDLLEAR

SEQ ID NO: 44 - protein, 10 aa, Artificial Sequence
  Description of Artificial Sequence HA-1 peptide
VLRDDLLEAR

SEQ ID NO: 45 - protein, 9 aa, Artificial Sequence
  Description of Artificial Sequence HA-1 peptide
LHDDLLEAR

SEQ ID NO: 46 - protein, 9 aa, Artificial Sequence
  Description of Artificial Sequence HA-1 peptide
LRDDLLEAR

SEQ ID NO: 47 - protein, 9 aa, Artificial Sequence
  Description of Artificial Sequence HA-1 peptide
HDDLLEARR

SEQ ID NO: 48 - protein, 9 aa, Artificial Sequence
  Description of Artificial Sequence HA-1 peptide
RDDLLEARR

SEQ ID NO: 49 - protein, 29 aa, Artificial Sequence
  Description of Artificial Sequence 29 amino acid long HA-1 peptide
GLEKLKECVLHDDLLEARRPRAHECLGEA

SEQ ID NO: 50 - protein, 14 aa, Artificial Sequence
  Description of Artificial Sequence HA-1 peptide
GLEKLKECVLHDDL

SEQ ID NO: 51 - protein, 27 aa, Artificial Sequence
  Description of Artificial Sequence HA-1 peptide
GLEKLKECVLHDDLLEARRPRAHECLG

SEQ ID NO: 52 - protein, 19 aa, Artificial Sequence
  Description of Artificial Sequence HA-1 peptide
HDDLLEARRPRAHECLGEA

SEQ ID NO: 53 - protein, 22 aa, Artificial Sequence
  Description of Artificial Sequence HA-1 peptide
GLEKLKECVLHDDLLEARRPRA

SEQ ID NO: 54 - protein, 13 aa, Artificial Sequence
  Description of Artificial Sequence HA-1 peptide
EKLKECVLHDDLL

SEQ ID NO: 55 - protein, 25 aa, Artificial Sequence
  Description of Artificial Sequence HA-1 peptide
GLEKLKECVLHDDLLEARRPRAHEC

SEQ ID NO: 56 - protein, 12 aa, Artificial Sequence
  Description of Artificial Sequence HA-1 peptide
GLEKLKECVLHD

SEQ ID NO: 57 - protein, 17 aa, Artificial Sequence
  Description of Artificial Sequence HA-1 peptide
GLEKLKECVLHDDLLEA

SEQ ID NO: 58 - protein, 10 aa, Artificial Sequence
  Description of Artificial Sequence HA-1 peptide
GLEKLKECVL

SEQ ID NO: 59 - protein, 29 aa, Artificial Sequence
  Description of Artificial Sequence 29 amino acid long HA-1 peptide
GLEKLKECVLRDDLLEARRPRAHECLGEA

SEQ ID NO: 60 - protein, 27 aa, Artificial Sequence
  Description of Artificial Sequence HA-1 peptide
GLEKLKECVLRDDLLEARRPRAHECLG

SEQ ID NO: 61 - protein, 28 aa, Artificial Sequence
  Description of Artificial Sequence HA-1 peptide
GLEKLKECVLRDDLLEARRPRAHECLGE

SEQ ID NO: 62 - protein, 14 aa, Artificial Sequence
  Description of Artificial Sequence HA-1 peptide
GLEKLKECVLRDDL

SEQ ID NO: 63 - protein, 22 aa, Artificial Sequence
  Description of Artificial Sequence HA-1 peptide
GLEKLKECVLRDDLLEARRPRA

SEQ ID NO: 64 - protein, 13 aa, Artificial Sequence
  Description of Artificial Sequence HA-1 peptide
EKLKECVLRDDLL

SEQ ID NO: 65 - protein, 12 aa, Artificial Sequence
  Description of Artificial Sequence HA-1 peptide
CVLRDDLLEARR

SEQ ID NO: 66 - protein, 26 aa, Artificial Sequence
  Description of Artificial Sequence HA-1 peptide
GLEKLKECVLRDDLLEARRPRAHECL

SEQ ID NO: 67 - protein, 25 aa, Artificial Sequence
  Description of Artificial Sequence HA-1 peptide
GLEKLKECVLRDDLLEARRPRAHEC

SEQ ID NO: 68 - protein, 12 aa, Artificial Sequence
  Description of Artificial Sequence HA-1 peptide
GLEKLKECVLRD

SEQ ID NO: 69 - protein, 17 aa, Artificial Sequence
  Description of Artificial Sequence HA-1 peptide
GLEKLKECVLRDDLLEA

SEQ ID NO: 70 - protein, 19 aa, Artificial Sequence
  Description of Artificial Sequence HA-1 peptide
GLEKLKECVLRDDLLEARR

SEQ ID NO: 71 - protein, 21 aa, Artificial Sequence
  Description of Artificial Sequence HA-1 peptide
GLEKLKECVLRDDLLEARRPR

SEQ ID NO: 72 - protein, 23 aa, Artificial Sequence
  Description of Artificial Sequence HA-1 peptide
GLEKLKECVLRDDLLEARRPRAH

SEQ ID NO: 73 - DNA, 38 bp, Artificial Sequence
  Description of Artificial Sequence KIAA0223 sequence derived from a presumed HA-1 negative individual
gagtgtgtgttgcgtgacgacctccttgaggcccgccg

SEQ ID NO: 74 - protein, 13 aa, Artificial Sequence
  Description of Artificial Sequence KIAA0223 sequence derived from a presumed HA-1 negative individual
ECVLRDDLLEARR

SEQ ID NO: 75 - DNA, 38 bp, Artificial Sequence
  Description of Artificial Sequence KIAA0223 sequence derived from a presumed HA-1 homozygous positive individual
gagtgtgtgctgcatgacgacctccttgaggcccgccg

SEQ ID NO: 76 - protein, 13 aa, Artificial Sequence
  Description of Artificial Sequence KIAA0223 sequence derived from a presumed HA-1 homozygous positive individual
ECVLHDDLLEARR

SEQ ID NO: 77 - protein, 9 aa, Artificial Sequence
  Description of Artificial Sequence HA-1 peptide
YIGEVLVSV